(19) United States
(12) Patent Application Publication
(10) Pub. No.: US 2011/0092544 A1
(43) Pub. Date: Apr. 21, 2011

(54) FUNGICIDAL PYRIDINES

(75) Inventor: Bruce Lawrence Finkelstein, Newark, DE (US)

(73) Assignee: E.I. DU PONT DE nemours and company, Wilmington, DE (US)

(21) Appl. No.: 12/993,217

(22) PCT Filed: Jun. 18, 2009
(86) PCT No.: PCT/US2009/047724

§ 371 Date: Nov. 17, 2010

Related U.S. Application Data
(60) Provisional application No. 61/076,348, filed on Jun. 27, 2008.

(51) Int. Cl. A01N 43/40; C07D 213/80; C07D 213/50; C07D 213/38; C07D 213/42; C07D 213/57; C07D 401/04; C07D 413/04; A01N 43/836; A01P 3/00

(52) U.S. Cl. 514/334; 546/322; 514/356; 514/355; 546/315; 546/339; 514/277; 546/344; 546/330; 514/357; 546/257; 546/269.4; 514/340; 546/258

(57) ABSTRACT

Disclosed are compounds of Formula 1, N-oxides, and salts thereof,

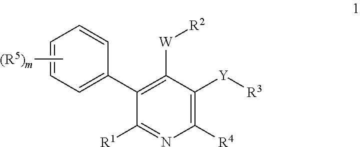

wherein
each W and Y is independently CH₂, O, C(═O), S(═O)ₙ, NR⁸ or a direct bond;
R⁴ is H, halogen, cyano, hydroxy, C₁-C₂ alkyl, C₁-C₂ haloalkyl, C₂ alkenyl, C₂ haloalkenyl or C₂ alkynyl;
m is an integer selected from 0, 1, 2, 3, 4 and 5; and
R¹, R², R³, R⁵, R⁸ and n are as defined in the disclosure.
   Also disclosed are compositions containing the compounds of Formula 1 and methods for controlling plant disease caused by a fungal pathogen comprising applying an effective amount of a compound or a composition of the invention.

### FIELD OF THE INVENTION

This invention relates to certain pyridines, their N-oxides, salts and compositions, and methods of their use as fungicides.

### BACKGROUND OF THE INVENTION

The control of plant diseases caused by fungal plant pathogens is extremely important in achieving high crop efficiency. Plant disease damage to ornamental, vegetable, field, cereal, and fruit crops can cause significant reduction in productivity and thereby result in increased costs to the consumer. Many products are commercially available for these purposes, but the need continues for new compounds which are more effective, less costly, less toxic, environmentally safer or have different sites of action.

### SUMMARY OF THE INVENTION

This invention is directed to compounds of Formula 1 (including all stereoisomers such as enantiomers, diastereomers, atropisomers and geometric isomers), N-oxides, and salts thereof, agricultural compositions containing them and their use as fungicides:

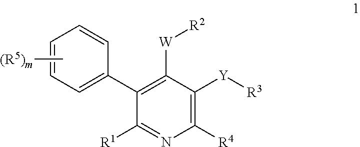

wherein

R¹ is halogen, cyano, hydroxy, amino, C₁-C₄ alkyl, C₂-C₄ alkenyl, C₂-C₄ alkynyl, C₁-C₄ haloalkyl, C₂-C₄ haloalkenyl, C₂-C₄ haloalkynyl, cyclopropyl, halocyclopropyl, C₂-C₄ alkoxyalkyl, C₂-C₄ alkylthioalkyl, C₂-C₄ alkylsulfinylalkyl, C₂-C₄ alkylsulfonylalkyl, C₂-C₄ alkylcarbonyl, C₂-C₄ alkoxycarbonyl, C₁-C₃ hydroxyalkyl, C₁-C₃ alkoxy, C₁-C₃ haloalkoxy, C₁-C₃ alkylthio, C₁-C₃ haloalkylthio, C₁-C₃ alkylsulfinyl, C₁-C₃ haloalkylsulfinyl, C₁-C₃ alkylsulfonyl, C₁-C₃ haloalkylsulfonyl, C₁-C₃ alkylamino or C₂-C₄ dialkylamino;
each W and Y is independently CH₂, O, C(═O), S(═O)ₙ, NR⁸ or a direct bond;
R² is a phenyl ring optionally substituted with up to 5 substituents independently selected from R⁶; or a 3-, 4-, 5- or 6-membered heterocyclic ring containing ring members selected from carbon atoms and up to 4 heteroatoms selected from up to 2 oxygen, up to 2 sulfur and up to 3 nitrogen atoms, wherein up to 3 carbon atom ring members are independently selected from C(═O) and C(═S), and the sulfur atom ring members are independently selected from S(═O)ₚ(═NR⁹)_{q}, the heterocyclic ring optionally substituted with up to 5 substituents independently selected from R⁶ on carbon atom ring members and R^{6a} on nitrogen atom ring members;
R³ is a phenyl ring optionally substituted with up to 5 substituents independently selected from R⁷; or a 3-, 4-, 5- or 6-membered heterocyclic ring containing ring members selected from carbon atoms and up to 4 heteroatoms selected from up to 2 oxygen, up to 2 sulfur and up to 3 nitrogen atoms, wherein up to 3 carbon atom ring members are independently selected from C(═O) and C(═S), and the sulfur atom ring members are independently selected from S(═O)ₚ(═NR⁹)_{q}, the heterocyclic ring optionally substituted with up to 5 substituents independently selected from R⁷ on carbon atom ring members and R^{7a} on nitrogen atom ring members; or
when Y is a direct bond, then R³ is also selected from halogen, cyano, hydroxy, amino, nitro, —CHO, C₁-C₆ alkyl, C₂-C₆ alkenyl, C₂-C₆ alkynyl, C₁-C₆ haloalkyl, C₂-C₆ haloalkenyl, C₂-C₆ haloalkynyl, C₃-C₆ cycloalkyl, C₃-C₆ halocycloalkyl, C₄-C₈ alkylcycloalkyl, C₄-C₈ cycloalkylalkyl, C₆-C₁₂ cycloalkylcycloalkyl, C₄-C₈ halocycloalkylalkyl, C₅-C₈ alkylcycloalkylalkyl, C₃-C₆ cycloalkenyl, C₂-C₆ alkoxyalkyl, C₂-C₆ alkylthioalkyl, C₂-C₆ alkylsulfinylalkyl, C₂-C₆ alkylsulfonylalkyl, C₂-C₆ alkylaminoalkyl, C₃-C₆ dialkylaminoalkyl, C₂-C₆ alkylcarbonyl, C₂-C₆ haloalkylcarbonyl, C₄-C₆ cycloalkylcarbonyl, C₂-C₆ alkoxycarbonyl, C₂-C₆ alkylaminocarbonyl, C₃-C₈ dialkylaminocarbonyl, C₂-C₆ cyanoalkyl, C₁-C₆ hydroxyalkyl, C₂-C₆ hydroxyhaloalkyl, C₂-C₆ hydroxyalkylcarbonyl, C₂-C₆ hydroxycarbonylalkyl, C₁-C₆ alkoxy, C₁-C₆ haloalkoxy, C₃-C₆ cycloalkoxy, C₃-C₆ halocycloalkoxy, C₂-C₆ alkoxyalkoxy, C₃-C₆ alkoxycarbonylalkyl, C₁-C₆ alkylthio, C₁-C₆ haloalkylthio, C₁-C₆ alkylsulfinyl, C₁-C₆ haloalkylsulfinyl, C₁-C₆ alkylsulfonyl, C₁-C₆ haloalkylsulfonyl, C₃-C₉ trialkylsilyl, C₁-C₆ alkylamino, C₂-C₆ dialkylamino, C₂-C₆ haloalkylamino, C₂-C₆ halodialkylamino, C₃-C₆ cycloalkylamino, C₂-C₆ alkylcarbonylamino, C₂-C₆ haloalkylcarbonylamino, C₁-C₆ alkylsulfonylamino and C₁-C₆ haloalkylsulfonylamino;
R⁴ is H, halogen, cyano, hydroxy, C₁-C₂ alkyl, C₁-C₂ haloalkyl, C₂ alkenyl, C₂ haloalkenyl or C₂ alkynyl;
each R⁵, R⁶ and R⁷ is independently halogen, cyano, hydroxy, amino, nitro, —CHO, C₁-C₆ alkyl, C₂-C₆ alkenyl, C₂-C₆ alkynyl, C₁-C₆ haloalkyl, C₂-C₆ alkylcarbonyl, C₂-C₆ haloalkylcarbonyl, C₂-C₆ alkoxycarbonyl, C₂-C₆ alkylaminocarbonyl, C₃-C₆ dialkylaminocarbonyl, C₂-C₆ alkylaminoalkoxy, C₂-C₆ haloalkenyl, C₂-C₆ haloalkynyl, C₃-C₆ cycloalkyl, C₃-C₆ halocycloalkyl, C₄-C₈ alkylcycloalkyl, C₄-C₈ cycloalkylalkyl, C₅-C₈ alkylcycloalkylalkyl, C₂-C₆ alkoxyalkyl, C₂-C₆ cyanoalkyl, C₁-C₆ hydroxyalkyl, C₁-C₆ alkoxy, C₁-C₆ haloalkoxy, C₃-C₆ cycloalkoxy, C₃-C₆ halocycloalkoxy, C₂-C₆ alkylcarbonyloxy, C₁-C₆ alkylthio, C₁-C₆ haloalkylthio, C₁-C₆ alkylsulfinyl, C₁-C₆ haloalkylsulfinyl, C₁-C₆ alkylsulfonyl, C₁-C₆ haloalkylsulfonyl, C₃-C₉ trialkylsilyl, C₂-C₆ alkylcarbonylthio, C₁-C₆ alkylamino or C₂-C₆ dialkylamino;
each R^{6a} and R^{7a} is independently cyano, C₁-C₆ alkyl, C₂-C₆ alkenyl, C₂-C₆ alkynyl, C₁-C₆ haloalkyl, C₂-C₆ alkylcarbonyl, C₂-C₆ haloalkylcarbonyl, C₂-C₆ alkoxycarbonyl, C₂-C₆ alkylaminocarbonyl, C₃-C₆ dialkylaminocarbonyl, C₂-C₆ haloalkenyl, C₂-C₆ haloalkynyl, C₃-C₆ cycloalkyl, C₃-C₆ halocycloalkyl, C₄-C₈ alkylcycloalkyl, C₄-C₈ cycloalkylalkyl, C₅-C₈ alkylcycloalkylalkyl, C₂-C₆ alkoxyalkyl, C₁-C₆ alkoxy, C₁-C₆ haloalkoxy, C₃-C₆ cycloalkoxy, C₃-C₆ halocycloalkoxy, C₁-C₆ alkylthio, C₁-C₆ haloalkylthio, C₁-C₆ alkylsulfonyl, C₁-C₆ haloalkylsulfonyl or C₃-C₉ trialkylsilyl; or
one pair of R⁵ substituents attached to adjacent ring atoms, one pair of substituents selected from R⁶ and R^{6a} substituents attached to adjacent ring atoms, and one pair of substituents selected from R⁷ and R^{7a} substituents attached to adjacent ring atoms may each be independently taken together with the atoms to which they are attached to form a 5-, 6- or 7-membered fused ring, each fused ring containing ring members selected from carbon and up to 4 heteroatoms selected from up to 2 oxygen, up to 2 sulfur and up to 3 nitrogen, and optionally substituted with up to 3 substituents independently selected from the group consisting of C₁-C₂ alkyl, halogen, cyano, nitro and C₁-C₂ alkoxy on carbon ring members and from the group consisting of C₁-C₂ alkyl, cyano and C₁-C₂ alkoxy on nitrogen ring members; or
one pair of R⁶ substituents attached to the same ring atom and one pair of R⁷ substituents attached to the same ring atom may each be independently taken together with the atom to which they are attached to form a 5-, 6- or 7-membered spirocyclic ring, each spirocyclic ring containing ring members selected from carbon, up to 4 heteroatoms selected from up to 2 oxygen, up to 2 sulfur and up to 3 nitrogen, and optionally substituted with up to 3 substituents independently selected from the group consisting of C₁-C₂ alkyl, halogen, cyano, nitro and C₁-C₂ alkoxy on carbon ring members and from the group consisting of C₁-C₂ alkyl, cyano and C₁-C₂ alkoxy on nitrogen ring members;
each R⁸ and R⁹ is independently H or C₁-C₃ alkyl;
m is an integer selected from 0, 1, 2, 3, 4 and 5;
each n is independently an integer selected from 0, 1 and 2; and
p and q are independently 0, 1 or 2 in each instance of S(═O)ₚ(═NR⁹)_{q}, provided that the sum of p and q is 0, 1 or 2;
provided that when Y is a direct bond and R³ is a phenyl ring substituted with two alkoxy substituents attached at the meta positions, then R⁴ is H. More particularly, this invention pertains to a compound of Formula 1 (including all stereoisomers), an N-oxide or a salt thereof.

This invention also relates to a fungicidal composition comprising a compound of Formula 1 (i.e. in a fungicidally effective amount) and at least one additional component selected from the group consisting of surfactants, solid diluents and liquid diluents.

This invention also relates to a fungicidal composition comprising a mixture of a compound of Formula 1 and at least one other fungicide (e.g., at least one other fungicide having a different site of action).

This invention further relates to a method for controlling plant diseases caused by fungal plant pathogens comprising applying to the plant or portion thereof, or to the plant seed, a fungicidally effective amount of a compound of the invention (e.g., as a composition described herein).

### DETAILS OF THE INVENTION

As used herein, the terms “comprises,” “comprising,” “includes,” “including,” “has,” “having,” “contains”, “containing,” “characterized by” or any other variation thereof, are intended to cover a non-exclusive inclusion, subject to any limitation explicitly indicated. For example, a composition, process, method, article, or apparatus that comprises a list of elements is not necessarily limited to only those elements but may include other elements not expressly listed or inherent to such composition, process, method, article, or apparatus.

The transitional phrase “consisting of” excludes any element, step, or ingredient not specified. If in the claim, such would close the claim to the inclusion of materials other than those recited except for impurities ordinarily associated therewith. When the phrase “consists of” appears in a clause of the body of a claim, rather than immediately following the preamble, it limits only the element set forth in that clause; other elements are not excluded from the claim as a whole.

The transitional phrase “consisting essentially of” is used to define a composition or method that includes materials, steps, features, components, or elements, in addition to those literally disclosed, provided that these additional materials, steps, features, components, or elements do materially affect the basic and novel characteristic(s) of the claimed invention. The term “consisting essentially of” occupies a middle ground between “comprising” and “consisting of”.

Where applicants have defined an invention or a portion thereof with an open-ended term such as “comprising,” it should be readily understood that (unless otherwise stated) the description should be interpreted to also describe such an invention using the terms “consisting essentially of” or “consisting of.”

Further, unless expressly stated to the contrary, “or” refers to an inclusive or and not to an exclusive or. For example, a condition A or B is satisfied by any one of the following: A is true (or present) and B is false (or not present), A is false (or not present) and B is true (or present), and both A and B are true (or present).

Also, the indefinite articles “a” and “an” preceding an element or component of the invention are intended to be nonrestrictive regarding the number of instances (i.e. occurrences) of the element or component. Therefore “a” or “an” should be read to include one or at least one, and the singular word form of the element or component also includes the plural unless the number is obviously meant to be singular.

As referred to in the present disclosure and claims, “plant” includes members of Kingdom Plantae, particularly seed plants (Spermatopsida), at all life stages, including young plants (e.g., germinating seeds developing into seedlings) and mature, reproductive stages (e.g., plants producing flowers and seeds). Portions of plants include geotropic members typically growing beneath the surface of the growing medium (e.g., soil), such as roots, tubers, bulbs and corms, and also members growing above the growing medium, such as foliage (including stems and leaves), flowers, fruits and seeds.

As referred to herein, the term “seedling”, used either alone or in a combination of words means a young plant developing from the embryo of a seed.

In the above recitations, the term “alkyl”, used either alone or in compound words such as “alkylthio” or “haloalkyl” includes straight-chain or branched alkyl, such as, methyl, ethyl, n-propyl, i-propyl, or the different butyl, pentyl or hexyl isomers. “Alkenyl” includes straight-chain or branched alkenes such as ethenyl, 1-propenyl, 2-propenyl, and the different butenyl, pentenyl and hexenyl isomers. “Alkenyl” also includes polyenes such as 1,2-propadienyl and 2,4-hexadienyl. “Alkynyl” includes straight-chain or branched alkynes such as ethynyl, 1-propynyl, 2-propynyl and the different butynyl, pentynyl and hexynyl isomers. “Alkynyl” can also include moieties comprised of multiple triple bonds such as 2,5-hexadiynyl.

“Alkoxy” includes, for example, methoxy, ethoxy, n-propyloxy, isopropyloxy and the different butoxy, pentoxy and hexyloxy isomers. “Alkoxyalkyl” denotes alkoxy substitution on alkyl. Examples of “alkoxyalkyl” include CH₃OCH₂—, CH₃OCH₂CH₂—, CH₃CH₂OCH₂—, CH₃CH₂CH₂CH₂OCH₂— and CH₃OCH₂(CH₃)CHCH₂—. “Alkoxyalkoxy” denotes at least one straight-chain or branched alkoxy substitution on a straight-chain or branched alkoxy. Examples of “alkoxyalkoxy” include CH₃OCH₂O—, CH₃OCH₂(CH₃O)CHCH₂O— and (CH₃)₂CHOCH₂CH₂O—. “Alkylthio” includes branched or straight-chain alkylthio moieties such as methylthio, ethylthio, and the different propylthio, butylthio, pentylthio and hexylthio isomers. “Alkylsulfinyl” includes both enantiomers of an alkylsulfinyl group. Examples of “alkylsulfinyl” include CH₃S(O)—, CH₃CH₂S(O)—, CH₃CH₂CH₂S(O)—, (CH₃)₂CHS(O)— and the different butylsulfinyl, pentylsulfinyl and hexylsulfinyl isomers. Examples of “alkylsulfonyl” include CH₃S(O)₂—, CH₃CH₂S(O)₂—, CH₃CH₂CH₂S(O)₂—, (CH₃)₂CHS(O)₂—, and the different butylsulfonyl, pentylsulfonyl and hexylsulfonyl isomers.

“Alkylthioalkyl” denotes alkylthio substitution on alkyl. Examples of “alkylthioalkyl” include CH₃SCH₂—, CH₃SCH₂CH₂—, CH₃CH₂SCH₂—, CH₃CH₂CH₂CH₂SCH₂— and CH₃CH₂SCH₂CH₂— and other alkyl moieties bonded to sulfur, such as straight-chain or branched alkyl groups; “alkylsulfinylalkyl” and “alkylsulfonylalkyl” include the corresponding sulfoxides and sulfones, respectively. “Alkylaminoalkyl” denotes alkylamino substitution on an alkyl moiety. Examples of “alkylaminoalkyl” include propylaminomethyl, butylaminoethyl, and other alkyl moieties bonded to nitrogen, such as straight-chain or branched alkyl groups. The term “dialkylaminoalkyl” is defined analogously to the term “alkylaminoalkyl”.

“Cyanoalkyl” denotes an alkyl group substituted with one cyano group. Examples of “cyanoalkyl” include NCCH₂—, NCCH₂CH₂— and CH₃CH(CN)CH₂—. “Hydroxyalkyl” denotes an alkyl group substituted with one hydroxy group. Examples of “hydroxyalkyl” include HOCH₂CH₂—, CH₃CH₂(OH)CH— and HOCH₂CH₂CH₂CH₂—.

“Cycloalkyl” includes, for example, cyclopropyl, cyclobutyl, cyclopentyl and cyclohexyl. The term “alkylcycloalkyl” denotes alkyl substitution on a cycloalkyl moiety and includes, for example, ethylcyclopropyl, i-propylcyclobutyl, 3-methylcyclopentyl and 4-methylcyclohexyl. The term “cycloalkylalkyl” denotes cycloalkyl substitution on an alkyl group. Examples of “cycloalkylalkyl” include cyclopropylmethyl, cyclopentylethyl, and other cycloalkyl moieties bonded to straight-chain or branched alkyl groups. “Alkylcycloalkylalkyl” denotes alkyl substitution on a cycloalkylalkyl moiety. Examples include 4-methylcyclohexylmethyl and 3-ethylcyclopentylmethyl. The term “cycloalkoxy” denotes cycloalkyl linked through an oxygen atom such as cyclopentyloxy and cyclohexyloxy. “Cycloalkenyl” includes groups such as cyclopentenyl and cyclohexenyl as well as groups with more than one double bond such as 1,3- and 1,4-cyclohexadienyl.

The term “cycloalkylcycloalkyl” denotes cycloalkyl substitution on another cycloalkyl ring, wherein each cycloalkyl ring independently has from 3 to 6 carbon ring members. Examples of cycloalkylcycloalkyl radicals include cyclopropylcyclopropyl (such as 1,1′-bicyclopropyl-1-yl, 1,1′-bicyclopropyl-2-yl), cyclohexylcyclopentyl (such as 4-cyclopentylcyclohexyl) and cyclohexylcyclohexyl (such as 1,1′-bicyclohexyl-1-yl), and the different cis- or trans-cycloalkylcycloalkyl isomers, (such as (1R,2S)-1,1′-bicyclopropyl-2-yl and (1R,2R)-1,1′-bicyclopropyl-2-yl).

The term “halogen”, either alone or in compound words such as “haloalkyl” includes fluorine, chlorine, bromine or iodine. Further, when used in compound words such as “haloalkyl” said alkyl may be partially or fully substituted with halogen atoms which may be the same or different. Examples of “haloalkyl” include F₃C—, ClCH₂—, CF₃CH₂— and CF₃CCl₂—. The terms “halocycloalkyl”, “halocycloalkylalkyl”, “haloalkoxy”, “halocycloalkoxy” “haloalkylthio”, “haloalkenyl”, “haloalkynyl”, “haloalkylsulfinyl”, “haloalkylsulfonyl”, hydroxyhaloalkyl, and the like, are defined analogously to the term “haloalkyl”. Examples of “haloalkoxy” include CF₃O—, CCl₃CH₂O—, HCF₂CH₂CH₂O— and CF₃CH₂O—. Examples of “haloalkylthio” include CCl₃S—, CF₃S—, CCl₃CH₂S— and ClCH₂CH₂CH₂S—. Examples of “haloalkenyl” include (Cl)₂C═CHCH₂— and CF₃CH₂CH═CHCH₂—. Examples of “haloalkynyl” include HC≡CCHCl—, CF₃C≡C—, CCl₃C≡C—and FCH₂C≡CCH₂—. Examples of “haloalkylsulfinyl” include CF₃S(O)—, CCl₃S(O)—, CF₃CH₂S(O)— and CF₃CF₂S(O)—. Examples of “haloalkylsulfonyl” include CF₃S(O)₂—, CCl₃S(O)₂—, CF₃CH₂S(O)₂— and CF₃CF₂S(O)₂—.

“Alkylcarbonyl” denotes straight-chain or branched alkyl groups bonded to a C(═O) moiety. Examples of “alkylcarbonyl” include CH₃C(═O)—, CH₃CH₂CH₂C(═O)— and (CH₃)₂CHC(═O)—. Examples of “haloalkylcarbonyl” include CF₃C(═O)—, CH₃CCl₂C(═O)—, CCl₃CH₂CH₂C(═O)— and CF₃CF₂C(═O)—. Examples of “alkoxycarbonyl” include CH₃C(═O)—, CH₃CH₂OC(═O)—, CH₃CH₂CH₂C(═O)—, (CH₃)₂CHOC(═O)— and the different butoxy- or pentoxycarbonyl isomers. Examples of “alkylaminocarbonyl” include CH₃NHC(═O)—, CH₃CH₂NHC(═O)—, CH₃CH₂CH₂NHC(═O)—, (CH₃)₂CHNHC(═O)— and the different butylamino- or pentylaminocarbonyl isomers. Examples of “dialkylaminocarbonyl” include (CH₃)₂NC(═O)—, (CH₃CH₂)₂NC(═O)—, CH₃CH₂(CH₃)NC(═O)—, (CH₃)₂CHN(CH₃)C(═O)— and CH₃CH₂CH₂(CH₃)NC(═O)—.

Alkoxycarbonylalkyl” denotes straight-chain or branched alkoxycarbonyl substitution on a straight-chain or branched alkyl. Examples of “alkoxycarbonylalkyl” include CH₃C(═O)CH₂CH(CH₃)—, CH₃CH₂C(═O)CH₂CH₂—, (CH₃)₂CHOC(═O)CH₂—. “Alkylcarbonylthio” denotes straight-chain or branched alkylcarbonyl attached to and linked through a sulfur atom. Examples of “alkylcarbonylthio” include CH₃C(═O)S—, CH₃CH₂CH₂C(═O)S— and (CH₃)₂CHC(═O)S—.

“Alkylamino includes an NH radical substituted with straight-chain or branched alkyl. Examples of “alkylamino” include CH₃CH₂NH—, CH₃CH₂CH₂NH—, and (CH₃)₂CHCH₂NH—. Examples of “dialkylamino” include (CH₃)₂N—, (CH₃CH₂CH₂)₂N— and CH₃CH₂(CH₃)N—. The term “haloalkylamino” denotes at least one halogen group substituted on the alkyl moiety of the alkylamino group. Examples of “haloalkylamino” include CH₂ClCH₂NH— and (CF₃)₂CHNH—. “Halodialkylamino” denotes at least one alkyl moiety of the dialkylamino group is substituted with at least one halogen atom. Examples of “halodialkylamino” include CF₃(CH₃)N—, (CF₃)₂N— and CH₂Cl(CH₃)N—. “Cycloalkylamino” means the amino nitrogen atom is attached to a cycloalkyl radical and a hydrogen atom. Examples of “cycloalkylamino” include cyclopropylamino, cyclobutylamino, cyclopentylamino and cyclohexylamino. “Alkylcarbonylamino” means the amino nitrogen atom is attached to a straight-chain or branched alkylcarbonyl group and a hydrogen atom. Examples of “alkylcarbonylamino” include CH₃C(═O)NH—, CH₃CH₂C(═O)NH—, CH₃CH₂CH₂C(═O)NH— and (CH₃)₂CHC(═O)NH—. The term “haloalkylcarbonylamino” denotes at least one halogen substituted on the alkyl moiety of the alkylcarbonylamino group. Examples of “haloalkylcarbonylamino” include CH₂ClCH₂C(═O)NH—, (CH₃)₂CClC(═O)NH— and CH₂ClC(═O)NH—. “Alkylsulfonylamino” and “haloalkylsulfonylamino” are defined analogously to the term “alkylcarbonylamino”.

The term “alkylaminoalkoxy” denotes straight-chain or branched alkylamino substitution on a straight-chain or branched alkoxy radical. Examples of “alkylaminoalkoxy” include CH₃NHCH₂CH₂CH₂O—, CH₃NHCH₂CH₂O—, CH₃CH(CH₃)NHCH₂CH₂O—, CH₃CH₂CH₂CH₂NHCH₂CH₂O— and CH₃NHCH₂CH(CH₃)CH₂O—.

“Trialkylsilyl” includes 3 branched and/or straight-chain alkyl radicals attached to and linked through a silicon atom, such as trimethylsilyl, triethylsilyl and tert-butyldimethylsilyl.

The total number of carbon atoms in a substituent group is indicated by the “Cᵢ-Cⱼ” prefix where i and j are numbers from 1 to 12. For example, C₁-C₄ alkylsulfonyl designates methylsulfonyl through butylsulfonyl; C₂ alkoxyalkyl designates CH₃OCH₂—; C₃ alkoxyalkyl designates, for example, CH₃CH(OCH₃)—, CH₃OCH₂CH₂— or CH₃CH₂OCH₂—; and C₄ alkoxyalkyl designates the various isomers of an alkyl group substituted with an alkoxy group containing a total of four carbon atoms, examples including CH₃CH₂CH₂OCH₂— and CH₃CH₂OCH₂CH₂—.

When a compound is substituted with a substituent bearing a subscript that indicates the number of said substituents can exceed 1, said substituents (when they exceed 1) are independently selected from the group of defined substituents, for example, (R⁵)ₘ wherein m is 1, 2, 3, 4 or 5. When a variable group is shown to be optionally attached to a position, for example, (R⁵)ₘ wherein m may be 0, then hydrogen may be at the position even if not recited in the variable group definition. When one or more positions on a group are said to be “not substituted” or “unsubstituted”, then hydrogen atoms are attached to take up any free valency.

Unless otherwise indicated, a “ring” or “ring system” as a component of Formula 1 (e.g., substituent R²) is carbocyclic (e.g., phenyl) or heterocyclic (e.g., pyridinyl). The term “ring system” denotes two or more fused rings.

The terms “heterocyclic ring” or “heterocycle” denote a ring or ring system in which at least one atom forming the ring backbone is not carbon, e.g., nitrogen, oxygen or sulfur. Typically a heterocyclic ring contains no more than 3 nitrogens, no more than 2 oxygens and no more than 2 sulfurs. Unless otherwise indicated, a heterocyclic ring can be a saturated, partially unsaturated, or fully unsaturated ring. When a fully unsaturated heterocyclic ring satisfies Hiickel's rule, then said ring is also called a “heteroaromatic ring” or “aromatic heterocyclic ring”. Unless otherwise indicated, heterocyclic rings and ring systems can be attached through any available carbon or nitrogen by replacement of a hydrogen on said carbon or nitrogen.

The term “ring member” refers to an atom (e.g., N or O) or other moiety (e.g., C(═O), C(═S) or S(═O)_{P} (═NR⁹)_{q}) forming the backbone of a ring or ring system.

The term “spirocyclic ring” denotes a ring connected at a single atom to another ring on Formula 1 (so the rings have a single atom in common). Illustrative of a spirocyclic rings are ring systems J-1 through J-8 depicted in Exhibit 4.

“Aromatic” indicates that each of the ring atoms is essentially in the same plane and has a p-orbital perpendicular to the ring plane, and that (4n+2) π electrons, where n is a positive integer, are associated with the ring to comply with Hückel's rule.

As used herein, the following definitions shall apply unless otherwise indicated. The term “optionally substituted” is used interchangeably with the phrase “substituted or unsubstituted” or with the term “(un)substituted.” Unless otherwise indicated, an optionally substituted group may have a substituent at each substitutable position of the group, and each substitution is independent of the other.

When R² or R³ is a 3-, 4-, 5- or 6-membered nitrogen-containing heterocyclic ring, it may be attached to the remainder of Formula 1 though any available carbon or nitrogen ring atom, unless otherwise described.

As noted above, R² and R³ can independently be (among others) phenyl optionally substituted with up to 5 substituents selected from a group of substituents as defined in the Summary of Invention. An example of phenyl optionally substituted with up to five substituents is the ring illustrated as U-1 in Exhibit 1, wherein R^{v} is selected from a group of substituents as defined in the Summary of the Invention for R² and R³ (i.e. R⁶ on an R² ring, and R⁷ on an R³ ring) and r is an integer from 0 to 5.

Also, as noted above, R² and R³ can independently be (among others) a 3-, 4-, 5- or 6-membered heterocyclic ring, which may be saturated, partially unsaturated, or fully unsaturated and optionally substituted with up to 5 substituents selected from a group of substituents as defined in the Summary of Invention for R² and R³. Optionally up to 3 carbon atom ring members of the heterocyclic ring are independently selected from C(═O), C(═S) and S(═O)_{P} (═NR⁸)_{q}. The definition of S(═O)_{P} (═NR⁸)_{q} includes the possibility of unoxidized sulfur atoms as ring members, because p and q can both be zero.

Examples of a 3-, 4-, 5- or 6-membered fully unsaturated heterocyclic ring include the rings U-2 through U-67 illustrated in Exhibit 1 wherein R^{v} is any substituent as defined in the Summary of the Invention for R² or R³ (i.e. R⁶ on carbon ring members and R^{6a} on nitrogen ring members of the R² ring, and R⁷ on carbon ring members and R^{7a} on nitrogen ring members of the R³ ring) and r is an integer from 0 to 5, limited by the number of available positions on each U-ring. As U-35, U-36, U-42, U-43, U-44, U-45, U-46, U-47, U-48 and U-49 have only one available position, for these U-rings r is limited to the integers 0 or 1, and r being 0 means that the U-ring is unsubstituted and a hydrogen is present at the position indicated by (R^{v})ᵣ.

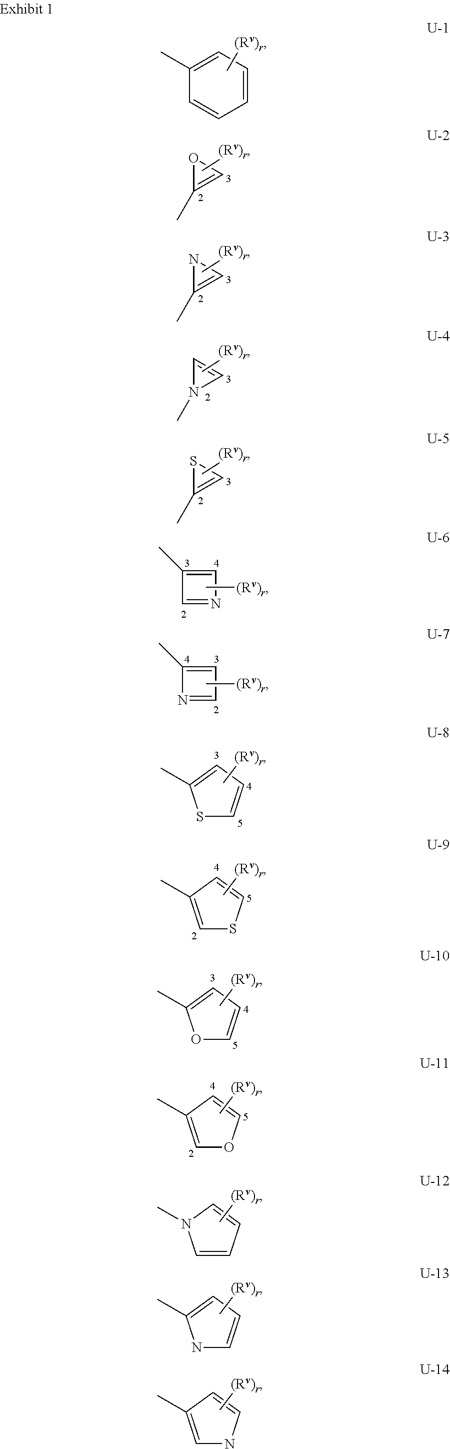

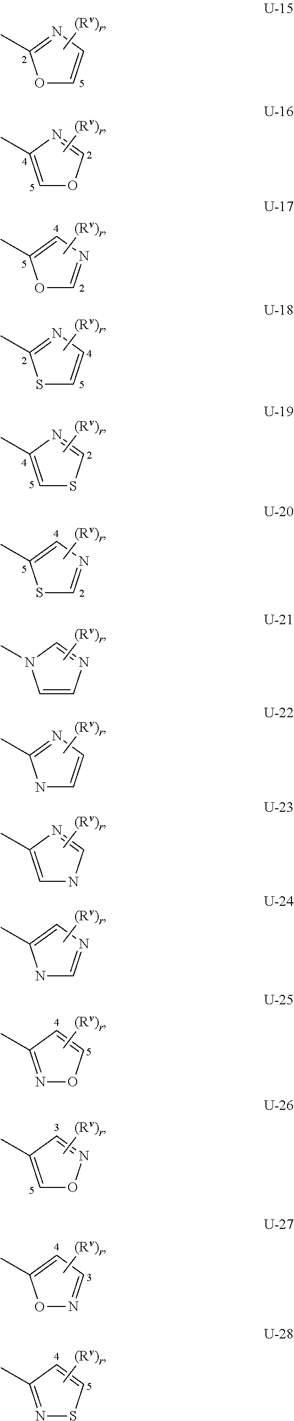

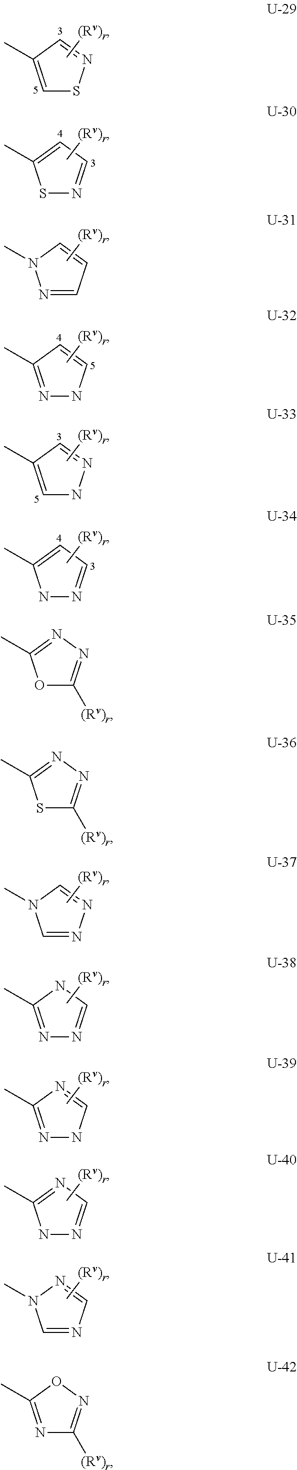

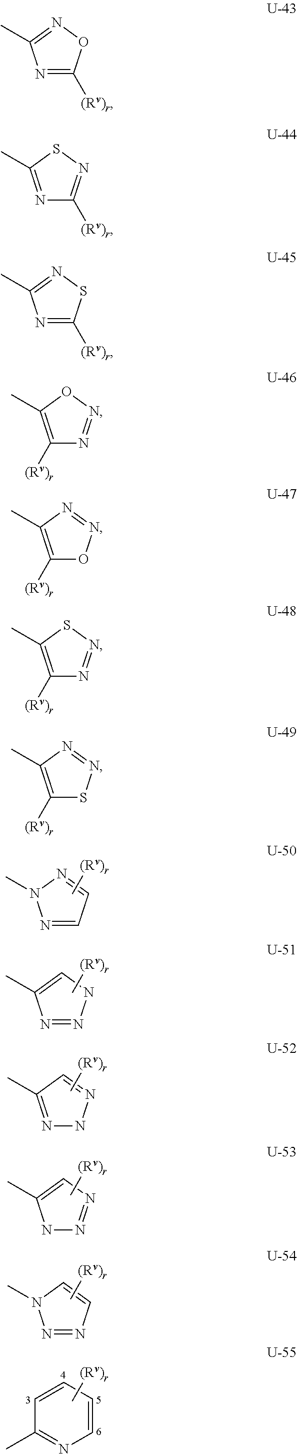

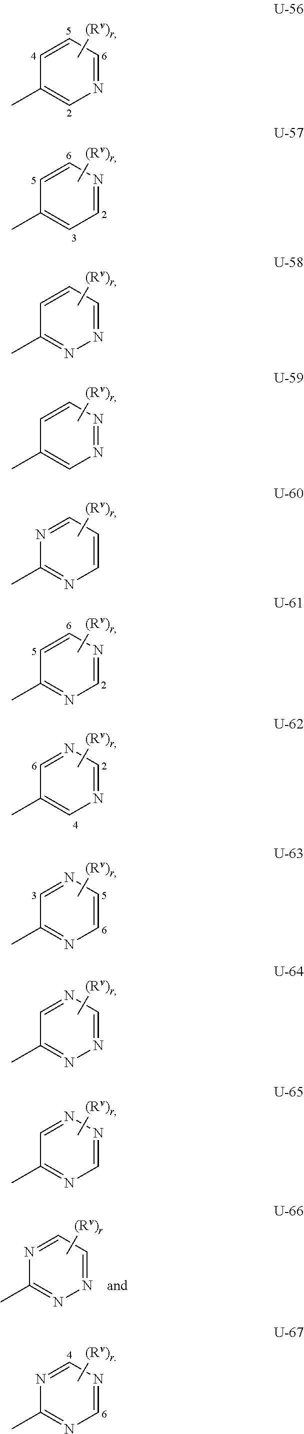

Although R^{v} groups are shown on rings U-1 through U-67, it is noted that they do not need to be present since they are optional substituents. Note that when r is 0, this means the ring is unsubstituted. The nitrogen atoms that require substitution to fill their valence are substituted with H or R^{v}. Note that when the attachment point between (R^{v})ᵣ and the U-ring is illustrated as floating, (R^{v})ᵣ can be attached to any available carbon atom or nitrogen atom of the U-ring.

Examples of a 3-, 4-, 5- or 6-membered saturated or partially unsaturated heterocyclic ring include the rings G-1 through G-45 as illustrated in Exhibit 2 wherein R^{v} is any substituent as defined in the Summary of the Invention for R² or R³ (i.e. R⁶ on carbon ring members and R^{6a} on nitrogen ring members of R², and R⁷ on carbon ring members and R^{7a} on nitrogen ring members of R³) and r is an integer from 0 to 5, limited by the number of available positions on each G-ring. The optional substituents corresponding to (R^{v})ᵣ, can be attached to any available carbon or nitrogen by replacing a hydrogen atom. Note that when the attachment point on the G-ring is illustrated as floating, the G-ring can be attached to the remainder of Formula 1 through any available carbon or nitrogen of the G-ring by replacement of a hydrogen atom.

Note that when R² or R³ comprises a ring selected from G-33, G-34, G-35 and G-41 through G-45, G² is O, S or N. Note that when G² is N, the nitrogen atom can complete its valence by substitution with either H or the substituents corresponding to R^{v} as defined in the Summary of Invention for R² or R³.

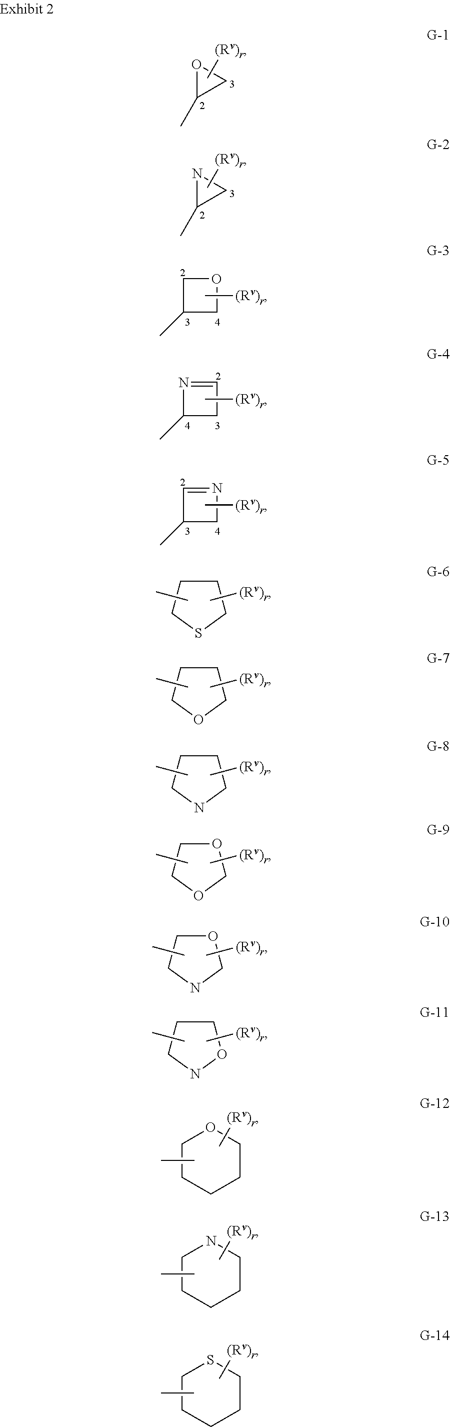

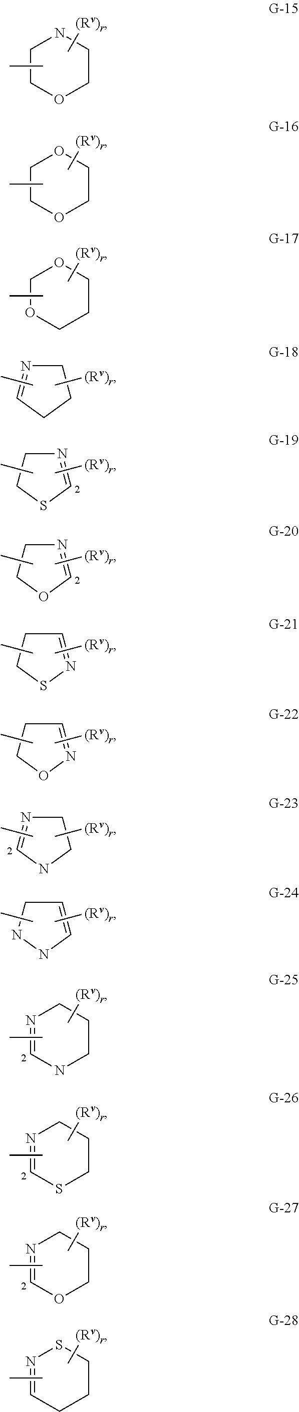

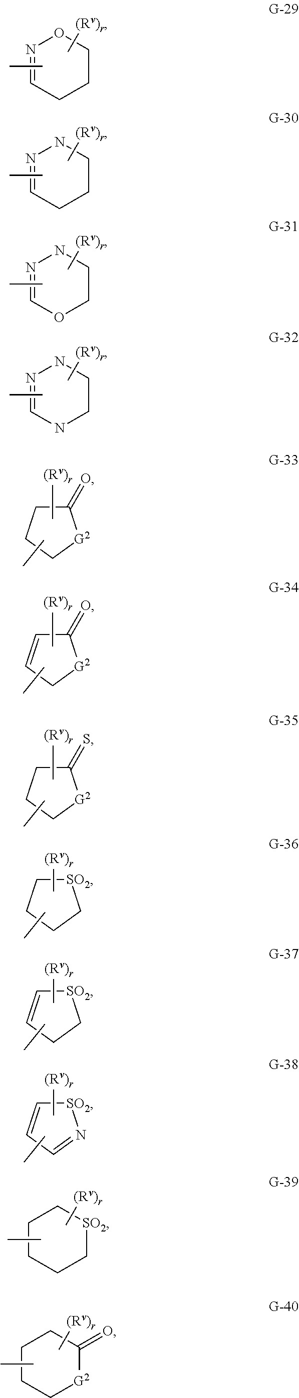

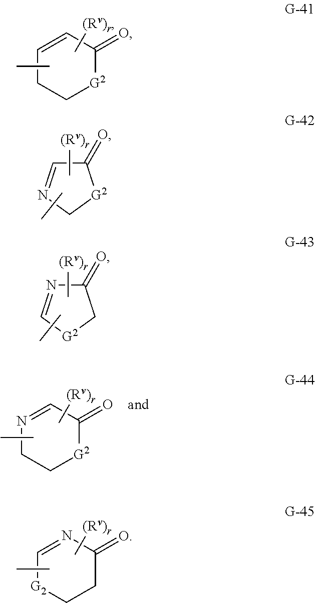

As noted in the Summary of the Invention, when a pair of R⁵ substituents are attached to adjacent ring atoms on the phenyl ring of Formula 1, or when a pair of R⁶ and/or R^{6a} substituents are attached to adjacent ring atoms on the R² ring of Formula 1, or a pair of R⁷ and/or R^{7a} substituents are attached to adjacent ring atoms on the R³ ring of Formula 1, besides the possibility of being separate substituents, they may also be connected to form a ring fused to the respective rings to which they are attached. The fused ring can be a 5-, 6- or 7-membered ring including as ring members the two atoms shared with the ring to which the substituents are attached. The other 3 to 5 ring members of the fused ring are provided by the pair of R⁵ substituents, the pair of R⁶ and/or R^{6a} substituents or the pair of R⁷ and/or R^{7a} substituents taken together. These other ring members can include up to 5 carbon atoms (as allowed by the ring size) and optionally up to 4 heteroatoms selected from up to 2 oxygen, up to 2 sulfur and up to 3 nitrogen. The fused ring is optionally substituted with up to 3 substituents as noted in the Summary of the Invention. Exhibit 3 provides, as illustrative examples, rings formed by a pair of adjacent R⁵, R⁶, R^{6a}, R⁷ or R^{7a} substituents taken together. As these rings are fused with a ring of Formula 1, a portion of the Formula 1 ring is shown and the dashed lines represent the ring bonds of the Formula 1 ring. In certain cases, as illustrated by T-3, T-5, T-8, T-11, T-14 and T-16, the pattern of single and double bonds between ring members in the fused ring may affect the possible patterns of single and double bonds (according to valence bond theory) in the ring it is fused to in Formula 1, but each of the ring member atoms retains sp² hybridized orbitals (i.e. is able to participate in π-bonding). The rings depicted can be fused to any two adjacent atoms of a ring of Formula 1, and furthermore can be fused in either of the two possible orientations. The optional substituents (R^{v})ᵣ, are independently selected from the group consisting of C₁-C₂ alkyl, halogen, cyano, nitro and C₁-C₂ alkoxy on carbon ring members and from the group consisting of C₁-C₂ alkyl, cyano and C₁-C₂ alkoxy on nitrogen ring members. For these T-rings, r is an integer from 0 to 3, limited by the number of available positions on each T-ring. When the attachment point between (R^{v})ᵣ and the T-ring is illustrated as floating, R^{v} may be bonded to any available T-ring carbon or nitrogen atom (as applicable). One skilled in the art recognizes that while r is nominally an integer from 0 to 3, some of the rings shown in Exhibit 3 have less than 3 available positions, and for these groups r is limited to the number of available positions. When “r” is 0 this means the ring is unsubstituted and hydrogen atoms are present at all available positions. If r is 0 and (R^{v})ᵣ is shown attached to a particular atom, then hydrogen is attached to that atom. The nitrogen atoms that require substitution to fill their valence are substituted with H or R^{v}. Furthermore, one skilled in the art recognizes that some of the rings shown in Exhibit 3 can form tautomers, and the particular tautomer depicted is representative of all the possible tautomers.

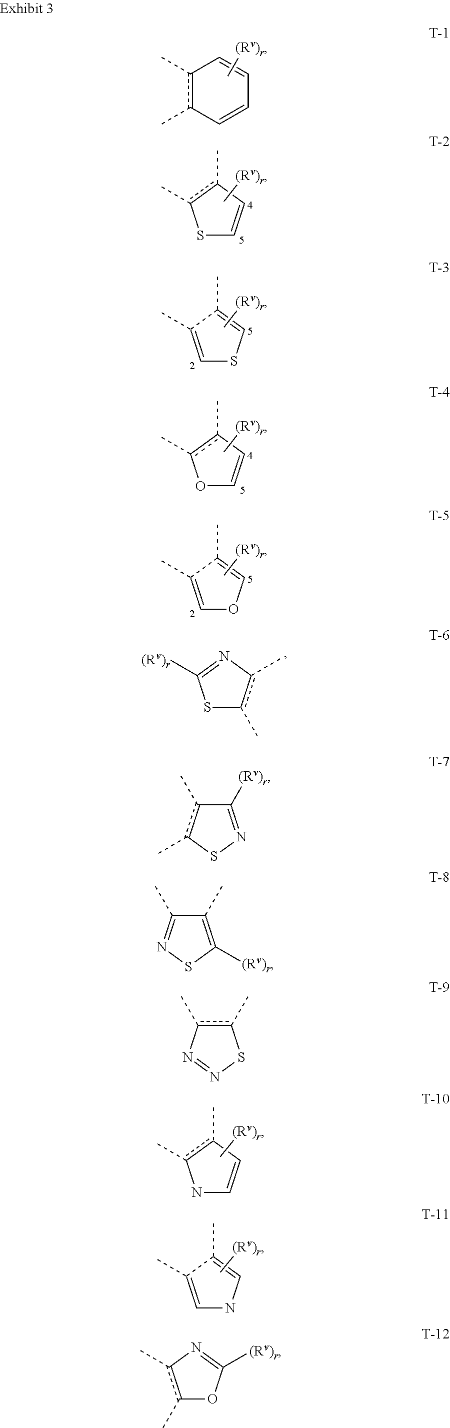

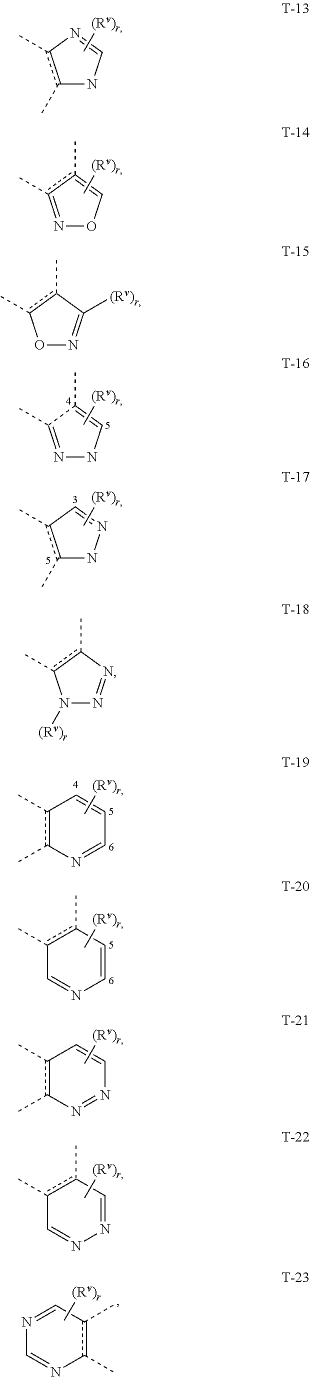

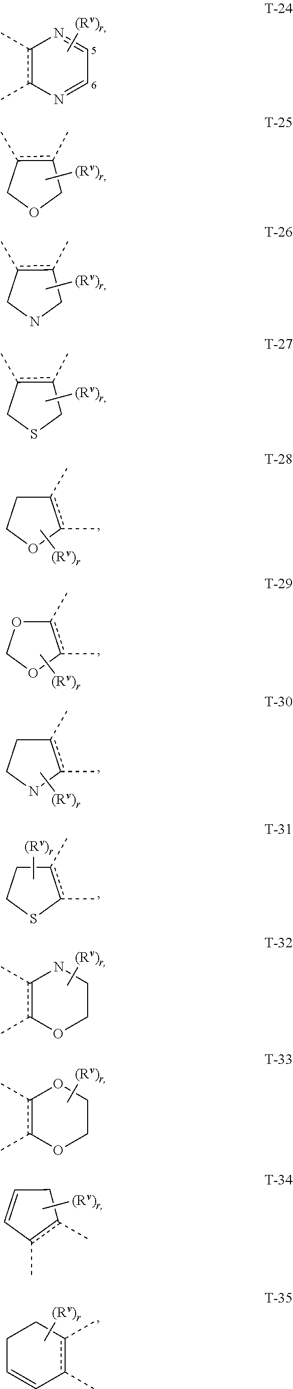

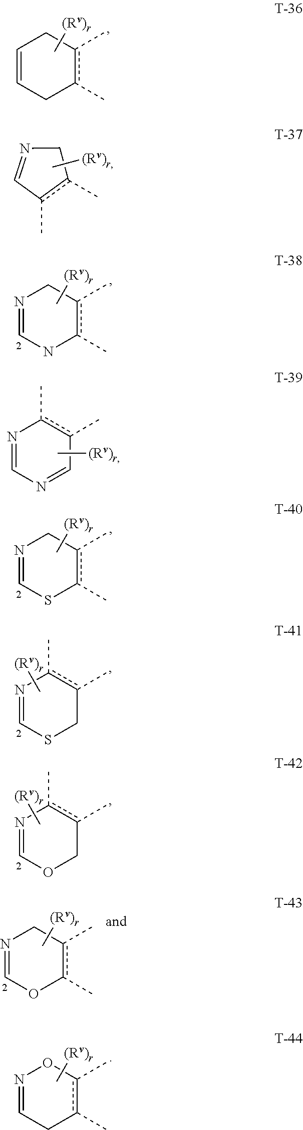

As noted in the Summary of the Invention, a pair of R⁶ or R⁷ substituents, besides the possibility being separate substituents, may also be taken together with the ring atom to which they are attached to form a 5-, 6- or 7-membered spirocyclic ring. The spirocyclic ring includes as a ring member the atom shared with the ring to which the substituents are attached. The other 4 to 6 ring members of the spirocyclic ring are provided by the pair of R⁶ substituents or the pair of R⁷ substituents taken together. Exhibit 4 provides, as illustrative examples, rings formed by a pair of R⁶ or R⁷ substituents being taken together. The dashed lines represent bonds in the ring to which the spirocyclic ring is attached. The optional substituents (R^{v})ᵣ are independently selected from the group consisting of C₁-C₂ alkyl, halogen, cyano, nitro and C₁-C₂ alkoxy on carbon ring members and from the group consisting of C₁-C₂ alkyl, cyano and C₁-C₂ alkoxy on nitrogen ring members. For these J-rings, r is an integer from 0 to 3, limited by the number of available positions on each J-ring. When the attachment point between (R^{v})ᵣ and the J-ring is illustrated as floating, Rv may be bonded to any available J-ring carbon or nitrogen atom. The optional substituents (R^{v})ᵣ, are independently selected from the group consisting of C₁-C₂ alkyl, halogen, cyano, nitro and C₁-C₂ alkoxy on carbon ring members and from the group consisting of C₁-C₂ alkyl, cyano and C₁-C₂ alkoxy on nitrogen ring members. When “r” is 0 this means that the ring is unsubstituted and hydrogen atoms are present at all available positions.

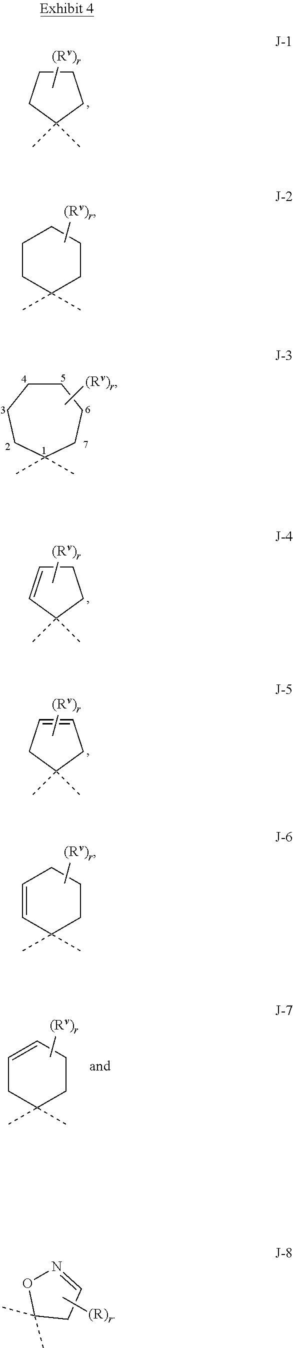

A wide variety of synthetic methods are known in the art to enable preparation of aromatic and nonaromatic heterocyclic rings and ring systems; for extensive reviews see the eight volume set of Comprehensive Heterocyclic Chemistry, A. R. Katritzky and C. W. Rees editors-in-chief, Pergamon Press, Oxford, 1984 and the twelve volume set of Comprehensive Heterocyclic Chemistry II, A. R. Katritzky, C. W. Rees and E. F. V. Scriven editors-in-chief, Pergamon Press, Oxford, 1996.

Compounds of this invention can exist as one or more stereoisomers. The various stereoisomers include enantiomers, diastereomers, atropisomers and geometric isomers. One skilled in the art will appreciate that one stereoisomer may be more active and/or may exhibit beneficial effects when enriched relative to the other stereoisomer(s) or when separated from the other stereoisomer(s). Additionally, the skilled artisan knows how to separate, enrich, and/or to selectively prepare said stereoisomers. The compounds of the invention may be present as a mixture of stereoisomers, individual stereoisomers or as an optically active form.

One skilled in the art will appreciate that not all nitrogen-containing heterocycles can form N-oxides since the nitrogen requires an available lone pair for oxidation to the oxide; one skilled in the art will recognize those nitrogen-containing heterocycles which can form N-oxides. One skilled in the art will also recognize that tertiary amines can form N-oxides. Synthetic methods for the preparation of N-oxides of heterocycles and tertiary amines are very well known by one skilled in the art including the oxidation of heterocycles and tertiary amines with peroxy acids such as peracetic and m-chloroperbenzoic acid (MCPBA), hydrogen peroxide, alkyl hydroperoxides such as t-butyl hydroperoxide, sodium perborate, and dioxiranes such as dimethyldioxirane. These methods for the preparation of N-oxides have been extensively described and reviewed in the literature, see for example: T. L. Gilchrist in Comprehensive Organic Synthesis, vol. 7, pp 748-750, S. V. Ley, Ed., Pergamon Press; M. Tisler and B. Stanovnik in Comprehensive Heterocyclic Chemistry, vol. 3, pp 18-20, A. J. Boulton and A. McKillop, Eds., Pergamon Press; M. R. Grimmett and B. R. T. Keene in Advances in Heterocyclic Chemistry, vol. 43, pp 149-161, A. R. Katritzky, Ed., Academic Press; M. Tisler and B. Stanovnik in Advances in Heterocyclic Chemistry, vol. 9, pp 285-291, A. R. Katritzky and A. J. Boulton, Eds., Academic Press; and G. W. H. Cheeseman and E. S. G. Werstiuk in Advances in Heterocyclic Chemistry, vol. 22, pp 390-392, A. R. Katritzky and A. J. Boulton, Eds., Academic Press.

One skilled in the art recognizes that because in the environment and under physiological conditions salts of chemical compounds are in equilibrium with their corresponding nonsalt forms, salts share the biological utility of the nonsalt forms. Thus a wide variety of salts of the compounds of Formula 1 are useful for control of plant diseases caused by fungal plant pathogens (i.e. are agriculturally suitable). The salts of the compounds of Formula 1 include acid-addition salts with inorganic or organic acids such as hydrobromic, hydrochloric, nitric, phosphoric, sulfuric, acetic, butyric, fumaric, lactic, maleic, malonic, oxalic, propionic, salicylic, tartaric, 4-toluenesulfonic or valeric acids. When a compound of Formula 1 contains an acidic moiety such as phenol, salts also include those formed with organic or inorganic bases such as pyridine, triethylamine or ammonia, or amides, hydrides, hydroxides or carbonates of sodium, potassium, lithium, calcium, magnesium or barium. Accordingly, the present invention comprises compounds selected from Formula 1, N-oxides and agriculturally suitable salts thereof.

Compounds selected from Formula 1, (including all stereoisomers, N-oxides, and salts thereof), typically exist in more than one form, and Formula 1 thus includes all crystalline and non-crystalline forms of the compounds that Formula 1 represents. Non-crystalline forms include embodiments which are solids such as waxes and gums as well as embodiments which are liquids such as solutions and melts. Crystalline forms include embodiments which represent essentially a single crystal type and embodiments which represent a mixture of polymorphs (i.e. different crystalline types). The term “polymorph” refers to a particular crystalline form of a chemical compound that can crystallize in different crystalline forms, these forms having different arrangements and/or conformations of the molecules in the crystal lattice. Although polymorphs can have the same chemical composition, they can also differ in composition due the presence or absence of co-crystallized water or other molecules, which can be weakly or strongly bound in the lattice. Polymorphs can differ in such chemical, physical and biological properties as crystal shape, density, hardness, color, chemical stability, melting point, hygroscopicity, suspensibility, dissolution rate and biological availability. One skilled in the art will appreciate that a polymorph of a compound represented by Formula 1 can exhibit beneficial effects (e.g., suitability for preparation of useful formulations, improved biological performance) relative to another polymorph or a mixture of polymorphs of the same compound represented by Formula 1. Preparation and isolation of a particular polymorph of a compound represented by Formula 1 can be achieved by methods known to those skilled in the art including, for example, crystallization using selected solvents and temperatures.

Embodiments of the present invention as described in the Summary of the Invention include those described below. In the following Embodiments, Formula 1 includes N-oxides and salts thereof, and reference to “a compound of Formula 1” includes the definitions of substituents specified in the Summary of the Invention unless further defined in the Embodiments.
Embodiment 1. A compound of Formula 1 wherein R¹ is halogen, cyano, C₁-C₄ alkyl, C₂-C₄ alkenyl, C₁-C₄ haloalkyl, C₁-C₃ alkoxy, C₁-C₃ haloalkoxy or C₁-C₃ alkylthio.
Embodiment 2. A compound of Embodiment 1 wherein R¹ is halogen, cyano or C₁-C₄ alkyl.
Embodiment 3. A compound of Embodiment 2 wherein R¹ is halogen or C₁-C₂ alkyl.
Embodiment 3a. A compound of Embodiment 3 wherein R¹ is methyl.
Embodiment 4. A compound of Formula 1 or any one of Embodiments 1 through 3a wherein each W and Y is independently CH₂, O, S, NR⁸ or a direct bond.
Embodiment 5. A compound of Formula 1 or any one of Embodiments 1 through 4 wherein each R⁸ is H.
Embodiment 6. A compound of Embodiment 4 wherein each W and Y is independently CH₂, O, S or a direct bond.
Embodiment 7. A compound of Embodiment 6 wherein W is a direct bond.
Embodiment 8. A compound of Embodiment 6 wherein Y is a direct bond.
Embodiment 9. A compound of Formula 1 or any one of Embodiments 1 through 8 wherein R² is a phenyl ring optionally substituted with up to 5 substituents independently selected from R⁶; or a 5- or 6-membered heterocyclic ring containing ring members selected from carbon atoms and up to 4 heteroatoms selected from up to 2 oxygen, up to 2 sulfur and up to 3 nitrogen atoms, wherein up to 3 carbon atom ring members are independently selected from C(═O) and C(═S), and the sulfur atom ring members are independently selected from S(═O)ₚ(═NR⁹)_{q}, the heterocyclic ring optionally substituted with up to 5 substituents selected from R⁶ on carbon atom ring members and Rha on nitrogen atom ring members.
Embodiment 10. A compound of Embodiment 9 wherein R² is a phenyl ring optionally substituted with up to 3 substituents independently selected from R⁶; or a 5- or 6-membered heterocyclic ring containing ring members selected from carbon atoms and up to 4 heteroatoms selected from up to 2 oxygen, up to 2 sulfur and up to 3 nitrogen atoms, wherein up to 3 carbon atom ring members are independently selected from C(═O) and C(═S), and the sulfur atom ring members are independently selected from S(═O)ₚ(═NR⁹)_{q}, the heterocyclic ring optionally substituted with up to 3 substituents selected from R⁶ on carbon atom ring members and R^{6a} on nitrogen atom ring members.
Embodiment 11. A compound of Embodiment 10 wherein R² is a phenyl ring optionally substituted with up to 3 substituents independently selected from R⁶.
Embodiment 12. A compound of Embodiment 11 wherein the R⁶ substituents are at the 2-, 3- and/or 5-positions of the phenyl ring.
Embodiment 13. A compound of Embodiment 11 wherein R² is a phenyl ring optionally substituted with up to 2 substituents independently selected from R⁶.
Embodiment 14. A compound of Embodiment 13 wherein the R⁶ substituents are at the 3- and 5-positions of the phenyl ring.
Embodiment 15. A compound of Embodiment 13 wherein the R⁶ substituents are at the 2- and 5-positions of the phenyl ring.
Embodiment 16. A compound of Formula 1 or any one of Embodiments 1 through 15 wherein R³ is a phenyl ring optionally substituted with up to 5 substituents independently selected from R⁷; or a 5- or 6-membered heterocyclic ring containing ring members selected from carbon atoms and up to 4 heteroatoms selected from up to 2 oxygen, up to 2 sulfur and up to 3 nitrogen atoms, wherein up to 3 carbon atom ring members are independently selected from C(═O) and C(═S), and the sulfur atom ring members are independently selected from S(═O)ₚ(═NR⁹)_{q}, the heterocyclic ring optionally substituted with up to 5 substituents selected from R⁷ on carbon atom ring members and R^{7a} on nitrogen atom ring members; or when is Y is a direct bond, then R³ is also selected from halogen, cyano, C₁-C₆ alkyl, C₂-C₆ alkenyl, C₁-C₆ haloalkyl, C₂-C₆ haloalkenyl, C₃-C₆ cycloalkyl, C₃-C₆ cycloalkenyl, C₂-C₆ alkylcarbonyl, C₂-C₆ alkoxycarbonyl, C₂-C₆ cyanoalkyl and C₁-C₆ hydroxyalkyl.
Embodiment 17. A compound of Embodiment 16 wherein R³ is a phenyl ring optionally substituted with up to 3 substituents independently selected from R⁷; or a 5- or 6-membered heterocyclic ring containing ring members selected from carbon atoms and up to 4 heteroatoms selected from up to 2 oxygen, up to 2 sulfur and up to 3 nitrogen atoms, wherein up to 3 carbon atom ring members are independently selected from C(═O) and C(═S), and the sulfur atom ring members are independently selected from S(═O)ₚ(═NR⁹)_{q}, the heterocyclic ring optionally substituted with up to 3 substituents selected from R⁷ on carbon atom ring members and R^{7a} on nitrogen atom ring members; or when Y is a direct bond, then R³ is also selected from halogen, cyano, C₁-C₆ alkyl, C₂-C₆ alkenyl, C₁-C₆ haloalkyl, C₃-C₆ cycloalkyl, C₂-C₆ alkylcarbonyl, C₂-C₆ alkoxycarbonyl, C₂-C₆ cyanoalkyl and C₁-C₆ hydroxyalkyl.
Embodiment 18. A compound of Embodiment 16 or 17 wherein R³ is an optionally substituted phenyl or heterocyclic ring.
Embodiment 19. A compound of Embodiment 16 or 17 wherein Y is a direct bond and R³ is other than an optionally substituted phenyl or heterocyclic ring.
Embodiment 20. A compound of Embodiment 16 or 17 wherein R³ is a phenyl ring optionally substituted with up to 3 substituents independently selected from R⁷; or when Y is a direct bond, then R³ is also selected from halogen, C₁-C₆ alkyl, C₂-C₆ alkenyl, C₁-C₆ haloalkyl, C₃-C₆ cycloalkyl, C₂-C₆ alkylcarbonyl, C₂-C₆ alkoxycarbonyl, C₂-C₆ cyanoalkyl and C₁-C₆ hydroxyalkyl.
Embodiment 21. A compound of Embodiment 20 wherein when R³ is an optionally substituted phenyl ring, said ring is optionally substituted with up to 1 substituent selected from R⁷.
Embodiment 22. A compound of Embodiment 21 wherein the R⁷ substituent is at the 2- or 3-positions of the phenyl ring.
Embodiment 23. A compound of any one of Embodiments 20 through 22 wherein R³ is an optionally substituted phenyl ring.
Embodiment 24. A compound of any one of Embodiments 20 through 23 wherein when Y is a direct bond, then R³ is also selected from C₁-C₆ alkyl, C₂-C₆ alkenyl, C₁-C₆ haloalkyl, C₃-C₆ cycloalkyl, C₂-C₆ alkylcarbonyl, C₂-C₆ alkoxycarbonyl, C₂-C₆ cyanoalkyl and C₁-C₆ hydroxyalkyl.
Embodiment 25. A compound of Embodiment 24 wherein when Y is a direct bond, then R³ is also selected from halogen, C₁-C₆ alkyl, C₁-C₆ haloalkyl, C₂-C₆ alkenyl, C₂-C₆ cyanoalkyl and C₁-C₆ hydroxyalkyl.
Embodiment 26. A compound of Embodiment 25 wherein when Y is a direct bond, then R³ is also selected from C₁-C₆ alkyl, C₁-C₆ haloalkyl, C₂-C₆ cyanoalkyl and C₁-C₆ hydroxyalkyl.
Embodiment 27. A compound of Embodiment 26 wherein when Y is a direct bond, then R³ is also selected from C₁-C₄ alkyl, C₁-C₃ haloalkyl, C₂-C₄ cyanoalkyl and C₁-C₄ hydroxyalkyl.
Embodiment 28. A compound of any one of Embodiments 20 and 24 through 27 wherein Y is a direct bond, and R³ is other than an optionally substituted phenyl ring.
Embodiment 29. A compound of Formula 1 or any one of Embodiments 1 through 28 wherein R⁴ is H, halogen, hydroxy or C₁-C₂ alkyl.
Embodiment 30. A compound of Embodiment 29 wherein R⁴ is H, halogen or hydroxy.
Embodiment 30a. A compound of Formula 1 or any one of Embodiments 1 through 28 wherein R⁴ is H, cyano or C₁-C₂ alkyl.
Embodiment 31. A compound of Embodiment 30 or 30a wherein R⁴ is H.
Embodiment 32. A compound of Formula 1 or any one of Embodiments 1 through 31 wherein each R⁵, R⁶ and R⁷ is independently halogen, cyano, C₁-C₆ alkyl, C₂-C₆ alkenyl, C₁-C₆ haloalkyl, C₁-C₆ alkoxy, C₁-C₆ haloalkoxy, C₁-C₆ alkylthio or C₁-C₆ haloalkylthio.
Embodiment 33. A compound of Formula 1 or any one of Embodiments 1 through 32 wherein each R⁵, R⁶ and R⁷ is independently halogen, C₁-C₆ alkyl, C₂-C₆ alkenyl, C₁-C₆ haloalkyl or C₁-C₆ alkoxy.
Embodiment 34. A compound of Formula 1 or any one of Embodiments 1 through 33 wherein each R⁵, R⁶ and R⁷ is independently halogen, C₁-C₆ alkyl or C₁-C₆ alkoxy.
Embodiment 35. A compound of Formula 1 or any one of Embodiments 1 through 34 wherein each R⁵, R⁶ and R⁷ is independently halogen, methyl or methoxy.
Embodiment 36. A compound of Formula 1 or any one of Embodiments 1 through 35 wherein each R⁵ is independently halogen or methoxy.
Embodiment 37. A compound of Formula 1 or any one of Embodiments 1 through 36 wherein each R⁶ is independently chlorine or methoxy.
Embodiment 38. A compound of Formula 1 or any one of Embodiments 1 through 37 wherein each R^{6a} and R^{7a} is independently cyano, C₁-C₆ alkyl, C₂-C₆ alkenyl, C₁-C₆ haloalkyl, C₁-C₆ alkoxy, C₁-C₆ haloalkoxy, C₁-C₆ alkylthio or C₁-C₆ haloalkylthio.
Embodiment 39. A compound of Formula 1 or any one of Embodiments 1 through 38 wherein each R^{6a} and R^{7a} is independently C₁-C₆ alkyl, C₂-C₆ alkenyl, C₁-C₆ haloalkyl or C₁-C₆ alkoxy.
Embodiment 40. A compound of Formula 1 or any one of Embodiments 1 through 39 wherein each R^{6a} and R^{7a} is independently C₁-C₆ alkyl.
Embodiment 41. A compound of Formula 1 or any one of Embodiments 1 through 40 wherein when a pair of R⁵ substituents, a pair of R⁶ and/or R^{6a} substituents, and/or a pair of R⁷ and/or R^{7a} substituents attached to adjacent ring atoms are taken together with the atoms to which they are attached to form a fused ring, each fused ring is 5- or 6-membered and contains ring members selected from carbon, and is optionally substituted with up to 3 substituents independently selected from the group consisting of C₁-C₂ alkyl and halogen.
Embodiment 42. A compound of Formula 1 or any one of Embodiments 1 through 41 wherein m is an integer selected from 0, 1, 2 and 3.
Embodiment 43. A compound of Formula 1 or any one of Embodiments 1 through 42 wherein m is 3 and the R⁵ substituents are attached at ortho and para positions.

Embodiments of this invention, including Embodiments 1-43 above as well as any other embodiments described herein, can be combined in any manner, and the descriptions of variables in the embodiments pertain not only to the compounds of Formula 1 but also to the starting compounds and intermediate compounds useful for preparing the compounds of Formula 1. In addition, embodiments of this invention, including Embodiments 1-43 above as well as any other embodiments described herein, and any combination thereof, pertain to the compositions and methods of the present invention.

Combinations of Embodiments 1-43 are illustrated by:

Embodiment A. A compound of Formula 1 wherein
R¹ is halogen, cyano, C₁-C₄ alkyl, C₂-C₄ alkenyl, C₁-C₄ haloalkyl, C₁-C₃ alkoxy, C₁-C₃ haloalkoxy or C₁-C₃ alkylthio;
R² is a phenyl ring optionally substituted with up to 5 substituents independently selected from R⁶; or a 5- or 6-membered heterocyclic ring containing ring members selected from carbon atoms and up to 4 heteroatoms selected from up to 2 oxygen, up to 2 sulfur and up to 3 nitrogen atoms, wherein up to 3 carbon atom ring members are independently selected from C(═O) and C(═S), and the sulfur atom ring members are independently selected from S(═O)ₚ(═NR⁹)_{q}, the heterocyclic ring optionally substituted with up to 5 substituents selected from R⁶ on carbon atom ring members and R^{6a} on nitrogen atom ring members;
R³ is a phenyl ring optionally substituted with up to 5 substituents independently selected from R⁷; or a 5- or 6-membered heterocyclic ring containing ring members selected from carbon atoms and up to 4 heteroatoms selected from up to 2 oxygen, up to 2 sulfur and up to 3 nitrogen atoms, wherein up to 3 carbon atom ring members are independently selected from C(═O) and C(═S), and the sulfur atom ring members are independently selected from S(═O)ₚ(═NR⁹)_{q}, the heterocyclic ring optionally substituted with up to 5 substituents selected from R⁷ on carbon atom ring members and R^{7a} on nitrogen atom ring members; or
when Y is a direct bond, then R³ is also selected from halogen, cyano, C₁-C₆ alkyl, C₂-C₆ alkenyl, C₁-C₆ haloalkyl, C₂-C₆ haloalkenyl, C₃-C₆ cycloalkyl, C₂-C₆ alkylcarbonyl, C₂-C₆ alkoxycarbonyl, C₂-C₆ cyanoalkyl and C₁-C₆ hydroxyalkyl;
each R⁵, R⁶ and R⁷ is independently halogen, cyano, C₁-C₆ alkyl, C₂-C₆ alkenyl, C₁-C₆ haloalkyl, C₁-C₆ alkoxy, C₁-C₆ haloalkoxy, C₁-C₆ alkylthio or C₁-C₆ haloalkylthio; and
each R^{6a} and R^{7a} is independently cyano, C₁-C₆ alkyl, C₂-C₆ alkenyl, C₁-C₆ haloalkyl, C₁-C₆ alkoxy, C₁-C₆ haloalkoxy, C₁-C₆ alkylthio or C₁-C₆ haloalkylthio.

Embodiment B. A compound of Embodiment A wherein
R¹ is halogen, cyano or C₁-C₄ alkyl;
each W and Y is independently CH₂, O, S or a direct bond;
R² is a phenyl ring optionally substituted with up to 3 substituents independently selected from R⁶; or a 5- or 6-membered heterocyclic ring containing ring members selected from carbon atoms and up to 4 heteroatoms selected from up to 2 oxygen, up to 2 sulfur and up to 3 nitrogen atoms, wherein up to 3 carbon atom ring members are independently selected from C(═O) and C(═S), and the sulfur atom ring members are independently selected from S(═O)ₚ(═NR⁹)_{q}, the heterocyclic ring optionally substituted with up to 3 substituents selected from R⁶ on carbon atom ring members and R^{6a} on nitrogen atom ring members; and
R³ is a phenyl ring optionally substituted with up to 3 substituents independently selected from R⁷; or a 5- or 6-membered heterocyclic ring containing ring members selected from carbon atoms and up to 4 heteroatoms selected from up to 2 oxygen, up to 2 sulfur and up to 3 nitrogen atoms, wherein up to 3 carbon atom ring members are independently selected from C(═O) and C(═S), and the sulfur atom ring members are independently selected from S(═O)ₚ(═NR⁹)_{q}, the heterocyclic ring optionally substituted with up to 3 substituents selected from R⁷ on carbon atom ring members and R^{7a} on nitrogen atom ring members; or
when Y is a direct bond, then R³ is also selected from C₁-C₆ alkyl, C₂-C₆ alkenyl, C₁-C₆ haloalkyl, C₃-C₆ cycloalkyl, C₂-C₆ alkylcarbonyl, C₂-C₆ alkoxycarbonyl, C₂-C₆ cyanoalkyl and C₁-C₆ hydroxyalkyl.

Embodiment C. A compound of Embodiment B wherein
R¹ is halogen or C₁-C₂ alkyl;
W is a direct bond;
Y is a direct bond;
R² is a phenyl ring optionally substituted with up to 3 substituents independently selected from R⁶;
R³ is a phenyl ring optionally substituted with up to 3 substituents independently selected from R⁷; or
R³ is C₁-C₆ alkyl, C₂-C₆ alkenyl, C₁-C₆ haloalkyl, C₂-C₆ cyanoalkyl or C₁-C₆ hydroxyalkyl;
R⁴ is H, cyano or C₁-C₂ alkyl;
each R⁵, R⁶ and R⁷ is independently halogen, C₁-C₆ alkyl, C₂-C₆ alkenyl, C₁-C₆ haloalkyl or C₁-C₆ alkoxy; and
each R^{6a} and R^{7a} is independently C₁-C₆ alkyl, C₂-C₆ alkenyl, C₁-C₆ haloalkyl or C₁-C₆ alkoxy.

Embodiment D. A compound of Embodiment C wherein
R¹ is methyl;
R² is a phenyl ring optionally substituted with up to 2 substituents independently selected from R⁶;
R³ is a phenyl ring optionally substituted with up to 1 substituent selected from R⁷; or
R³ is C₁-C₄ alkyl, C₁-C₃ haloalkyl, C₂-C₄ cyanoalkyl and C₁-C₄ hydroxyalkyl;
R⁴ is H;
each R⁵, R⁶ and R⁷ is independently halogen, C₁-C₆ alkyl or C₁-C₆ alkoxy;
each R^{6a} and R^{7a} is independently C₁-C₆ alkyl; and
m is an integer selected from 0, 1, 2 and 3.

Embodiment E. A compound of Embodiment D wherein
each R⁵ is independently halogen or methoxy; and
each R⁶ is independently chlorine or methoxy.

Specific embodiments include compounds of Formula 1 selected from the group consisting of:
4-(3,5-dimethoxyphenyl)-6-methyl-5-(2,4,6-trifluorophenyl)-3-pyridineacetonitrile,
4-(3,5-dimethoxyphenyl)-5-(2-fluorophenyl)-2-methyl-3-(2,4,6-trifluorophenyl)pyridine,
5-(2,6-difluoro-4-methoxyphenyl)-4-(3,5-dimethoxyphenyl)-α,α,6-trimethyl-3-pyridinemethanol,
5-(chloromethyl)-4-(3,5-dimethoxyphenyl)-2-methyl-3-(2,4,6-trifluorophenyl)pyridine,
4-(3,5-dimethoxyphenyl)-2-methyl-5-phenyl-3-(2,4,6-trifluorophenyl)pyridine,
4-(2-chloro-3,5-dimethoxyphenyl)-6-methyl-5-(2,4,6-trifluorophenyl)-3-pyridineacetonitrile,
4-(2-chloro-3,5-dimethoxyphenyl)-5-(2-fluorophenyl)-2-methyl-3-(2,4,6-trifluorophenyl)pyridine, and
4-(3,5-dimethoxyphenyl)-5-ethyl-2-methyl-3-(2,4,6-trifluorophenyl)pyridine.

This invention provides a fungicidal composition comprising a compound of Formula 1 (including all stereoisomers, N-oxides, and salts thereof), and at least one other fungicide. Of note as embodiments of such compositions are compositions comprising a compound corresponding to any of the compound embodiments described above.

This invention provides a fungicidal composition comprising a compound of Formula 1 (including all stereoisomers, N-oxides, and salts thereof) (i.e. in a fungicidally effective amount), and at least one additional component selected from the group consisting of surfactants, solid diluents and liquid diluents. Of note as embodiments of such compositions are compositions comprising a compound corresponding to any of the compound embodiments described above.

This invention provides a method for controlling plant diseases caused by fungal plant pathogens comprising applying to the plant or portion thereof, or to the plant seed, a fungicidally effective amount of a compound of Formula 1 (including all stereoisomers, N-oxides, and salts thereof). Of note as embodiment of such methods are methods comprising applying a fungicidally effective amount of a compound corresponding to any of the compound embodiments describe above. Of particular notes are embodiment where the compounds are applied as compositions of this invention.

One or more of the following methods and variations as described in Schemes 1-21 can be used to prepare the compounds of Formula 1. The definitions of R¹, R², R³, R⁴, R⁵, R⁷, W and Y in the compounds of Formulae 1-27 below are as defined above in the Summary of the Invention unless otherwise noted. Compounds of Formulae 1a-1f are various subsets of the compounds of Formula 1, and all substituents for Formulae 1a-1f are as defined above for Formula 1. Formulae 2a and 2b are subsets of Formula 2.

As shown in Scheme 1, compounds of Formula 1 can be synthesized from compounds of Formula 2 wherein Lg is a leaving group such as halogen (e.g., Cl, Br, I), sulfonate (e.g., OS(O)₂CH₃, OS(O)₂CF₃, OS(O)₂Ph-p-CH₃), and the like, using various coupling reagents in conjunction with a transition metal catalyst. In particular, compounds of Formula 2 can be contacted with compounds of Formula 3 in the presence of a palladium, copper, nickel or iron catalyst to produce compounds of Formula 1 wherein W is CH₂ or a direct bond and R² is an optionally substituted phenyl or heterocyclic ring bonded through carbon. In this method compounds of Formula 3 are organoboronic acids (e.g., M¹ is B(OH)₂), organotrifluoroborates (e.g., M¹ is BF₃K), organoboronic esters (e.g., M¹ is B(—OC(CH₃)₂C(CH₃)₂O—)), organotin reagents (e.g., M¹ is Sn(n-Bu)₃, Sn(Me)₃), Grignard reagents (e.g., M¹ is MgX¹) or organozinc reagents (e.g., M¹ is ZnX¹) wherein X¹ is Br or Cl. Suitable transition metal catalysts include but are not limited to palladium(II) acetate, palladium(II) chloride, tetrakis(triphenylphosphine)palladium(0), bis(triphenylphosphine)-palladium(II) dichloride, dichloro [1,1′-bis (diphenylphosphino)-ferrocene]palladium(II), bis(triphenylphosphine)dichloronickel(II), copper(I) salts (e.g., copper(I) iodide, copper(I) bromide, copper(I) chloride, copper(I) cyanide and copper(I) triflate) and iron(III) acetylacetonate. Optimal conditions for each reaction will depend upon the catalyst used and the counterion attached to the compound of Formula 3 (i.e. M¹), as is understood by one skilled in the art. In some cases the addition of a ligand such as a substituted phosphine or a substituted bisphosphinoalkane promotes reactivity. Also, the presence of a base (such as an alkali carbonate, tertiary amine or alkali fluoride) is typically necessary for reactions involving compounds of Formula 3 that are boronic acids or organotrifluoroborates. For reviews of this type of reaction see: E. Negishi, Handbook of Organopalladium Chemistry for Organic Synthesis, John Wiley and Sons, Inc., New York, 2002; N. Miyaura, Cross-Coupling Reactions: A Practical Guide, Springer, New York, 2002; H. C. Brown et al., Organic Synthesis via Boranes, Vol. 3, Aldrich Chemical Co., Milwaukee, Wis., 2002; Suzuki et al., Chemical Review 1995, 95, 2457-2483 and Molander et al., Accounts of Chemical Research 2007, 40, 275-286. Also, Step D of Example 1 illustrates the synthesis of a compound of Formula 1 wherein W is a direct bond and R² is a substituted phenyl ring.

Compounds of Formula 1 wherein W is C(═O) can be prepared from compounds of Formulae 2 and 3 by means of a carbonylative cross-coupling reaction. In this method M¹ is typically B(OH)₂, Sn(n-Bu)₃, Sn(Me)₃, MgX¹ or ZnX¹. The reaction is usually run at using carbon monoxide at about 100-1000 kPa pressure in the presence of a palladium, copper or nickel catalyst in a mixture of an alcohol and another solvent such as N,N-dimethylformamide, N-methylpyrrolidinone or tetrahydrofuran, or mixtures of acetone and N,N-dimethylformamide at temperatures ranging from about room temperature (e.g., 20° C.) to 150° C. For references describing this method see, for example, Brunet et al., Chemical Society Reviews 1995, 24(2), 89-95; Kollar et al., Current Organic Chemistry 2002, 6(12), 1097-1119; and Suzuki et al., Chemical Review 1995, 95, 2457-2483.

Compounds of Formula 1 wherein W is a direct bond and R² is a N-linked heterocyclic ring, or W is O, S, NR⁸ can be prepared via a cross-coupling reaction of compounds of Formula 2 and Formula 4. Typical reaction conditions involve the presence of a base (e.g., NaOt-Bu, K₂CO₃, K₃PO₄ or Cs₂CO₃), a palladium, nickel or copper catalyst (e.g., Pd₂(dba)₃, Pd(OAc)₂, Ni(COD)₂, CuI) and optionally a ligand (e.g., DPPF, DPPP, BINAP, BINOL or 1,1,1-tris(hydroxymethyl)ethane). For relevant literature references see, for example, Chen et al., Organic Letters 2006, 8, 5609-5612; Hartwig, Angew. Chem. Int. Ed. 1998, 37(15), 2046-2067; and Buchwald et al., Accounts of Chemical Research 1998, 31(12), 805-818.

Alternatively, compounds of Formula 1 wherein W is O, S or NR⁸ can be prepared from compounds of Formulae 2 and 3 wherein M¹ is Na or K (formed by treating the corresponding alcohol, thiol or amine with base). Typical reaction conditions involve running the reaction in the presence of a palladium or nickel catalyst (e.g., Pd(dba)₂, Pd(Ph₃)₄, Ni(COD)₂) and optionally a ligand (e.g., DPPP, BINOL) and optionally a base (e.g., NaH), in a solvent such as toluene or DMF. In some cases compounds of Formula 1 can also be obtained from uncatalyzed reactions of Formulae 2 and 3 (wherein M¹ is Na or K); however these reactions typically involve harsher conditions and longer reaction times. For leading references see, for example, Buchwald et al., Metal-Catalyzed Cross-Coupling Reactions, 2nd Edition, Wiley-VCH, Germany, 2004, 699-760; Hartwig, Angew. Chem. Int. Ed. 1998, 37(15), 2046-2067; and references cited therein.

One skilled in the art will appreciate that the leaving group Lg attached to compounds of Formula 2 should be selected in view of the relative reactivity of other functional groups present on Formula 2 (i.e. R¹, YR³ and R⁴), so that the group Lg is displaced and not the functional groups to give the final compounds of Formula 1. Depending on the functional groups attached to Formula 2, alternative methods my be desirable to prepare compounds of Formula 1, such as the methods discussed regarding other Schemes below.

Compounds of Formulae 3 and 4 are commercially available or can be prepared by a wide variety of general methods known in the art.

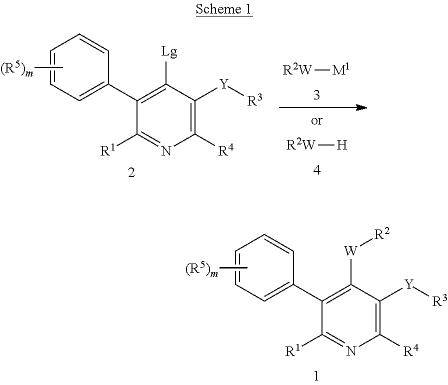

As shown in Scheme 2, compounds of Formula 2 can be prepared by a regioselective metal-catalyzed cross-coupling reaction. Selective introduction of YR³ to give compounds of Formula 2 can be achieved by treating intermediates of Formula 5 wherein X² is halogen (e.g., Cl, Br or I). For optimal selectivity (i.e. preferential displacement of X² to give a Formula 2 compound), the Lg group should be less reactive than X² under cross-coupling conditions, thus allowing for differentiation between the two reactive centers. For example, use of compounds of Formula 5 wherein X² is Br or I and Lg is Cl often provides optimal selectivity. Compounds of Formula 2 wherein Y is CH₂ or a direct bond and R³ is an optionally substituted phenyl ring or heterocyclic ring bonded through carbon, or Y is a direct bond and R³ is alkyl, alkenyl, haloalkenyl, alkynyl, haloalkynyl, and the like, can be prepared by reacting a pyridine of Formula 5 with an organometallic compound of Formula 6 analogous to the method described in Scheme 1. M¹ is as described for the method of Scheme 1. Examples of reactions of this type can be found in Czarnocki et al., Synthesis 2006, 17, 2855-2864; Friesen et al., Bioorganic & Medicinal Chemistry Letters 1998, 8, 2777-2782; Godard et al., Tetrahedron 1992, 48(20), 4123-4134 and Spivey et al., Journal of Organic Chemistry 2003, 68, 7379-7385.

Compounds of Formula 2 wherein Y is a direct bond and R³ is a N-linked heterocyclic ring, alkoxy, alkylsulfinyl, alkylsulfonyl, and the like, or Y is O, S, NR⁸ can be prepared by contacting compounds of Formulae 5 and 7 using the conditions described in Scheme 1. Compounds of Formula 2 wherein R³ is —CHO, alkoxycarbonyl, and the like, can be prepared by a carbonylation reaction also as described in Scheme 1. Displacement of X² by cyanide using methods known in the literature provides compounds of Formula 2 wherein Y is a direct bond and R³ is cyano. These methods include the use of a cyanide salt, usually employing a nickel or palladium catalyst, and often in the presence of a ligand such as a substituted phosphine. Suitable methods include those described by Maligres et al., Tetrahedron Letters 1999, 40, 8193-8195; Beller et al., Chemical European Journal 2003, 9(8), 1828-1836; Buchwald, Journal of the American Chem. Soc. 2003, 125, 2890-2891; Arvela et al., J. Org. Chem. 2003, 68, 9122-9125. One versed in the art will appreciate that when R¹ and/or R⁴ are C¹, X² of Formula 5 is preferably Br or I to obtain optimal selectivity in the method of Scheme 2.

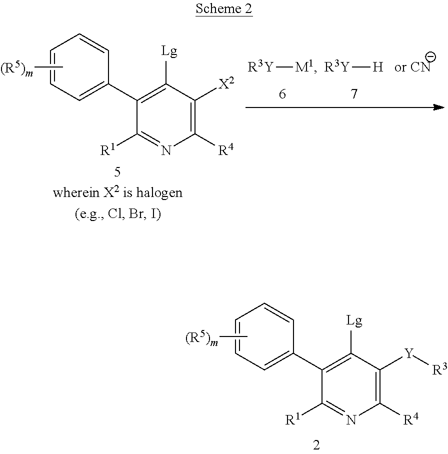

Compounds of Formula 5 wherein Lg is halogen can be prepared from corresponding pyridones of Formula 8 as shown in Scheme 3. Treatment of a compound of Formula 8 with a halogenating reagent affords the halo compound of Formula 5. Suitable halogenating reagents for this method include phosphorus oxyhalides, phosphorus trihalides, phosphorus pentahalides, thionyl chloride, oxalyl chloride, phenylphosphonic dichloride, phosgene and sulfur tetrafluoride. Phosphorus oxyhalides and phosphorus pentahalides are particularly useful. Suitable solvents for this reaction include, for example, dichloromethane, chloroform, chlorobutane, benzene, xylenes, chlorobenzene, tetrahydrofuran, p-dioxane, acetonitrile, and the like. In many cases the reaction can be carried out without solvent other than the compound of Formula 8 and the halogenating reagent. Optionally, an organic base, such as triethylamine, pyridine, N,N-dimethylaniline, and the like, can be added. Addition of a catalyst, such as N,N-dimethylformamide, is also an option. Typical reaction temperatures range from about room temperature (e.g., 20° C.) to 200° C. For representative procedures see Czarnocki et al., Synthesis 2006, 17, 2855-2864; Mphahlele et al., Journal of the Chem. Society, Perkin Trans. 2 2002, 2159-2164; and Albert et al., Journal Chem. Soc. 1964, 1666-1673. The method of Scheme 3 is illustrated in Step D of Example 5.

Compounds of Formula 5 wherein Lg is a sulfonate (e.g., OS(O)₂CH₃, OS(O)₂CF₃, OS(O)₂Ph-p-CH₃) can also be prepared from pyridones of Formula 8 by treatment with a sulfonating reagent such as methanesulfonyl chloride, p-toluenesulfonyl chloride, trifluoromethanesulfonic anhydride or N-phenyltrifluoromethanesulfonimide. The reaction is typically run in the presence of a solvent and a base. Suitable solvents include dichloromethane, tetrahydrofuran, acetonitrile, and the like. Suitable bases include tertiary amines (e.g., triethylamine, N,N-diisopropylethylamine) and potassium carbonate. The reaction is typically conducted at a temperature between about −50° C. and the boiling point of the solvent. For references describing this general method see, for example, Martin et al., Tetrahedron Letters 1993, 34(14), 2235-2238; Kuo et al., Journal of Medicinal Chemistry 1993, 36, 1146-1156; Potts et al., Journal of Organic Chemistry 1991, 56, 4815-4816; and Godard et al., Tetrahedron 1992, 48(20), 4123-4134.

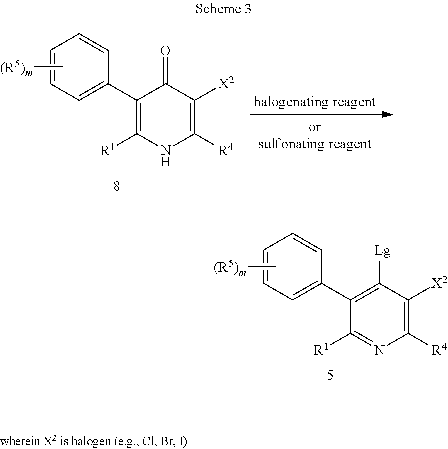

As shown in Scheme 4, halogenation of compounds of Formula 9 provides compounds of Formula 8. Suitable halogenating reagents include elemental halogens (chlorine, bromine, or iodine), N-chlorosuccinimide (NCS), N-bromosuccinimide (NBS) or N-iodosuccinimide (NIS). Solvents used in this reaction are preferably inert to the halogenating conditions, and include, for example, dichloromethane, 1,2-dichloroethane, chloroform, methanol, ethanol, isopropanol, N,N-dimethylformamide, N,N-dimethylacetamide and acetic acid. The method of Scheme 4 can be conducted over a wide range of temperatures, typically from about 0 to 100° C., with optimal temperatures depending upon the reagents employed. Halogenation reactions of this type are well known in the literature; for example see Wojtasiewicz et al., Synthesis 2006, 17, 2855-2864 and Bradbury et al., Journal of Medicinal Chemistry 1993, 36, 1245-54. Also, the method of Scheme 4 is illustrated in Step C of Example 5.

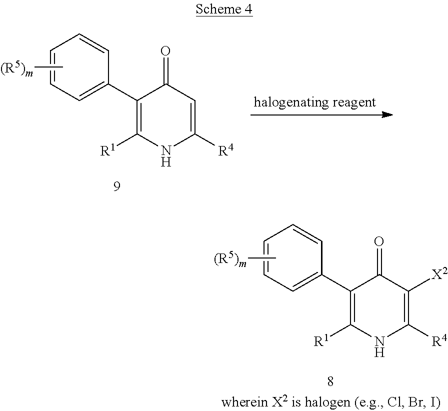

Compounds of Formula 9 can be prepared from compounds of Formula 10 by reaction with ammonia as illustrated in Scheme 5. The ammonia can be supplied as a gas or concentrated solution in a solvent (e.g., ammonium hydroxide), or the ammonia can be formed in situ by contact of ammonium salts (e.g., ammonium chloride, ammonium sulfate or ammonium acetate) with bases. For representative procedures see, for example, Proctor et al., Journal of Pharmaceutical Sciences 1980, 69(9), 1074-1076; and Wojtasiewicz et al., Synthesis 2006, 17, 2855-2864. Also, Step B of Example 5 illustrates the method of Scheme 5.

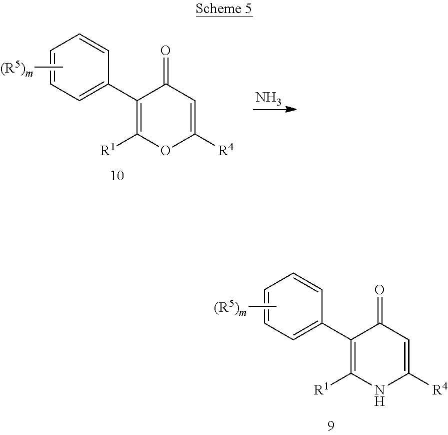

Compounds of Formula 10 are either commercially available or can be prepared by known methods, for example, see Lopez et al., Tetrahedron Letters 2007, 48, 2063-2065 and Tyvorskii et al., Tetrahedron 2000, 56, 7313-7318. Also, Step A of Example 5 illustrates the preparation of a compound of Formula 10.

An alternative method to Scheme 2 for preparing compounds of Formula 2a (Formula 2 wherein Y is a direct bond, R³ is a phenyl ring optionally substituted with R⁷ and Lg is Cl) wherein (R⁷)ⱼ is the same as (R⁵)ₘ is shown in Scheme 6. In this method a compound of Formula 11 (wherein X³ is a leaving group such as Br or I) is treated with at least 2 equivalents of a compound of Formula 12, using conditions analogous to Scheme 2. M¹ is as described for the methods of Schemes 1 and 2. In view of the susceptibility of the Cl group in compounds of Formula 11 to also be displaced, optimal selectivity is achieved when X³ is a stronger leaving group relative to Cl. Generally, suitable choices for X³ include Br or I. The R⁵ and R⁷ substituents shown on Formula 2a, when present, are attached at the corresponding positions on the respective phenyl rings. Examples of reactions of this type can be found in Wojtasiewicz et al., Synthesis 2006, 17, 2855-2864.

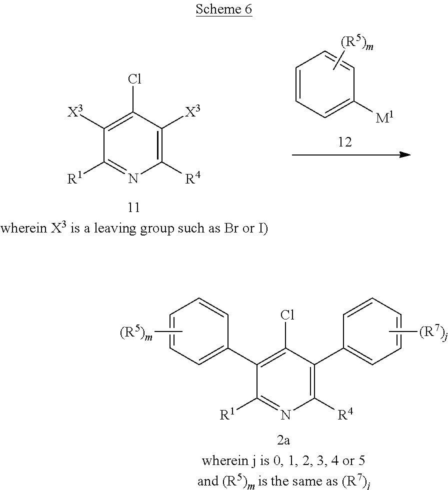

Compounds of Formula 2b (Formula 2 wherein Y is a direct bond and R³ is alkoxycarbonyl) can be prepared as depicted in Scheme 7. Of note is use of this method for preparing compounds of Formula 2b wherein Lg is Cl or Br by treatment of a compound of Formula 13 with phosphorus oxychloride or phosphorus oxybromide. The method of Scheme 7 is illustrated in Step C of Example 1.

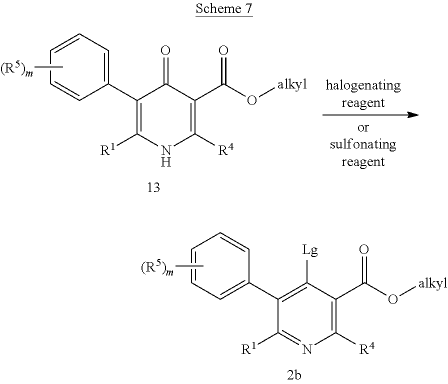

Compounds of Formula 13 can be synthesized by methods well documented in the chemical literature; for example, see Blackaby et al., Bioorganic & Medicinal Chemistry Letters 2005, 15(22), 4998-5002; Kawasaki et al., Journal of Heterocyclic Chemistry 1977, 14(3), 477-82; Kappe et al., Zeitschrift fuer Naturforschung, Teil B 1980, 35B, 892-5; and PCT Publication WO 1999/48892. Also, the preparation of a compound of Formula 13 is illustrated in Step B of Example 1.

Compounds of Formula 2b are useful intermediates for preparing compounds of Formulae 1a (Formula 1 wherein Y is direct bond and R³ is alkylcarbonyl) and 1b (Formula 1 wherein Y is a direct bond and R³ is hydroxyalkyl). The method involves a two-step synthesis as outlined in Scheme 8, which results in a mixture of compounds of Formulae 1a and 1b. In the first step a compound of Formula 14 is prepared via a Pd-catalyzed cross coupling reaction of a compound of Formula 2b with an optionally substituted phenyl- or heterocyclic-boronic acid of Formula 3 where M¹ is B(OH)₂ using the method described in Scheme 1. Subsequent treatment of Formula 14 with an alkyl Grignard reagent in a suitable solvent such as tetrahydrofuran, ether or toluene results in a mixture of compounds of Formulae 1a and 1b, which can be separated by standard techniques known to those skilled in the art. Reactions of this type can be found in the literature; see, for example, Cooke, Journal of Organic Chemistry 1986, 51(6) 951-953. The present Example 2 illustrates the method of Scheme 8.

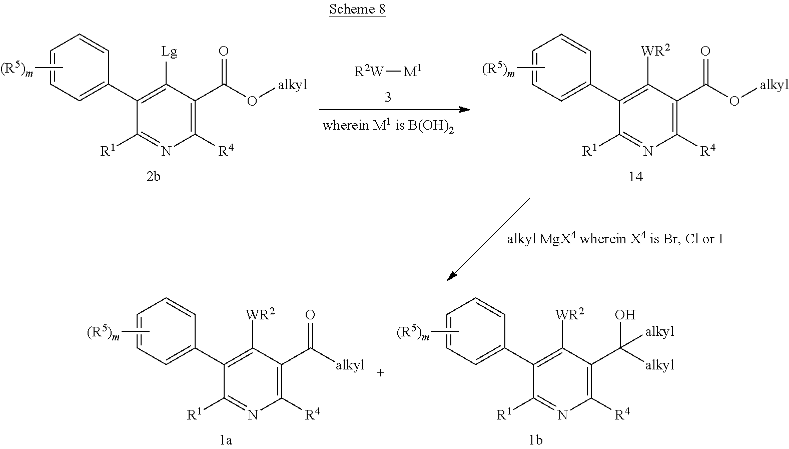

As shown in Scheme 9, compounds of Formula 1b are useful intermediates for preparing compounds of Formula 1c (Formula 1 wherein Y is direct bond and R³ is alkenyl). Dehydration of tertiary alcohols to provide α-substituted styrene derivatives using acidic conditions (e.g., p-toluenesulfonic acid, acetic/sulfuric acid) is well known in the literature; see, for example, Schirok, Journal of Organic Chemistry 2006, 71(15) 5538-5545. The present Example 3 illustrates this method. Subsequent reduction of Formula 1c using catalytic hydrogenation conditions (e.g., palladium on carbon in methanol under hydrogen) provides compounds of Formula 1d (Formula 1 wherein Y is a direct bond and R³ alkyl). The chemical literature disclosing this type of reduction is extensive; see, for example, Catalytic Hydrogenation, L. Cerveny, Ed., Elsevier Science, Amsterdam, 1986. One skilled in the art will recognize that certain functionalities that may be present in compounds of Formula 1c are susceptible to reduction under catalytic hydrogenation conditions (e.g., when R⁴ is halogen), requiring a suitable choice of catalyst and conditions. The hydrogenation method of Scheme 9 is illustrated in Example 4.

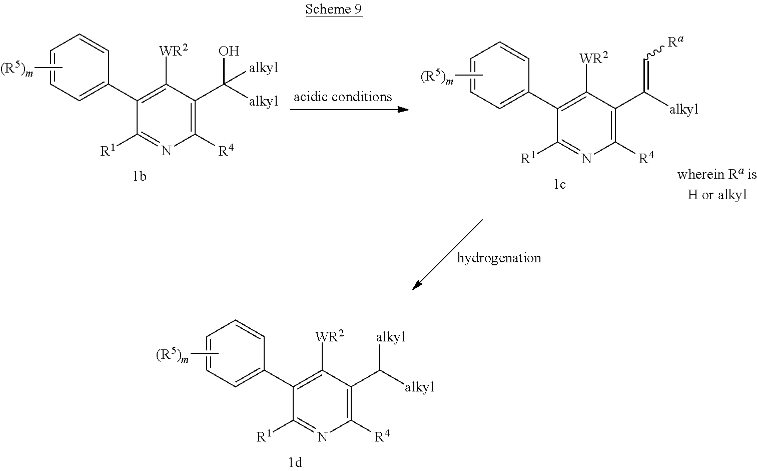

As depicted in Scheme 10, using catalytic hydrogenation conditions analogous to Scheme 9 (e.g., palladium on carbon in methanol under hydrogen), compounds of Formula 1e (Formula 1 wherein W and Y are both a direct bond and R⁴ is halogen) can be reduced to provide compounds of Formula if wherein R⁴ is H. The method of Scheme 10 is illustrated in Example 8.

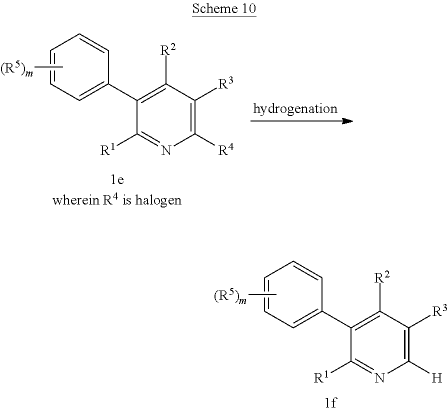

Compounds of Formula 1e can be prepared by the method of Scheme 1 or Scheme 9, or from compounds of Formula 16 by treatment with a halogenating reagent as shown in Scheme 11. Suitable halogenating reagents for this method include phosphorus oxyhalides, phosphorus trihalides, phosphorus pentahalides, thionyl chloride, oxalyl chloride, phenylphosphonic dichloride, phosgene and sulfur tetrafluoride. Preferred are phosphorus oxyhalides and phosphorus pentahalides. Particularly useful for chlorination is phosphorus oxychloride or phenylphosphonic dichloride. The reaction can be run without solvent or in a variety of solvents (e.g., dichloromethane, chloroform, chlorobutane, benzene, xylenes, chlorobenzene, tetrahydrofuran, p-dioxane, acetonitrile) at temperatures ranging from about 70 to 250° C. The method of Scheme 11 is illustrated in Step C of Example 7. Compounds of Formula 16 are tautomers of compounds of Formula 1 where R⁴ is hydroxy. These compounds are particularly useful as intermediates and do not show strong fungicidal activity.

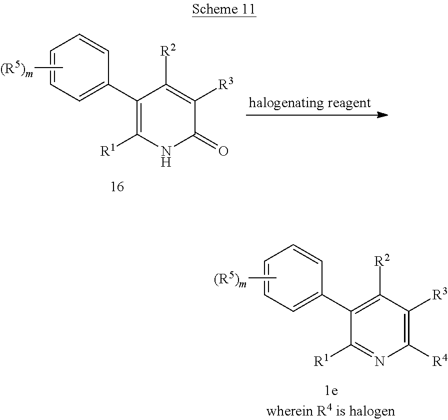

Compounds of Formula 16 can be prepared by reaction of compounds of Formulae 17 and 18, as shown in Scheme 12. The reaction is typically run under acidic conditions employing reagents such sulfuric acid or polyphosphoric acid at temperatures ranging from about room temperature (e.g., 20° C.) to 150° C. Examples of reactions of this type can be found in Carabateas et al., Journal of Heterocyclic Chemistry 1984, 21, 1849-56; Hauser et al., Journal of the American Chemical Society 1957, 79, 728-731 and Wajon et al., Recueil des Travaux Chimiques des Pays-Bas et de la Belgique 1957, 76, 65-74. The method of Scheme 12 is illustrated in Step B of Example 7.

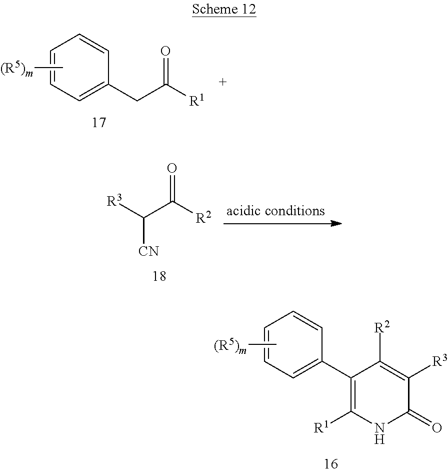

Compounds of Formula 17 are commercially available or readily prepared by methods known to one skilled in the art. Compounds of Formula 18 can be prepared by acylation of nitriles of Formula 19 with esters of Formula 20 in the presence of a base as shown in Scheme 13. Reactions of this type are in known in the art; for a particularly convenient method see Vowles et al., Organic Letters 2006, 8, 1161-1163. The method of Scheme 13 is illustrated in Step A of Example 7.

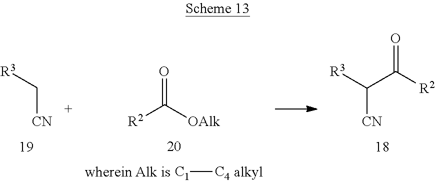

Compounds of Formulae 19 and 20 are commercially available or readily prepared by known methods.

Alternatively compounds of Formula 16 can be converted to compounds of Formula if by treatment with a sulfonating reagent similar to the method of Scheme 3, followed by reduction of the resulting sulfonate with a formate salt or silane such as triethylsilane and a palladium catalyst as illustrated in Scheme 14.

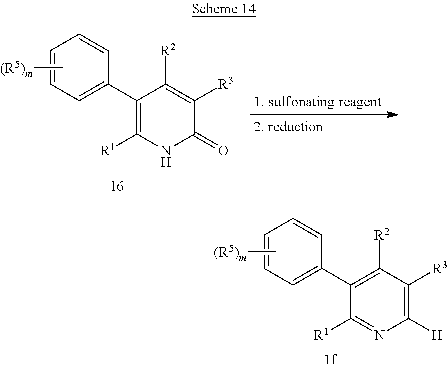

Suitable ligands for the palladium catalyst include triphenylphosphine, 1,1′-bis(diphenylphosphino)ferrocene and 1,1′-(1,3-propanediyl)bis[1,1-diphenyl]phosphine. Examples of reactions of this type can be found in Subramanian et al., Synthesis 1984, 6, 481-485; Kotsuki et al., Synthesis 1995, 11, 1348-1350 and Cacchi et al., Tetrahedron Letters 1986, 27, 5541-5554. The method of Scheme 14 is illustrated in Step E of Example 10 and Step E of Example 13.

Besides the method of Scheme 12, compounds of Formula 16 can be prepared by a diazotization and hydrolysis of amines of Formula 21 as shown in Scheme 15.

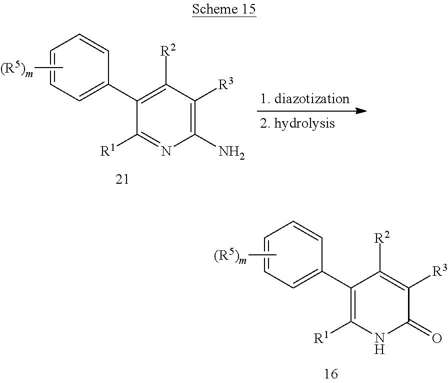

Suitable diazotization reagents include sodium nitrite and alkyl nitrites. Suitable solvents include aqueous hydrochloric or sulfuric acid and aqueous acetic acid. The reactions are typically run at temperatures ranging from 0 to 100° C. Examples of reactions of this type can be found in Carroll et al., Journal of Medicinal Chemistry 2001, 44, 2229-2237 and Smith et al., Organic Syntheses 2002, 78, 51-62. The method of Scheme 15 is illustrated in Step D of Example 9 and Step D of Example 13.

Amines of Formula 21 can be prepared by oxidation of dihydropyridines of Formula 22 as shown in Scheme 16.

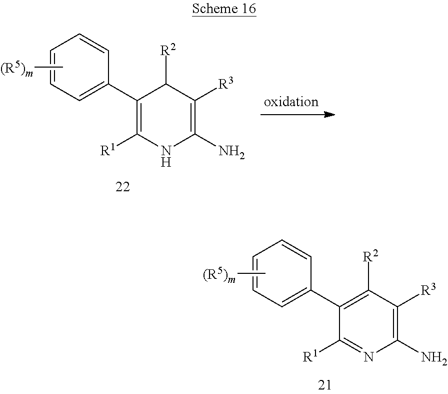

Suitable oxidizing reagents include manganese(IV) oxide and 4,5-dichloro-3,6-dioxo-1,4-cyclohexadiene-1,2-dicarbonitrile (DDQ). Suitable solvents include dichloromethane, chloroform, N,N-dimethylformamide and acetic acid. The reactions are typically run at temperatures ranging from room temperature to 150° C. Examples of reactions of this type can be found in Evdokimov et al., Journal of Organic Chemistry 2007, 72, 3443-3453 and Guo et al., Tetrahedron 2007, 63, 5300-5311. The method of Scheme 16 is illustrated in Step C of Example 9.

Dihydropyridines of Formula 22 can be prepared by condensation of carbonyl compounds of Formula 23 with amidines of Formula 24 as shown in Scheme 17.

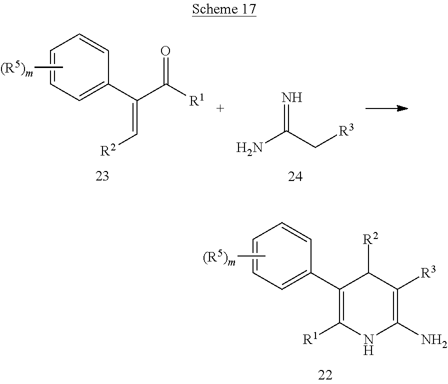

Optionally amine bases such as piperidine or alkali metal alkoxides can be employed in this reaction. Suitable solvents include alcohols such as ethanol and 2-propanol. The reactions are typically run at temperatures ranging from room temperature to 150° C. Examples of reactions of this type can be found in Kobayashi et al., Chemical and Pharmaceutical Bulletin 1995, 43, 788-796 and Meyer et al. Justus Liebigs Annalen der Chemie 1977, 11-12, 1895-1908. The method of Scheme 17 is illustrated in Step B of Example 9.

Enones of Formula 23 can be prepared by a Knoevenagel-type condensation of an aldehyde of Formula 25 with a ketone of Formula 17 as shown in Scheme 18.

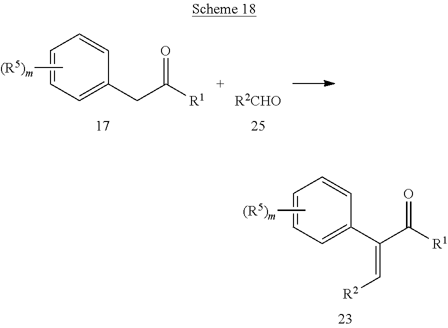

Suitable catalysts for this reaction included amine bases such as piperidine, or reagents such as acetic acid or sodium acetate. For a review article on condensations of this type see G. Jones in Organic Reactions, vol. 15, pp 204-599, A. C. Cope, Ed., John Wiley, New York (1967).

Amidines of Formula 24 are either commercially available or readily prepared by known methods.

Particularly useful intermediates for the preparation of compounds of Formula 1 are compounds of Formula 26 (wherein X² is a leaving group such as Br or I). As illustrated in Scheme 19, these intermediates can be converted to compounds of Formula 1 by methods similar to those described for Scheme 2.

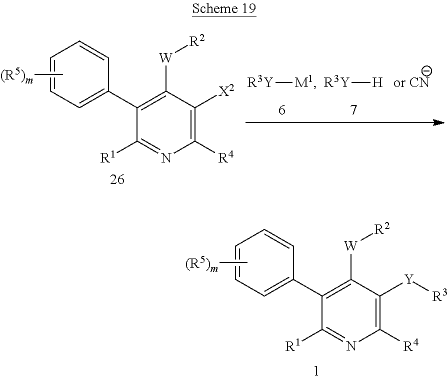

The method of Scheme 19 is illustrated in Example 14.

As illustrated in Scheme 20, compounds of Formula 26 can be prepared from hydrogen compounds of Formula 27 when R⁴ is an electron donating group such as an amine or hydroxyl group by methods similar to those described for Scheme 4.

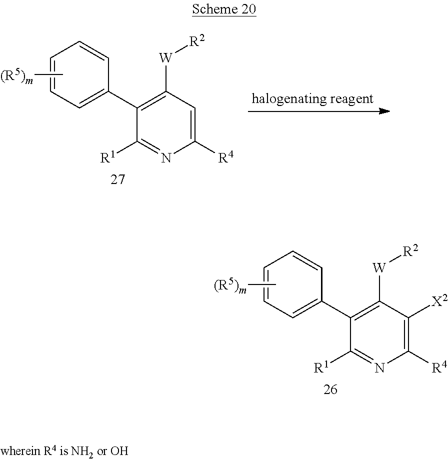

The method of Scheme 20 is illustrated in Step C of Example 13.

Compounds of Formula 27 can be prepared by from esters of Formula 14 by saponfication and decarboxylation as illustrated in Scheme 21.

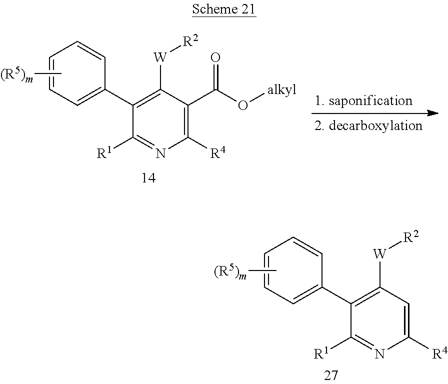

Saponfication reactions are well-known to one skilled in the art. Decarboxylation reactions are generally conducted thermally at temperatures ranging from 50 to 300° C. The reactions can be performed neat or in solvents such as Dowtherm® A or quinoline. Suitable catalysts include copper. Examples of this reaction type can be found in Church et al., Journal of Organic Chemistry 1995, 60, 3750-3758 and PCT Publication WO 2005/1003537. The method of Scheme 21 is illustrated in Step B of Example 13.

One skilled in the art recognizes that the above Schemes are illustrative of a wide variety of general methodologies suitable for preparing compounds of Formula 1, and that variations of these methods and extensions beyond the range of substituents particularly described above are useful. Moreover, in some cases it may be more convenient to perform a combination of the steps illustrated in the above Schemes in an order other than that implied by the particular sequence presented to prepare compounds of Formula 1. For example, compounds of Formula 2, (useful for preparing compounds of Formula 1) can be prepared using methods analogous to Schemes 2 through 5 wherein the phenyl ring optionally substituted with R⁵ is introduced in the last step and YR³ is present in the first step. This is further illustrated in Steps A through F of Example 5.

Without further elaboration, it is believed that one skilled in the art using the preceding description can utilize the present invention to its fullest extent. The following Examples are, therefore, to be construed as merely illustrative, and not limiting of the disclosure in any way whatsoever. Steps in the following Examples illustrate a procedure for each step in an overall synthetic transformation, and the starting material for each step may not have necessarily been prepared by a particular preparative run whose procedure is described in other Examples or Steps. In the following Examples, the term “degassed” when used in connection with a solvent refers to a solvent in which atmospheric oxygen was removed before use by sparging the solvent with nitrogen. Percentages are by weight except for chromatographic solvent mixtures or where otherwise indicated. Parts and percentages for chromatographic solvent mixtures are by volume unless otherwise indicated. ¹H NMR spectra are reported in ppm downfield from tetramethylsilane; “s” means singlet, “d” means doublet, “t” means triplet, “q” means quartet, “m” means multiplet, “dd” means doublet of doublets, and “br s” means broad singlet.

### Example 1

### Preparation of ethyl 4-(3,5-dimethoxyphenyl)-5-(4-fluorophenyl)-6-methyl-3-pyridinecarboxylate (Compound II)

### Step A: Preparation of 1,3-diethyl-2-[[[1-(4-fluorophenyl)-1-propen-1-yl]amino]-methylene]propanedioate

To a solution of diethyl aminomethylenemalonate (10 g, 53 mmol) and 1-(4-fluorophenyl)-2-propanone (7.21 mL, 54 mmol) in tetrahydrofuran (100 mL) was added phosphorus pentoxide (13.5 g, 95.4 mmol). The reaction mixture was stirred overnight, and then more phosphorus pentoxide (13.5 g, 95.4 mmol) was added. The reaction mixture was stirred overnight again, and then the tetrahydrofuran was decanted from the reaction mixture and more tetrahydrofuran (100 mL) was added. After repeating this process five times the mixture was concentrated under reduced pressure. The resulting residue was diluted with ethyl acetate and water, and the layers were separated. The organic layer was dried over sodium sulfate, filtered and concentrated under reduced pressure. The resulting oil was purified by medium pressure liquid chromatography (0 to 30% ethyl acetate in hexanes as eluant) to provide an isomeric mixture of the title compound, as a tan oil (5.8 g).

### Step B Preparation of ethyl 5-(4-fluorophenyl)-1,4-dihydro-6-methyl-4-oxo-3-pyridinecarboxylate

To Dowtherm® A (biphenyl-diphenyl ether mixture) (30 mL) heated at reflux was added 1,3-diethyl-2-[[[1-(4-fluorophenyl)-1-propen-1-yl]amino]methylene]-propanedioate (i.e. the product of Step A) (5.8 g). After 7 minutes, the reaction mixture was cooled and mixed with silica gel. The silica gel mixture was purified by medium pressure liquid chromatography (starting with 50% toluene in hexanes as eluant to remove the Dowtherm® A, and then 0 to 8% methanol in dichloromethane as eluant) to provide the title compound as a pale yellow solid (2.35 g).

¹H NMR (CDCl₃): δ 11.37 (s, 1H), 8.88 (s, 1H), 7.24 (m, 2H), 7.16 (m, 2H), 4.47 (q, 2H), 2.35, (s, 3H), 1.45 (t, 3H).

### Step C Preparation of ethyl 4-chloro-5-(4-fluorophenyl)-6-methyl-3-pyridinecarboxylate

A mixture of ethyl 5-(4-fluorophenyl)-1,4-dihydro-6-methyl-4-oxo-3-pyridine-carboxylate (i.e. the product of Step B) (1.5 g) and phosphorus oxychloride (10 mL) was heated at reflux for 2 h. The reaction mixture was cooled and concentrated under reduced pressure. Toluene was added to the reaction mixture, and the mixture was concentrated under reduced pressure. The resulting residue was dissolved in dichloromethane and washed with saturated aqueous sodium bicarbonate. The organic layer was dried over sodium sulfate, filtered and concentrated under reduced pressure. The resulting residue was filtered through silica gel with dichloromethane as eluant to provide the title compound as a tan oil (1.5 g).

¹H NMR (CDCl₃): δ 8.86 (s, 1H), 7.17 (m, 4H), 4.44 (q, 2H), 2.35, (s, 3H), 1.42 (t, 3H).

### Step D Preparation of ethyl 4-(3,5-dimethoxyphenyl)-5-(4-fluorophenyl)-6-methyl-3-pyridinecarboxylate

A mixture of ethyl 4-chloro-5-(4-fluorophenyl)-6-methyl-3-pyridinecarboxylate (i.e. the product of Step C) (1.5 g, 5.7 mmol), 2-(3,5-dimethoxyphenyl)-4,4,5,5-tetramethyl-1,3,2-dioxaborolane (2.42 g 9.17 mmol), potassium phosphate (2.43 g, 11.5 mmol), palladium acetate (0.047 g, 0.21 mmol) and 2-dicyclohexylphosphino-2′,6′-dimethoxybiphenyl was heated at 125° C. for 1 h. The reaction mixture was cooled, dichloromethane was added and the layers were separated. The organic layer was washed with water and concentrated under reduced pressure. The resulting residue was purified by medium pressure liquid chromatography (10 to 40% ethyl acetate in hexanes as eluant) to provide the title compound, a compound of the present invention, as a white solid (1.0 g).

¹H NMR (CDCl₃): δ 8.91 (s, 1H), 6.96 (m, 4H), 6.26 (t, 1H), 6.07 (d, 2H), 4.10 (q, 2H), 3.63 (s, 6H), 2.40, (s, 3H), 1.02 (t, 3H).

### Example 2

### Preparation of 1-[4-(3,5-dimethoxyphenyl)-5-(4-fluorophenyl)-6-methyl-3-pyridinyl]ethanone (Compound 12) and 4-(3,5-dimethoxyphenyl)-5-(4-fluorophenyl)-α,α,6-trimethyl-3-pyridinemethanol (Compound 13)

To a mixture of ethyl 4-(3,5-dimethoxyphenyl)-5-(4-fluorophenyl)-6-methyl-3-pyridinecarboxylate (i.e. the product of Step D, Example 1) (0.90 g) in diethyl ether (70 mL) was added a solution of methylmagnesium iodide in ether (3 M, 1.14 mL, 3.42 mmol). The reaction mixture was stirred overnight, then saturated aqueous ammonium chloride was added and the aqueous mixture was extracted with dichloromethane. The organic layer was dried over sodium sulfate, filtered and concentrated under reduced pressure. The resulting residue was purified by medium pressure liquid chromatography (15 to 100% ethyl acetate in hexanes as eluant) to provide 1-[4-(3,5-dimethoxyphenyl)-5-(4-fluorophenyl)-6-methyl-3-pyridinyl]ethanone, a compound of the present invention, as a white solid (0.110 g).

¹H NMR (CDCl₃): δ 8.66 (s, 1H), 6.97 (m, 4H), 6.30 (t, 1H), 6.09 (d, 2H), 3.63 (s, 6H), 2.40, (s, 3H), 1.98 (s, 3H).

Also isolated was methyl 4-(3,5-dimethoxyphenyl)-5-(4-fluorophenyl)-α,α,6-trimethyl-3-pyridinemethanol, a compound of the present invention, as a pale yellow solid (0.414 g).

¹H NMR (CDCl₃): δ 8.79 (s, 1H), 6.88 (m, 4H), 6.21 (t, 1H), 6.15 (d, 2H), 3.66 (s, 6H), 2.27, (s, 3H), 1.54 (s, 6H).

### Example 3

### Preparation of 4-(3,5-dimethoxyphenyl)-3-(4-fluorophenyl)-2-methyl-5-(1-methylethenyl)pyridine (Compound 16)

To 4-(3,5-dimethoxyphenyl)-5-(4-fluorophenyl)-α,α,6-trimethyl-3-pyridinemethanol (i.e. a product of Example 2) (0.200 g, 0.525 mmol) in toluene (10 mL) was added p-toluenesulfonic acid (0.100 g, 0.525 mmol). The reaction mixture was heated at reflux for 5 h, then cooled and saturated aqueous sodium bicarbonate was added. The aqueous mixture was extracted with dichloromethane. The organic layer was dried over sodium sulfate, filtered and concentrated under reduced pressure. The resulting residue was purified by medium pressure liquid chromatography (15 to 80% ethyl acetate in hexanes as eluant) to provide the title compound, a compound of the present invention, as a white solid (0.021 g).

¹H NMR (CDCl₃): δ 8.42 (s, 1H), 6.94 (m, 4H), 6.24 (t, 1H), 6.11 (d, 2H), 5.10 (dd, 1H), 5.03 (m, 1H), 3.62 (s, 6H), 2.35, (s, 3H), 1.58 (s, 3H).

### Example 4

### Preparation of 4-(3,5-dimethoxyphenyl)-3-(4-fluorophenyl)-2-methyl-5-(1-methylethyl)pyridine (Compound 19)

To a mixture of 4-(3,5-dimethoxyphenyl)-3-(4-fluorophenyl)-2-methyl-5-(1-methylethenyl)pyridine (i.e. the product of Example 3) (0.072 g) and 10% palladium on carbon (0.008 g) was added ethanol (10 mL). A balloon filled with hydrogen was connected to the reaction flask, and the reaction mixture was stirred at room temperature for 3 h. After standing for 3 days under a nitrogen atmosphere the catalyst was removed by filtration. The reaction mixture was concentrated under reduced pressure to afford the title compound, a compound of the present invention, as a white solid (0.081 g).

¹H NMR (CDCl₃): δ 8.54 (s, 1H), 6.96 (m, 2H), 6.88 (m, 2H), 6.25 (t, 1H), 6.07 (d, 2H), 3.67 (s, 6H), 2.81 (m, 1H), 2.30 (s, 3H), 1.21 (d, 6H).

### Example 5

### Preparation of 4-(3,5-dimethoxyphenyl)-3-(4-fluorophenyl)-5-phenyl-2-methylpyridine (Compound 34)

### Step A: Preparation of 3-(4-fluorophenyl)-2-methyl-4H-pyran-4-one

A mixture of 2-methyl-4-oxo-4H-pyran-3-yl 1,1,1-trifluoromethanesulfonate (prepared according to Lopez et al., Tetrahedron Letters 2007, 48, 2063-2065) (11.6 g 45 mmol) potassium trifluoro(4-fluorophenyl)borate (10 g, 49.5 mmol), cesium carbonate (44 g, 135 mmol), tricyclohexylphosphine (1.26 g, 4.5 mmol) and palladium acetate (0.505 g, 2.25 mmol) in degassed tetrahydrofuran (150 mL) and degassed water (15 mL) was heated at reflux overnight. The reaction mixture was concentrated under reduced pressure, and then saturated sodium chloride and ethyl acetate were added to the resulting residue. The mixture was filtered through diatomaceous earth, and the organic layer was dried over sodium sulfate, filtered and concentrated under reduced pressure. The resulting residue was crystallized with 1-chlorobutane to form a solid, which was removed by filtration. The filtered and the filtrate was purified by medium pressure liquid chromatography (60 to 100% ethyl acetate in hexanes as eluant) to provide the title compound as a brown oil (1.06 g).

¹H NMR (CDCl₃): δ 7.72 (d, 1H), 7.22 (m, 2H), 7.11 (m, 2H), 6.42 (d, 1H), 2.20 (s, 3H).

### Step B Preparation of 3-(4-fluorophenyl)-2-methyl-4(1H)-pyridinone

A mixture of 3-(4-fluorophenyl)-2-methyl-4H-pyran-4-one (i.e. the product of Step A) (1.06 g, 5.2 mmol), ethanol (10 mL) and concentrated ammonium hydroxide (10 mL) was heated in a sealed vessel overnight at 90° C. After cooling to room temperature the solvent was removed under reduced pressure. The resulting residue was triturated with hot 1-chlorobutane to provide the title compound as a solid (0.81 g).

¹H NMR (DMSO-d₆): δ 11.39 (br s, 1H), 7.54 (br s, 1H), 7.21 (m, 4H), 6.10 (br s, 1H), 2.06 (s, 3H).

### Step C Preparation of 5-bromo-3-(4-fluorophenyl)-2-methyl-4(1H)-pyridinone

To a mixture of 3-(4-fluorophenyl)-2-methyl-4(1H)-pyridinone (i.e. the product of Step B) (0.81 g, 4.0 mmol) in acetic acid (7 mL) was added dropwise a solution of bromine (0.268 mL, 5.2 mmol) in acetic acid (1 mL). The reaction mixture was stirred for 4.5 h and then water was added. The aqueous mixture was filtered, and the collected solid was dried in a vacuum oven to provide the title compound as a tan solid (1.23 g).

¹H NMR (DMSO-d₆): δ 11.98 (br s, 1H), 8.18 (s, 1H), 7.23 (m, 4H), 2.06 (s, 3H).

### Step D Preparation of 5-bromo-4-chloro-3-(4-fluorophenyl)-2-methylpyridine

A mixture of 5-bromo-3-(4-fluorophenyl)-2-methyl-4(1H)-pyridinone (i.e. the product of Step C) (1.23 g, 4.36 mmol) and phosphorus oxychloride (10 mL) was heated at reflux for 1.5 h. After cooling to room temperature the reaction mixture was concentrated under reduced pressure. Saturated aqueous sodium bicarbonate was added to the resulting residue and the mixture was extracted with dichloromethane. The organic layer was dried over sodium sulfate, filtered and concentrated under reduced pressure. The resulting residue was filtered through silica gel with dichloromethane as eluant to provide the title product as a tan oil (0.987 g).

¹H NMR (CDCl₃): δ 8.63 (s, 1H), 7.17 (m, 4H), 2.28 (s, 3H).

### Step E Preparation of 4-chloro-3-(4-fluorophenyl)-5-phenyl-2-methylpyridine

A mixture of 5-bromo-4-chloro-3-(4-fluorophenyl)-2-methylpyridine (i.e. the product of Step D) (0.100 g, 0.33 mmol), benzeneboronic acid (0.045 g, 0.37 mmol), sodium bicarbonate (0.112 g, 1.33 mmol), triphenylphosphine (0.028 g, 0.11 mmol) and tris(dibenzylideneacetone)dipalladium (0.012 g, 0.013 mmol) in degassed 1,2-dimethoxyethane (5 mL) and degassed water (1 mL) was heated at reflux overnight. The reaction mixture was diluted with water and extracted with dichloromethane. The organic layer was dried over sodium sulfate, filtered and concentrated under reduced pressure. The resulting residue was filtered through silica gel with dichloromethane as eluant to provide the title compound as a tan oil (0.116 g).

### Step F Preparation of 4-(3,5-dimethoxyphenyl)-3-(4-fluorophenyl)-5-phenyl-2-methylpyridine

A mixture of 4-chloro-3-(4-fluorophenyl)-5-phenyl-2-methylpyridine (i.e. the product of Step E) (0.116 g, 0.39 mmol), 3,5-dimethoxybenzeneboronic acid (0.114 g, 0.624 mmol), potassium phosphate (0.136 g, 0.63 mmol), palladium acetate (0.06 g, 0.027 mmol) and (2,6-dimethoxy-1,1′-biphenyl-2-yl)dicyclohexylphosphine (0.022 g, 0.54 mmol) in degassed N,N-dimethylformamide (3.5 mL) and water (0.175 mL) was heated at 130° C. for 5 h. The reaction mixture was allowed to stand at room temperature overnight, then water was added and the aqueous mixture was extracted with dichloromethane. The organic layer was dried over sodium sulfate, filtered and concentrated under reduced pressure. The resulting residue was purified by medium pressure liquid chromatography (5 to 30% ethyl acetate in hexanes as eluant) to provide the title compound, a compound of the present invention, as a colorless oil (0.090 g).

¹H NMR (CDCl₃): δ 8.56 (s, 1H), 7.21 (m, 3H), 7.13 (m, 2H), 7.02 (m, 2H), 6.94 (m, 2H), 6.11 (t, 1H), 5.91 (d, 2H), 3.47 (s, 6H), 2.41, (s, 3H).

### Example 6

### Preparation of 4-(3,5-dimethoxyphenyl)-5-(2-fluorophenyl)-3-(4-fluorophenyl)-2-methylpyridine 1-oxide (Compound 39)

To a solution of 4-(3,5-dimethoxyphenyl)-5-(2-fluorophenyl)-3-(4-fluorophenyl)-2-methylpyridine (prepared by a method analogous to Example 5) (0.100 g, 0.24 mmol) in dichloromethane (5 mL) at 0° C. was added 3-chloroperoxybenzoic acid (70%, 0.059 g, 0.24 mmol). The reaction mixture was stirred overnight at room temperature, and then more 3-chloroperoxybenzoic acid (70%, 0.033 g, 0.0.13 mmol) was added. The reaction mixture was stirred overnight again, then methyl sulfide (2 drops) was added and stirring was continued for 20 minutes. The reaction mixture was washed with saturated aqueous sodium bicarbonate, and the organic layer was dried over sodium sulfate, filtered and concentrated under reduced pressure. The resulting residue was purified by medium pressure liquid chromatography (5 to 30% ethyl acetate in hexanes as eluant) to provide the title compound, a compound of the present invention, as a tan solid (0.067 g).

¹H NMR (CDCl₃): δ 8.37 (s, 1H), 7.03 (m, 4H), 6.97 (m, 4H), 6.09 (t, 1H), 5.89 (d, 2H), 3.48 (s, 6H), 2.41, (s, 3H).

### Example 7

### Preparation of 2-chloro-3-cyclopentyl-4-(3,5-dimethoxyphenyl)-5-(4-fluorophenyl)-6-methylpyridine (Compound 49)

### Step A: Preparation of α-cyclopentyl-3,5-dimethoxy-β-oxobenzenepropanenitrile

To a solution of cyclopentaneacetonitrile (2.18 g, 20 mmol) in tetrahydrofuran (30 mL) was added dropwise a solution of potassium tent-pentoxide in toluene (1.7 M, 35 mL, 60 mmol), followed by dropwise addition of a solution of methyl 3,5-dimethoxybenzoate (5.88, 30 mmol) in tetrahydrofuran (30 mL). The reaction mixture was stirred overnight, then poured into 1 N HCl, and ethyl acetate was added to the aqueous mixture. The layers were separated, and the organic layer was dried over sodium sulfate, filtered and concentrated under reduced pressure. The resulting residue was purified by medium pressure liquid chromatography (5 to 30% ethyl acetate in hexanes as eluant) to provide the title compound as a white solid (3.63 g).

¹H NMR (CDCl₃): δ 7.06 (d, 2H), 6.71 (t, 1H), 4.32 (d, 2H), 3.85 (s, 6H), 2.53 (m, 1H), 1.88 (m, 2H), 1.75 (m, 2H), 1.58 (m, 3H), 1.40 (m, 1H).

### Step B Preparation of 3-cyclopentyl-4-(3,5-dimethoxyphenyl)-5-(4-fluorophenyl)-6-methyl-2(1H)-pyridinone

To a mixture of α-cyclopentyl-3,5-dimethoxy-β-oxobenzenepropanenitrile (i.e. the product of Step A) (1.09 g, 4.0 mmol) and 1-(4-fluorophenyl)-2-propanone (1.06 mL, 8.0 mol) was added sulfuric acid (0.4 mL). The reaction mixture was stirred and heated at 100 to 110° C. overnight. After cooling to room temperature, 1 N NaOH and 1-chlorobutane were added to the reaction mixture, the mixture was filtered, and the filtrate was concentrated under reduced pressure. The resulting residue was purified by medium pressure liquid chromatography (5 to 20% acetone in chloroform as eluant) to provide the title compound (0.21 g). An additional quantity (0.21 g) of title compound mixed with some impurities was also isolated.

¹H NMR (CDCl₃): δ 6.85 (m, 4H), 6.22 (t, 1H), 6.04 (d, 2H), 3.66 (s, 6H), 2.70 (m, 1H), 2.18 (m, 2H), 2.11, (s, 3H), 1.84 (m, 2H), 1.55 (m, 4H).

### Step C Preparation of 2-chloro-3-cyclopentyl-4-(3,5-dimethoxyphenyl)-5-(4-fluorophenyl)-6-methylpyridine

A mixture of 3-cyclopentyl-4-(3,5-dimethoxyphenyl)-5-(4-fluorophenyl)-6-methyl-2(1H)-pyridinone (i.e. the product of Step B) (0.18 g, 0.44 mmol) and phenylphosphonic dichloride (1 mL) was heated at 170° C. for 4 h. After cooling, the reaction mixture was poured into a mixture of ice/concentrated ammonium hydroxide and extracted with dichloromethane. The organic layer was dried over sodium sulfate, filtered and concentrated under reduced pressure. The resulting residue was purified by medium pressure liquid chromatography (5 to 70% ethyl acetate in hexanes as eluant) to afford the title compound, a compound of the present invention, as a white solid (0.071 g).

¹H NMR (CDCl₃): δ 6.90 (m, 4H), 6.24 (t, 1H), 6.04 (d, 2H), 3.66 (s, 6H), 2.99 (m, 1H), 2.26, (s, 3H), 2.18 (m, 2H), 1.87 (m, 2H), 1.68 (m, 2H) 1.53 (m, 2H).

### Example 8

### Preparation of 5-cyclopentyl-4-(3,5-dimethoxyphenyl)-3-(4-fluorophenyl)-2-methylpyridine (Compound 50)

To a mixture of 2-chloro-3-cyclopentyl-4-(3,5-dimethoxyphenyl)-5-(4-fluorophenyl)-6-methylpyridine (i.e. the product of Example 7) (0.050 g) and 10% palladium on carbon (0.025 g) was added ethanol (5 mL) and triethylamine (0.1 mL). A balloon filled with hydrogen was connected to the reaction flask, and the reaction mixture was stirred at room temperature overnight, then filtered, and the filtrate was concentrated under reduced pressure. The resulting residue was filtered through silica gel with 15% ethyl acetate in hexanes as eluant to provide the title product, a compound of the present invention, as a white solid (0.041 g) melting at 129-130° C.

¹H NMR (CDCl₃) δ 8.54 (s, 1H), 6.96 (m, 2H), 6.88 (m, 2H), 6.24 (t, 1H), 6.07 (d, 2H), 3.66 (s, 6H), 2.80 (m, 1H), 2.30 (s, 3H), 1.90 (m, 2H), 1.80 (m, 2H), 1.64 (m, 2H) 1.55 (m, 2H).

### Example 9

### Preparation of ethyl 4-(3,5-dimethoxyphenyl)-6-methyl-5-(2,4,6-trifluorophenyl)-3-pyridinecarboxylate (Compound 130)

### Step A: Preparation of 4-(3,5-dimethoxyphenyl)-3-(2,4,6-trifluorophenyl)-3-buten-2-one

To a solution of 1-(2,4,6-trifluorophenyl)-2-propanone (prepared according to PCT Publication WO 2006/1175) (14.3 g, 76 mmol) and 3,5-dimethoxybenzaldehyde (12.6 g, 76 mmol) in toluene was added piperidine (2 mL) and acetic acid (0.5 mL). The solution was refluxed overnight under a Dean-Stark condenser. After cooling, the reaction mixture was washed with water. The aqueous layer was extracted with ethyl acetate. The combined organic layers were dried over sodium sulfate, filtered and concentrated under reduced pressure. The residue was purified by medium pressure liquid chromatography (0 to 20% acetone in chloroform as eluant) to provide the title compound (11.4 g).

¹H NMR (CDCl₃): δ 7.80 (s, 1H), 6.73 (dd, 2H), 6.42 (t, 1H), 6.30 (d, 2H), 3.64, (s, 6H), 2.46 (s, 3H).

### Step B Preparation of ethyl 2-amino-4-(3,5-dimethoxyphenyl)-1,4-dihydro-6-methyl-5-(2,4,6-trifluorophenyl)-3-pyridinecarboxylate

To a mixture of 4-(3,5-dimethoxyphenyl)-3-(2,4,6-trifluorophenyl)-3-buten-2-one (i.e. the product of Step A) (17.57 g, 52.3 mmol) and ethyl 3-amino-3-iminopropanoate hydrochloride (9.55 g, 57.5 mmol) in ethanol (350 mL) was added piperidine (6.2 mL, 63 mmol). The reaction mixture was heated at reflux overnight. After cooling to room temperature, the solvent was removed under reduced pressure. The residue was purified by medium pressure liquid chromatography (2 to 20% acetone in chloroform as eluant) to provide the title compound as a light tan solid (16.05 g), found by ¹H NMR to be a mixture of tautomers.

¹H NMR (CDCl₃): major tautomer δ 6.57 (m, 2H), 6.26 (t, 1H), 6.20 (d, 2H) 6.02 (br m, 2H), 5.41 (br s, 1H), 4.46 (br s, 1H), 4.00 (q, 2H), 3.67 (s, 6H), 2.18 (s, 3H), 1.11 (t, 3H).

### Step C Preparation of ethyl 2-amino-4-(3,5-dimethoxyphenyl)-6-methyl-5-(2,4,6-trifluorophenyl)-3-pyridinecarboxylate

A mixture of ethyl 2-amino-4-(3,5-dimethoxyphenyl)-1,4-dihydro-6-methyl-5-(2,4,6-trifluorophenyl)-3-pyridinecarboxylate (i.e. the product of Step B) (16.05 g, 35.8 mmol) and activated manganese(IV) oxide (6.22 g, 71.6 mmol) in dichloromethane (500 mL) was heated at reflux overnight. Additional activated manganese(IV) oxide (6.22 g, 71.6 mmol) was added, and the reaction mixture was heated at reflux for 3 h. A third portion of activated manganese(IV) oxide (6.22 g, 71.6 mmol) was added, and the reaction mixture was heated at reflux for 3 h. After cooling, the reaction mixture was filtered through diatomaceous earth (using tetrahydrofuran for rinsing). The solvent was removed under reduced pressure to provide the title compound as a light tan solid (14 g).

¹H NMR (CDCl₃): δ 6.53 (dd, 2H), 6.26 (t, 1H), 6.19 (d, 2H), 6.08 (br m, 2H), 3.90 (q, 2H), 3.66, (s, 6H), 2.18 (s, 3H), 0.76 (t, 3H).

### Step D Preparation of ethyl 4-(3,5-dimethoxyphenyl)-6-methyl-2-oxo-5-(2,4,6-trifluorophenyl)-3-pyridinecarboxylate

To ethyl 2-amino-4-(3,5-dimethoxyphenyl)-6-methyl-5-(2,4,6-trifluorophenyl)-3-pyridinecarboxylate (i.e. the product of Step C) (8.29 g, 18.6 mmol) in acetic acid (70 mL) at 10° C. was added dropwise a solution of sodium nitrite (5.1 g, 74 mmol) in water (40 mL). The reaction mixture was stirred at room temperature for 4 h and then heated at 50° C. overnight. After cooling, water was added and a solid was isolated by filtration. The tan solid was dried in a vacuum oven to provide the title compound (8.35 g).

¹H NMR (CDCl₃): δ 6.56 (dd, 2H), 6.27 (t, 1H), 6.25 (d, 2H), 4.07 (q, 2H), 3.66 (s, 6H), 2.19 (s, 3H), 0.97 (t, 3H).

### Step E Preparation of ethyl 4-(3,5-dimethoxyphenyl)-6-methyl-5-(2,4,6-trifluorophenyl)-3-pyridinecarboxylate

To a solution of ethyl 4-(3,5-dimethoxyphenyl)-6-methyl-2-oxo-5-(2,4,6-trifluorophenyl)-3-pyridinecarboxylate (i.e. the product of Step D) (8.35 g, 18.6 mmol) in dichloromethane (350 mL) at 0° C. was added triethylamine (5.22 mL, 37.4 mmol) and trifluoromethanesulfonic anhydride (4.74 mL, 29 mmol). The reaction mixture was stirred 1 h at 0° C. The solvent was removed under reduced pressure. Degassed N,N-dimethylformamide (200 mL) was added, followed by triethylamine (26.1 mL, 187 mmol), 1,1′-bis(diphenylphosphino)ferrocene (0.837 g, 1.5 mmol), palladium(II) acetate (0.335 g, 1.5 mmol) and 98% formic acid (3.53 mL, 93.5 mmol). The reaction mixture was heated at 60° C. for 4 h. The reaction mixture was allowed to stand at room temperature overnight. The solvent was removed under reduced pressure. The residue was taken up in ethyl acetate and washed with water. The organic layer was dried over sodium sulfate, filtered and concentrated under reduced pressure. The residue was purified by medium pressure liquid chromatography (15 to 100% ethyl acetate in hexanes as eluant) to provide the title compound, a compound of the present invention, as a white solid (6.68 g). Starting material (0.48 g) was also recovered.

¹H NMR (CDCl₃): δ 8.76 (s, 1H), 6.61 (dd, 2H), 6.35 (t, 1H), 6.22 (d, 2H), 4.11 (q, 2H), 3.68 (s, 6H), 2.41 (s, 3H), 1.03 (t, 3H).

### Example 10

### Preparation of 1-[4-(3,5-dimethoxyphenyl)-6-methyl-5-(2,4,6-trifluorophenyl)-3-pyridinyl]ethanone (Compound 40) and 4-(3,5-dimethoxyphenyl)-α,α,6-trimethyl-5-(2,4,6-trifluorophenyl)-3-pyridinemethanol (Compound 44)

To a solution of methyl lithium (38.8 mL of a 1.6 M solution in ether, 62 mmol) in tetrahydrofuran (50 mL) at −78° C. was added methylmagnesium chloride (12.4 mL of a 3 M solution in tetrahydrofuran, 37.2 mmol). After 1 h at this temperature a solution of ethyl 4-(3,5-dimethoxyphenyl)-6-methyl-5-(2,4,6-trifluorophenyl)-3-pyridine-carboxylate (i.e. the product of Example 9) (6.68 g, 15.5 mmol) in tetrahydrofuran (50 mL) at −78° C. was added via cannula. The reaction mixture was stirred 8 h at −70° C. A saturated aqueous solution of ammonium chloride was added. The reaction mixture was extracted with ethyl acetate. The organic layer was dried over sodium sulfate, filtered and concentrated under reduced pressure. The residue was purified by medium pressure liquid chromatography (15 to 100% ethyl acetate in hexanes as eluant) to provide the title compounds, compounds of the present invention, as a white solids (1.07 g of Compound 40 and 4.29 g of Compound 44). Compound 40

¹H NMR (CDCl₃): δ 8.76 (s, 1H), 6.59 (dd, 2H), 6.31 (t, 1H), 6.20 (d, 2H), 3.67 (s, 6H), 2.41 (s, 3H), 2.02 (s, 3H).

### Compound 44

¹H NMR (CDCl₃): δ 8.88 (s, 1H), 6.52 (dd, 2H), 6.27 (m, 3H), 3.69 (s, 6H), 2.31 (s, 3H), 1.53 (s, 6H).

### Example 11

### Preparation of 4-(3,5-dimethoxyphenyl)-6-methyl-5-(2,4,6-trifluorophenyl)-3-pyridinemethanol (Compound 96)

To a solution of lithium aluminum hydride (0.053 g, 1.4 mmol) in tetrahydrofuran (10 mL) at 0° C. was added a solution of ethyl 4-(3,5-dimethoxyphenyl)-6-methyl-5-(2,4,6-trifluorophenyl)-3-pyridinecarboxylate (i.e. the product of Example 9) (0.60 g, 1.4 mmol) in tetrahydrofuran (10 mL). The reaction mixture was stirred at 0° C. for 40 min. Water (0.053 mL), 15% aqueous sodium hydroxide (0.053 mL) and water (0.159 mL) were added sequentially. After 20 min the reaction mixture was filtered through diatomaceous earth. The solvent was removed under reduced pressure to provide the title compound, a compound of the present invention, as a pale yellow oil (0.514 g).

¹H NMR (CDCl₃): δ 8.71 (s, 1H), 6.57 (dd, 2H), 6.32 (t, 1H), 6.23 (d, 2H), 4.52 (d, 2H), 3.70 (s, 6H), 2.37 (s, 3H).

### Example 12

### Preparation of 4-(3,5-dimethoxyphenyl)-6-methyl-5-(2,4,6-trifluorophenyl)-3-pyridineacetonitrile (Compound 124)

To a solution of 4-(3,5-dimethoxyphenyl)-6-methyl-5-(2,4,6-trifluorophenyl)-3-pyridinemethanol (i.e. the product of Example 11) (0.38 g, 0.98 mmol) in dichloromethane (15 mL) at 0° C. was added methanesulfonyl chloride (0.084 mL, 1.1 mmol) and triethylamine (0.18 mL, 1.3 mmol). The reaction mixture was stirred at room temperature for 1 h. The organic layer was washed with water. The water layer was extracted with dichloromethane. The combined organic layers were dried over sodium sulfate, filtered and concentrated under reduced pressure. N,N-dimethylformamide (7.6 mL) was added to the crude mesylate. A 3.6 mL portion of this solution (0.46 mmol) was added to potassium cyanide (0.033 g, 0.51 mmol) in N,N-dimethylformamide (2 mL). An additional quantity of N,N-dimethylformamide (3.6 mL) was added. The reaction mixture was stirred overnight. Dichloromethane was added to the reaction mixture. The organic phase was washed with water. The water layer was extracted with dichloromethane. The combined organic phases were dried over sodium sulfate, filtered and concentrated under reduced pressure. The residue was purified by medium pressure liquid chromatography (15 to 40% ethyl acetate in hexanes as eluant) to provide the title compound, a compound of the present invention, as a white solid (0.121 g).

¹H NMR (CDCl₃): δ 8.72 (s, 1H), 6.59 (dd, 2H), 6.35 (t, 1H), 6.20 (d, 2H), 3.71 (s, 6H), 3.51 (s, 2H), 2.36 (s, 3H).

### Example 13

### Preparation of 5-bromo-4-(3,5-dimethoxyphenyl)-2-methyl-3-(2,4,6-trifluorophenyl)pyridine (Compound 122)

### Step A: Preparation of 2-amino-4-(3,5-dimethoxyphenyl)-6-methyl-5-(2,4,6-trifluorophenyl)-3-pyridinecarboxylic acid

To a solution of ethyl 2-amino-4-(3,5-dimethoxyphenyl)-6-methyl-5-(2,4,6-trifluorophenyl)-3-pyridinecarboxylate (i.e. the product of Step C of Example 9) (7.0 g, 15.7 mmol) in ethanol (200 mL) was added sodium hydroxide (39 mL of a 1 N aqueous solution, 39 mmol). The reaction mixture was heated at 60° C. overnight. After cooling the ethanol was removed under reduced pressure. Water (400 mL) was added. The aqueous layer was washed with ethyl acetate. The pH was adjusted to 5 with concentrated HCl. The aqueous layer was extracted with dichloromethane. The combined organic phases were dried over sodium sulfate, filtered and concentrated under reduced pressure to provide the title compound, as a white solid (4.84 g).

¹H NMR (CDCl₃): δ 6.49 (dd, 2H), 6.22 (t, 1H), 6.12 (d, 2H), 3.60 (s, 6H), 2.04 (s, 3H).

### Step B Preparation of 4-(3,5-dimethoxyphenyl)-6-methyl-5-(2,4,6-trifluorophenyl)-2-pyridinamine

A solution of 2-amino-4-(3,5-dimethoxyphenyl)-6-methyl-5-(2,4,6-trifluorophenyl)-3-pyridinecarboxylic acid (i.e. the product of Step A) (4.8 g, 11.5 mmol) in quinoline (15 mL) was divided into two equal portions. To each portion was added copper powder (0.015 g) and both reaction mixtures were heated in sealed vials in a microwave at 230° C. for 1 h. After cooling the contents of the vials were combined. The quinoline was removed by bulb to bulb distillation (oven temperature 90° C.@ 1 ton). The residue was purified by medium pressure liquid chromatography (20 to 65% ethyl acetate in hexanes as eluant) to provide the title compound, as a tan solid (3.78 g).

¹H NMR (CDCl₃): δ 6.59 (dd, 2H), 6.42 (s, 1H), 6.32 (t, 1H), 6.25 (d, 2H), 4.54 (br, 2H), 3.67 (s, 6H), 2.22 (s, 3H).

### Step C Preparation of 3-bromo-4-(3,5-dimethoxyphenyl)-6-methyl-5-(2,4,6-trifluorophenyl)-2-pyridinamine

To a solution of 4-(3,5-dimethoxyphenyl)-6-methyl-5-(2,4,6-trifluorophenyl)-2-pyridinamine (i.e. the product of Step B) (3.78 g, 7.86 mmol) in dichloromethane (80 mL) at 0° C. was added N-bromosuccinimide (1.5 g, 8.43 mmol) The reaction mixture was stirred at 0° C. for 2 h then room temperature for 1 h. An additional quantity (0.24 g, 1.35 mmol) of N-bromosuccinimide was added. After 25 min the solvent was concentrated under reduced pressure. The residue was purified by medium pressure liquid chromatography (15 to 40% ethyl acetate in hexanes as eluant) to provide the title compound as a white solid (4.08 g).

¹H NMR (CDCl₃): δ 6.53 (dd, 2H), 6.31 (t, 1H), 6.22 (d, 2H), 5.10 (br, 2H), 3.70 (s, 6H), 2.15 (s, 3H).

### Step D Preparation of 3-bromo-4-(3,5-dimethoxyphenyl)-6-methyl-5-(2,4,6-trifluorophenyl)-2(1H)-pyridinone

To 3-bromo-4-(3,5-dimethoxyphenyl)-6-methyl-5-(2,4,6-trifluorophenyl)-2-pyridinamine (i.e. the product of Step C) (4.08 g, 9.15 mmol) in acetic acid (38 mL) at 10° C. was added dropwise a solution of sodium nitrite (2.52 g, 36.6 mmol) in water (22 mL). The reaction mixture was stirred at room temperature for 2 h and then heated at 50° C. overnight. After cooling, water was added and a solid was isolated by filtration. The tan solid was dried in a vacuum oven provide the title compound (4.03 g).

¹H NMR (CDCl₃): δ 6.55 (dd, 2H), 6.31 (t, 1H), 6.22 (d, 2H), 3.71 (s, 6H), 2.21 (s, 3H).

### Step E Preparation of 5-bromo-4-(3,5-dimethoxyphenyl)-2-methyl-3-(2,4,6-trifluorophenyl)pyridine

To 3-bromo-4-(3,5-dimethoxyphenyl)-6-methyl-5-(2,4,6-trifluorophenyl)-2(1H)-pyridinone (i.e. the product of Step D) (4.03 g, 8.87 mmol) in dichloromethane (110 mL) at 0° C. was added triethylamine (2.47 mL, 17.8 mmol) and trifluoromethanesulfonic anhydride (2.25 mL, 13.7 mmol). The reaction mixture was stirred 1 h at room temperature. The solvent was removed under reduced pressure. Degassed N,N-dimethylformamide (100 mL) was added to the residue, followed by triethylamine (12.4 mL, 88.7 mmol), 1,1′-bis(diphenylphosphino)ferrocene (0.32 g, 0.57 mmol), palladium(II) acetate (0.128 g, 0.57 mmol) and 98% formic acid (1.7 mL, 44 mmol). The reaction mixture was heated at 60° C. for 105 min. The reaction mixture was taken up in ether and washed with brine. The aqueous layer was extracted with dichloromethane. The combined organic layers were dried over sodium sulfate, filtered and concentrated under reduced pressure. The residue was purified by medium pressure liquid chromatography (15 to 100% ethyl acetate in hexanes as eluant) to provide the title compound, a compound of the present invention, as a white solid (0.992 g). Starting material (2.51 g) was also recovered.

¹H NMR (CDCl₃): δ 8.77 (s, 1H), 6.58 (dd, 2H), 6.34 (t, 1H), 6.22 (d, 2H), 3.71 (s, 6H), 2.32 (s, 3H).

### Example 14

### Preparation of 4-(3,5-dimethoxyphenyl)-5-(2-fluorophenyl)-2-methyl-3-(2,4,6-trifluorophenyl)pyridine (Compound 89)

To a solution of 5-bromo-4-(3,5-dimethoxyphenyl)-2-methyl-3-(2,4,6-trifluorophenyl)pyridine (i.e. the product of Example 13) (0.090 g, 0.21 mmol) in degassed N,N-dimethylformamide (2.5 mL) was added 2-fluorobenzeneboronic acid (0.038 g, 0.21 mmol), powdered tribasic potassium phosphate (0.090 g, 0.41 mmol), dicyclohexyl(2′,6′-dimethoxy[1,1′-biphenyl]-2-yl)phosphine (0.044 g, 0.11 mmol) and palladium(II) acetate (0.012 g, 0.057 mmol). The reaction mixture was heated at 95° C. overnight. The solvent was removed under reduced pressure. The residue was purified by medium pressure liquid chromatography (5 to 30% ethyl acetate in hexanes as eluant) to provide the title compound, a compound of the present invention, as a white solid (0.101 g).

¹H NMR (CDCl₃): δ 8.59 (s, 1H), 7.22 (m, 1H), 7.09 (m, 1H), 7.02 (m, 2H), 6.60 (dd, 2H), 6.14 (t, 1H), 6.05 (d, 2H), 3.54 (s, 6H), 2.43 (s, 3H).

By the procedures described herein together with methods known in the art, the following compounds of Tables 1 to 4 can be prepared. The following abbreviations are used in the Tables which follow: s means secondary, n means normal, i means iso, c means cyclo, Me means methyl, Et means ethyl, Pr means propyl, i-Pr means isopropyl, Bu means butyl and Ph means phenyl. In the following tables a dash (“-”) in the R⁵ column indicates m is 0 and hydrogen is present at all available positions.

TABLE 1

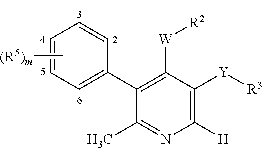

R³

R² is 3,5-di-OMe-Ph; m is 0; W and Y are
both a direct bond.
CO₂Me
CO₂Et
CO₂-n-Pr
C(═O)Me
C(═O)Et
Me
Et
i-Pr
c-Pr
s-Bu
c-pentyl
c-hexyl
C(═CH₂)Me
C(═CH₂)Et
CH₂Cl
CH₂CN
CH═CH₂
C≡CH
Cl
C(═CHCH₃)Me
Ph
2-F—Ph
2-Cl—Ph
3-F—Ph
4-F—Ph
4-Cl—Ph
4-OMe—Ph
4-CF₃—Ph
4-CN—Ph
5-OMe-2-pyridinyl
5-Cl-2-pyridinyl
6-OMe-3-pyridinyl
5-Cl-1,2,4-oxadiazol-3-yl
2-pyridinyl
CH(OH)CH₃
CH(OH)CH₂CH₃
CH(OH)CH(CH₃)₂
C(OH)CH₃)₃
R² is 3,5-di-OMe—Ph; (R⁵)m is 2-F; W and Y
are both a direct bond.
CO₂Me
CO₂Et
CO₂-n-Pr
C(═O)Me
C(═O)Et
Me
Et
i-Pr
c-Pr
s-Bu
c-pentyl
c-hexyl
C(═CH₂)Me
C(═CH₂)Et
CH₂Cl
CH₂CN
CH═CH₂
C≡CH
Cl
C(═CHCH₃)Me
Ph
2-F—Ph
2-Cl—Ph
3-F—Ph
4-F—Ph
4-Cl—Ph
4-OMe—Ph
4-CF₃—Ph
4-CN—Ph
5-OMe-2-pyridinyl
5-Cl-2-pyridinyl
6-OMe-3-pyridinyl
5-Cl-1,2,4-oxadiazol-3-yl
2-pyridinyl
CH(OH)CH₃
CH(OH)CH₂CH₃
CH(OH)CH(CH₃)₂
C(OH)CH₃)₃

R2 is 3,5-di-OMe—Ph; (R5)m is 4-F; W and Y
are both a direct bond.

CO₂Me
CO₂Et
CO₂-n-Pr
C(═O)Me
C(═O)Et
Me
Et
i-Pr
c-Pr
s-Bu
c-pentyl
c-hexyl
C(═CH₂)Me
C(═CH₂)Et
CH₂Cl
CH₂CN
CH═CH₂
C≡CH
Cl
C(═CHCH₃)Me
Ph
2-F—Ph
2-Cl—Ph
3-F—Ph
4-F—Ph
4-Cl—Ph
4-OMe—Ph
4-CF₃—Ph
4-CN—Ph
5-OMe-2-pyridinyl
5-Cl-2-pyridinyl
6-OMe-3-pyridinyl
5-Cl-1,2,4-oxadiazol-3-yl
2-pyridinyl
CH(OH)CH₃
CH(OH)CH₂CH₃
CH(OH)CH(CH₃)₂
C(OH)CH₃)₃
R² is 3,5-di-OMe—Ph; (R⁵)ₘ is 2,4-di-F; W
and Y are both a direct bond.
CO₂Me
CO₂Et
CO₂-n-Pr
C(═O)Me
C(═)Et
Me
Et
i-Pr
c-Pr
s-Bu
c-pentyl
c-hexyl
C(═(CH₂)Me
C(═CH₂)Et
CH₂Cl
CH₂CN
CH═CH₂
C≡CH
Cl
C(═CH(CH₃)Me
Ph
2-F—Ph
2-Cl—Ph
3-F—Ph
4-F—Ph
4-Cl—Ph
4-OMe—Ph
4-CF₃—Ph
4-CN—Ph
5-OMe-2-pyridinyl
5-Cl-2-pyridinyl
6-OMe-3-pyridinyl
5-Cl-1,2,4-oxadiazol-3-yl
2-pyridinyl
CH(OH)CH₃
CH(OH)CH₂CH₃
CH(OH)CH(CH₃)₂
C(OH)CH₃)₃
R² is 3,5-di-OMe—Ph; (R⁵)ₘ is 2,6-di-F; W
and Y are both a direct bond.
CO₂Me
CO₂Et
CO₂-n-Pr
C(═O)Me
C(═OEt
Me
Et
i-Pr
c-Pr
s-Bu
c-pentyl
c-hexyl
C(═CH₂)Me
C(═CH₂)Et
CH₂Cl
CH₂CN
CH═CH₂
C≡CH
Cl
C(═CHCH₃)Me
Ph
2-F—Ph
2-Cl—Ph
3-F—Ph
4-F—Ph
4-Cl—Ph
4-OMe—Ph
4-CF₃—Ph
4-CN—Ph
5-OMe-2-pyridinyl
5-Cl-2-pyridinyl
6-OMe-3-pyridinyl
5-Cl-1,2,4-oxadiazol-3-yl
2-pyridinyl
CH(OH)CH₃
CH(OH)CH₂CH₃
CH(OH)CH(CH₃)₂
C(OH)(CH₃)₃
R² is 3,5-di-OMe—Ph; (R⁵)ₘ is 2,4,6-tri-F; W
and Y are both a direct bond.
CO₂Me
CO₂Et
CO₂-n-Pr
C(═O)Me
C(═O)Et
Me
Et
i-Pr
c-Pr
s-Bu
c-pentyl
c-hexyl
C(═CH₂)Me
C(═CH₂)Et
CH₂Cl
CH₂CN
CH═CH₂
C≡CH
Cl
C(═CHCH₃)Me
Ph
2-F—Ph
2-Cl—Ph
3-F—Ph
4-F—Ph
4-Cl—Ph
4-OMe—Ph
4-CF₃—Ph
4-CN—Ph
5-OMe-2-pyridinyl
5-Cl-2-pyridinyl
6-OMe-3-pyridinyl
5-Cl-1,2,4-oxadiazol-3-yl
2-pyridinyl
CH(OH)CH₃
CH(OH)CH₂CH₃
CH(OH)CH(CH₃)₂
C(OH)(CH₃)₃
R² is 3,5-di-OMe—Ph; (R⁵)ₘ is 4-Cl; W and Y
are both a direct bond.
CO₂Me
CO₂Et
CO₂-n-Pr
C(═O)Me
C(═O)Et
Me
Et
i-Pr
c-Pr
s-Bu
c-pentyl
c-hexyl
C(═CH₂)Me
C(═CH₂)Et
CH₂Cl
CH₂CN
CH═CH₂
C≡CH
Cl
C(═CHCH₃)Me
Ph
2-F—Ph
2-Cl—Ph
3-F—Ph
4-F—Ph
4-Cl—Ph
4-OMe—Ph
4-CF₃—Ph
4-CN—Ph
5-OMe-2-pyridinyl
5-Cl-2-pyridinyl
6-OMe-3-pyridinyl
5-Cl-1,2,4-oxadiazol-3-yl
2-pyridinyl
CH(OH)CH₃
CH(OH)CH₂CH₃
CH(OH)CH(CH₃)₂
C(OH)(CH₃)₃
R² is 3,5-di-OMe—Ph; (R⁵)ₘ is 4-OMe; W and
Y are both a direct bond.
CO₂Me
CO₂Et
CO₂-n-Pr
C(═)Me
C(═O)Et
Me
Et
i-Pr
c-Pr
s-Bu
c-pentyl
c-hexyl
C(═CH₂)Me
C(═CH₂)Et
CH₂Cl
CH₂CN
CH═CH₂
C≡CH
Cl
C(═CHCH₃)Me
Ph
2-F—Ph
2-Cl—Ph
3-F—Ph
4-F—Ph
4-Cl—Ph
4-OMe—Ph
4-CF₃—Ph
4-CN—Ph
5-OMe-2-pyridinyl
5-Cl-2-pyridinyl
6-OMe-3-pyridinyl
5-Cl-1,2,4-oxadiazol-3-yl
2-pyridinyl
CH(OH)CH₃
CH(OH)CH₂CH₃
CH(OH)CH(CH₃)₂
C(OH)(CH₃)₃
R² is 3,5-di-OMe—Ph; (R⁵)ₘ is 2,3,6-tri-F; W
and Y are both a direct bond.
CO₂Me
CO₂Et
CO₂-n-Pr
C(═O)Me
C(═O)Et
Me
Et
i-Pr
c-Pr
s-Bu
c-pentyl
c-hexyl
C(═CH₂)Me
C(═CH₂)Et
CH₂Cl
CH₂CN
CH═CH₂
C≡CH
Cl
C(═CHCH₃)Me
Ph
2-F—Ph
2-Cl—Ph
3-F—Ph
4-F—Ph
4-Cl—Ph
4-OMe—Ph
4-CF₃—Ph
4-CN—Ph
5-OMe-2-pyridinyl
5-Cl-2-pyridinyl
6-OMe-3-pyridinyl
5-Cl-1,2,4-oxadiazol-3-yl
2-pyridinyl
CH(OH)CH₃
CH(OH)CH₂CH₃
CH(OH)CH(CH₃)₂
C(OH)(CH₃)₃
R² is 3,5-di-OMe—Ph; (R⁵)ₘ is 2,6-di-F, 4-
OMe; W and Y are both a direct bond.
CO₂Me
CO₂Et
CO₂-n-Pr
C(═O)Me
C(═O)Et
Me
Et
i-Pr
c-Pr
s-Bu
c-pentyl
c-hexyl
C(═(CH₂)Me
C(═CH₂)Et
CH₂Cl
CH₂CN
CH═CH₂
C≡CH
Cl
C(═CHCH₃)Me
Ph
2-F—Ph
2-Cl—Ph
3-F—Ph
4-F—Ph
4-Cl—Ph
4-OMe—Ph
4-CF₃—Ph
4-CN—Ph
5-OMe-2-pyridinyl
5-Cl-2-pyridinyl
6-OMe-3-pyridinyl
5-Cl-1,2,4-oxadiazol-3-yl
2-pyridinyl
CH(OH)CH₃
CH(OH)CH₂CH₃
CH(OH)CH(CH₃)₂
C(OH)(CH₃)₃
R² is 3,5-di-OMe—Ph; (R⁵)ₘ is 2,6-di-F, 4-
O(CH₂)₃NHCH₃; W and Y are both a direct
bond.
CO₂Me
CO₂Et
CO₂-n-Pr
C(═O)Me
C(═O)Et
Me
Et
i-Pr
c-Pr
s-Bu
c-pentyl
c-hexyl
C(═CH₂)Me
C(═CH₂)Et
CH₂Cl
CH₂CN
CH═CH₂
C≡CH
Cl
C(═CHCH₃)Me
Ph
2-F—Ph
2-Cl—Ph
3-F—Ph
4-F—Ph
4-Cl—Ph
4-OMe—Ph
4-CF₃—Ph
4-CN—Ph
5-OMe-2-pyridinyl
5-Cl-2-pyridinyl
6-OMe-3-pyridinyl
5-Cl-1,2,4-oxadiazol-3-yl
2-pyridinyl
CH(OH)CH₃
CH(OH)CH₂CH₃
CH(OH)CH(CH₃)₂
C(OH)(CH₃)₃
R² is 2-Cl, 3,5-di-OMe—Ph; m is 0; W and Y are
both a direct bond.
CO₂Me
CO₂Et
CO₂-n-Pr
C(═)Me
C(═O)Et
Me
Et
i-Pr
c-Pr
s-Bu
c-pentyl
c-hexyl
C(═CH₂)Me
C(═CH₂)Et
CH₂Cl
CH₂CN
CH═CH₂
C≡CH
Cl
C(═CHCH₃)Me
Ph
2-F—Ph
2-Cl—Ph
3-F—Ph
4-F—Ph
4-Cl—Ph
4-OMe—Ph
4-CF₃—Ph
4-CN—Ph
5-OMe-3-pyridinyl
5-Cl-2-pyridinyl
6-OMe-3-pyridinyl
5-Cl-1,2,4-oxadiazol-3-yl
2-pyridinyl
CH(OH)CH₃
CH(OH)CH₂CH₃
CH(OH)CH(CH₃)₂
C(OH)(CH₃)₃
R² is 2-Cl, 3,5-di-OMe—Ph; (R⁵)ₘ is 2-F; W and
Y are both a direct bond.
CO₂Me
CO₂Et
CO₂-n-Pr
C(═O)Me
C(═O)Et
Me
Et
i-Pr
c-Pr
s-Bu
c-pentyl
c-hexyl
C(═CH₂)Me
C(═CH₂)Et
CH₂Cl
CH₂CN
CH═CH₂
C≡CH
Cl
C(═CHCH₃)Me
Ph
2-F—Ph
2-Cl—Ph
3-F—Ph
4-F—Ph
4-Cl—Ph
4-OMe—Ph
4-CF₃—Ph
4-CN—Ph
5-OMe-2-pyridinyl
5-Cl-2-pyridinyl
6-OMe-3-pyridinyl
5-Cl-1,2,4-oxadiazol-3-yl
2-pyridinyl
CH(OH)CH₃
CH(OH)CH₂CH₃
CH(OH)CH(CH₃)₂
C(OH)(CH₃)₃
R² is 2-Cl, 3,5-di-OMe—Ph; (R⁵)ₘ is 4-F; W and
Y are both a direct bond.
CO₂Me
CO₂Et
CO₂-n-Pr
C(═O)Me
C(═O)Et
Me
Et
i-Pr
c-Pr
s-Bu
c-pentyl
c-hexyl
C(═CH₂)Me
C(═CH₂)Et
CH₂Cl
CH₂CN
CH═CH₂
C≡CH
Cl
C(═CHCH₃)Me
Ph
2-F—Ph
2-Cl—Ph
3-F—Ph
4-F—Ph
4-Cl—Ph
4-OMe—Ph
4-CF₃—Ph
4-CN—Ph
5-OMe-2-pyridinyl
5-Cl-2-pyridinyl
6-OMe-3-pyridinyl
5-Cl-1,2,4-oxadiazol-3-yl
2-pyridinyl
CH(OH)CH₃
CH(OH)CH₂CH₃
CH(OH)CH(CH₃)₂
C(OH)(CH₃)₃
R² is 2-Cl, 3,5-di-OMe—Ph; (R⁵)ₘ is 2,4-di-F;
W and Y are both a direct bond.
CO₂Me
CO₂Et
CO₂-n-Pr
C(═O)Me
C(═O)Et
Me
Et
i-Pr
c-Pr
s-Bu
c-pentyl
c-hexyl
C(═CH₂)Me
C(═CH₂)Et
CH₂Cl
CH₂CN
CH═CH₂
C≡CH
Cl
C(═CHCH₃)Me
Ph
2-F—Ph
2-Cl—Ph
3-F—Ph
4-F—Ph
4-Cl—Ph
4-OMe—Ph
4-CF₃—Ph
4-CN—Ph
5-OMe-2-pyridinyl
5-Cl-2-pyridinyl
6-OMe-3-pyridinyl
5-Cl-1,2,4-oxadiazol-3-yl
2-pyridinyl
CH(OH)CH₃
CH(OH)CH₂CH₃
CH(OH)CH(CH₃)₂
C(OH)(CH₃)₃
R² is 2-Cl, 3,5-di-OMe—Ph; (R⁵)ₘ is 2,6-di-F;
W and Y are both a direct bond.
CO₂Me
CO₂Et
CO₂-n-Pr
C(═O)Me
C(═O)Et
Me
Et
i-Pr
c-Pr
s-Bu
c-pentyl
c-hexyl
C(═CH₂)Me
C(═CH₂)Et
CH₂Cl
CH₂CN
CH═CH₂
C≡CH
Cl
C(═CHCH₃)Me
Ph
2-F—Ph
2-Cl—Ph
3-F—Ph
4-F—Ph
4-Cl—Ph
4-OMe—Ph
4-CF₃—Ph
4-CN—Ph
5-OMe-2-pyridinyl
5-Cl-2-pyridinyl
6-OMe-3-pyridinyl
5-Cl-1,2,4-oxadiazol-3-yl
2-pyridinyl
CH(OH)CH₃
CH(OH)CH₂CH₃
CH(OH)CH(CH₃)₂
C(OH)(CH₃)₃
R² is 2-Cl, 3,5-di-OMe—Ph; (R⁵)ₘ is 2,4,6-tri-F;
W and Y are both a direct bond.
CO₂Me
CO₂Et
CO₂-n-Pr
C(═O)Me
C(═O)Et
Me
Et
i-Pr
c-Pr
s-Bu
c-pentyl
c-hexyl
C(═CH₂)Me
C(═CH₂)Et
CH₂Cl
CH₂CN
CH═CH₂
C≡CH
Cl
C(═CHCH₃)Me
Ph
2-F—Ph
2-Cl—Ph
3-F—Ph
4-F—Ph
4-Cl—Ph
4-OMe—Ph
4-CF₃—Ph
4-CN—Ph
5-OMe-2-pyridinyl
5-Cl-2-pyridinyl
6-OMe-3-pyridinyl
5-Cl-1,2,4-oxadiazol-3-yl
2-pyridinyl
CH(OH)CH₃
CH(OH)CH₂CH₃
CH(OH)CH(CH₃)₂
C(OH)(CH₃)₃
R² is 2-Cl, 3,5-di-OMe—Ph; (R⁵)ₘ is 4-Cl; W
and Y are both a direct bond.
CO₂Me
CO₂Et
CO₂-n-Pr
C(═O)Me
C(═O)Et
Me
Et
i-Pr
c-Pr
s-Bu
c-pentyl
c-hexyl
C(═CH₂)Me
C(═CH₂)Et
CH₂Cl
CH₂CN
CH═CH₂
C≡CH
Cl
C(═CHCH₃)Me
Ph
2-F—Ph
2-Cl—Ph
3-F—Ph
4-F—Ph
4-Cl—Ph
4-OMe—Ph
4-CF₃—Ph
4-CN—Ph
5-OMe-2-pyridinyl
5-Cl-2-pyridinyl
6-OMe-3-pyridinyl
5-Cl-1,2,4-oxadiazol-3-yl
2-pyridinyl
CH(OH)CH₃
CH(OH)CH₂CH₃
CH(OH)CH(CH₃)₂
C(OH)(CH₃)₃
R² is 2-Cl, 3,5-di-OMe—Ph; (R⁵)ₘ is 4-OMe; W
and Y are both a direct bond.
CO₂Me
CO₂Et
CO₂-n-Pr
C(═O)Me
C(═O)Et
Me
Et
i-Pr
c-Pr
s-Bu
c-pentyl
c-hexyl
C(═CH₂)Me
C(═CH₂)Et
CH₂Cl
CH₂CN
CH═CH₂
C≡CH
Cl
C(═CHCH₃)Me
Ph
2-F—Ph
2-Cl—Ph
3-F—Ph
4-F—Ph
4-Cl—Ph
4-OMe—Ph
4-CF₃—Ph
4-CN—Ph
5-OMe-2-pyridinyl
5-Cl-2-pyridinyl
6-OMe-3-pyridinyl
5-Cl-1,2,4-oxadiazol-3-yl
2-pyridinyl
CH(OH)CH₃
CH(OH)CH₂CH₃
CH(OH)CH(CH₃)₂
C(OH)(CH₃)₃
R² is 2-Cl, 3,5-di-OMe—Ph; (R⁵)ₘ is 2,3,6-tri-F;
W and Y are both a direct bond.
CO₂Me
CO₂Et
CO₂-n-Pr
C(═O)Me
C(═O)Et
Me
Et
i-Pr
c-Pr
s-Bu
c-pentyl
c-hexyl
C(═CH₂)Me
C(═CH₂)Et
CH₂Cl
CH₂CN
CH═CH₂
C≡CH
Cl
C(═CHCH₃)Me
Ph
2-F—Ph
2-Cl—Ph
3-F—Ph
4-F—Ph
4-Cl—Ph
4-OMe—Ph
4-CF₃—Ph
4-CN—Ph
5-OMe-2-pyridinyl
5-Cl-2-pyridinyl
6-OMe-3-pyridinyl
5-Cl-1,2,4-oxadiazol-3-yl
2-pyridinyl
CH(OH)CH₃
CH(OH)CH₂CH₃
CH(OH)CH(CH₃)₂
C(OH)(CH₃)₃
R² is 2-Cl, 3,5-di-OMe—Ph; (R⁵)ₘ is 2,6-di-F,
4-OMe; W and Y are both a direct bond.
CO₂Me
CO₂Et
CO₂-n-Pr
C(═O)Me
C(═O)Et
Me
Et
i-Pr
c-Pr
s-Bu
c-pentyl
c-hexyl
C(═(CH₂)Me
C(═CH₂)Et
CH₂Cl
CH₂CN
CH═CH₂
C≡CH
Cl
C(═CHCH₃)Me
Ph
2-F—Ph
2-Cl—Ph
3-F—Ph
4-F—Ph
4-Cl—Ph
4-OMe—Ph
4-CF₃—Ph
4-CN—Ph
5-OMe-2-pyridinyl
5-Cl-2-pyridinyl
6-OMe-3-pyridinyl
5-Cl-1,2,4-oxadiazol-3-yl
2-pyridinyl
CH(OH)CH₃
CH(OH)CH₂CH₃
CH(OH)CH(CH₃)₂
C(OH)(CH₃)₃
R² is 2-Cl, 3,5-di-OMe—Ph; (R⁵)ₘ is 2,6-di-F,
4-O(CH₂)₃NHCH₃; W and Y are both a direct
bond.
CO₂Me
CO₂Et
CO₂-n-Pr
C(═O)Me
C(═O)Et
Me
Et
i-Pr
c-Pr
s-Bu
c-pentyl
c-hexyl
C(═CH₂)Me
C(═CH₂)Et
CH₂Cl
CH₂CN
CH═CH₂
C≡CH
Cl
C(═CHCH₃)Me
Ph
2-F—Ph
2-Cl—Ph
3-F—Ph
4-F—Ph
4-Cl—Ph
4-OMe—Ph
4-CF₃—Ph
4-CN—Ph
5-OMe-2-pyridinyl
5-Cl-2-pyridinyl
6-OMe-3-pyridinyl
5-Cl-1,2,4-oxadiazol-3-yl
2-pyridinyl
CH(OH)CH₃
CH(OH)CH₂CH₃
CH(OH)CH(CH₃)₂
C(OH)(CH₃)₃

TABLE 2

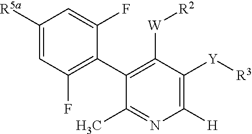

| | | |
|---|---|---|
| R² | R³ | R^{5a} |

W is O; and Y is a direct bond.

| | | |
|---|---|---|
| 3,5-di-OM—Ph | Ph | H |
| 2-Cl, 3,5-di-OMe—Ph | Ph | H |
| 3,5-di-OMe—Ph | 2-F—Ph | H |
| 2-Cl, 3,5-di-OMe—Ph | 2-F—Ph | H |
| 3,5-di-OMe—Ph | 4-F—Ph | H |
| 2-Cl, 3,5-di-OMe—Ph | 4-F—Ph | H |
| 3,5-di-OMe—Ph | 4-Cl—Ph | H |
| 2-Cl, 3,5-di-OMe—Ph | 4-Cl—Ph | H |
| 3,5-di-OMe—Ph | i-Pr | H |
| 2-Cl, 3,5-di-OMe—Ph | i-Pr | H |
| 3,5-di-OMe—Ph | C(═CH₂)Me | H |
| 2-Cl, 3,5-di-OMe—Ph | C(═CH₂)Me | H |
| 3,5-di-OMe—Ph | Ph | F |
| 2-Cl, 3,5-di-OMe—Ph | Ph | F |
| 3,5-di-OMe—Ph | 2-F—Ph | F |
| 2-Cl, 3,5-di-OMe—Ph | 2-F—Ph | F |
| 3,5-di-OMe—Ph | 4-F—Ph | F |
| 2-Cl, 3,5-di-OMe—Ph | 4-F—Ph | F |
| 3,5-di-OMe—Ph | 4-Cl—Ph | F |
| 2-Cl, 3,5-di-OMe—Ph | 4-Cl—Ph | F |
| 3,5-di-OMe—Ph | i-Pr | F |
| 2-Cl, 3,5-di-OMe—Ph | i-Pr | F |
| 3,5-di-OMe—Ph | C(═CH₂)Me | F |
| 2-Cl, 3,5-di-OMe—Ph | C(═CH₂)Me | F |

W is S; and Y is a direct bond.

| | | |
|---|---|---|
| 3,5-di-OMe—Ph | Ph | H |
| 2-Cl, 3,5-di-OMe—Ph | Ph | H |
| 3,5-di-OMe—Ph | 2-F—Ph | H |
| 2-Cl, 3,5-di-OMe—Ph | 2-F—Ph | H |
| 3,5-di-OMe—Ph | 4-F—Ph | H |
| 2-Cl, 3,5-di-OMe—Ph | 4-F—Ph | H |
| 3,5-di-OMe—Ph | 4-Cl—Ph | H |
| 2-Cl, 3,5-di-OMe—Ph | 4-Cl—Ph | H |
| 3,5-di-OMe—Ph | i-Pr | H |
| 2-Cl, 3,5-di-OMe—Ph | i-Pr | H |
| 3,5-di-OMe—Ph | C(═CH₂)Me | H |
| 2-Cl, 3,5-di-OMe—Ph | C(═CH₂)Me | H |
| 3,5-di-OMe—Ph | Ph | F |
| 2-Cl, 3,5-di-OMe—Ph | Ph | F |
| 3,5-di-OMe—Ph | 2-F—Ph | F |
| 2-Cl, 3,5-di-OMe—Ph | 2-F—Ph | F |
| 3,5-di-OMe—Ph | 4-F—Ph | F |
| 2-Cl, 3,5-di-OMe—Ph | 4-F—Ph | F |
| 3,5-di-OMe—Ph | 4-Cl—Ph | F |
| 2-Cl, 3,5-di-OMe—Ph | 4-Cl—Ph | F |
| 3,5-di-OMe—Ph | i-Pr | F |
| 2-Cl, 3,5-di-OMe—Ph | i-Pr | F |
| 3,5-di-OMe—Ph | C(═CH₂)Me | F |
| 2-Cl, 3,5-di-OMe—Ph | C(═CH₂)Me | F |

TABLE 3

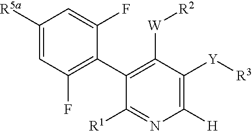

R³
R¹ is Cl; R² is 3,5-di-OMe—Ph; R⁵a is H; W
and Y are both a direct bond.
CO₂Me
CO₂Et
CO₂-n-Pr
C(═O)Me
C(═O)Et
Me
Et
i-Pr
c-Pr
s-Bu
c-pentyl
c-hexyl
C(═CH₂)Me
C(═CH₂)Et
CH₂Cl
CH₂CN
CH═CH₂
C≡CH
Cl
C(═CHCH₃)Me
Ph
2-F—Ph
2-Cl—Ph
3-F—Ph
4-F—Ph
4-Cl—Ph
4-OMe—Ph
4-CF₃—Ph
4-CN—Ph
5-OMe-2-pyridinyl
5-Cl-2-pyridinyl
6-OMe-3-pyridinyl
5-Cl-1,2,4-oxadiazol-3-yl
2-pyridinyl
CH(OH)CH₃
CH(OH)CH₂CH₃
CH(OH)CH(CH₃)₂
C(OH)CH₃)₃
R¹ is Cl; R² is 3,5-di-OMe—Ph; R⁵a is F; W
and Y are both a direct bond.
CO₂Me
CO₂Et
CO₂-n-Pr
C(═O)Me
C(═O)Et
Me
Et
i-Pr
c-Pr
s-Bu
c-pentyl
c-hexyl
C(═CH₂)Me
C(═CH₂)Et
CH₂Cl
CH₂CN
CH═CH₂
C≡CH
Cl
C(═CHCH₃)Me
Ph
2-F—Ph
2-Cl—Ph
3-F—Ph
4-F—Ph
4-Cl—Ph
4-OMe—Ph
4-CF₃—Ph
4-CN—Ph
5-OMe-2-pyridinyl
5-Cl-2-pyridinyl
6-OMe-3-pyridinyl
5-Cl-1,2,4-oxadiazol-3-yl
2-pyridinyl
CH(OH)CH₃
CH(OH)CH₂CH₃
CH(OH)CH(CH₃)₂
C(OH)(CH₃)₃
R¹ is CN; R² is 3,5-di-OMe—Ph; R⁵a is H; W
and Y are both a direct bond.
CO₂Me
CO₂Et
CO₂-n-Pr
C(═O)Me
C(═O)Et
Me
Et
i-Pr
c-Pr
s-Bu
c-pentyl
c-hexyl
C(═CH₂)Me
C(═CH₂)Et
CH₂Cl
CH₂CN
CH═CH₂
C≡CH
Cl
C(═CHCH₃)Me
Ph
2-F—Ph
2-Cl—Ph
3-F—Ph
4-F—Ph
4-Cl—Ph
4-OMe—Ph
4-CF₃—Ph
4-CN—Ph
4-OMe-2-pyridinyl
4-Cl-2-pyridinyl
6-OMe-3-pyridinyl
5-Cl-1,2,4-oxadiazol-3-yl
2-pyridinyl
CH(OH)CH₃
CH(OH)CH₂CH₃
CH(OH)CH(CH₃)₂
C(OH)(CH₃)₃
R¹ is CN; R² is 3,5-di-OMe—Ph; R⁵a is F; W
and Y are both a direct bond.
CO₂Me
CO₂Et
CO₂-n-Pr
C(═O)Me
C(═O)Et
Me
Et
i-Pr
c-Pr
s-Bu
c-pentyl
c-hexyl
C(═CH₂)Me
C(═CH₂)Et
CH₂Cl
CH₂CN
CH═CH₂
C≡CH
Cl
C(═CHCH₃)Me
Ph
2-F—Ph
2-Cl—Ph
3-F—Ph
4-F—Ph
4-Cl—Ph
4-OMe—Ph
4-CF₃—Ph
4-CN—Ph
5-OMe-2-pyridinyl
5-Cl-2-pyridinyl
6-OMe-3-pyridinyl
5-Cl-1,2,4-oxadiazol-3-yl
2-pyridinyl
CH(OH)CH₃
CH(OH)CH₂CH₃
CH(OH)CH(CH₃)₂
C(OH)(CH₃)₃
R¹ is Et; R² is 3,5-di—Ph; R⁵a is H; W
and Y are both a direct bond.
CO₂Me
CO₂Et
CO₂-n-Pr
C(═O)Me
C(═O)Et
Me
Et
i-Pr
c-Pr
s-Bu
c-pentyl
c-hexyl
C(═CH₂)Me
C(═CH₂)Et
CH₂Cl
CH₂CN
CH═CH₂
CH≡CH
Cl
C(═CHCH₃)Me
Ph
2-F—Ph
2-Cl—Ph
3-F—Ph
4-F—Ph
4-Cl—Ph
4-OMe—Ph
4-CF₃—Ph
4-CN—Ph
5-OMe-2-pyridinyl
5-Cl-2-pyridinyl
6-OMe-3-pyridinyl
5-Cl-1,2,4-oxadiazol-3-yl
2-pyridinyl
CH(OH)CH₃
CH(OH)CH₂CH₃
CH(OH)CH(CH₃)₂
C(OH)(CH₃)₃
R¹ is Et; R² is 3,5-di-OMe—Ph; R⁵a is F; W
and Y are both a direct bond.
CO₂Me
CO₂Et
CO₂-n-Pr
C(═O)Me
C(═O)Et
Me
Et
i-Pr
c-Pr
s-Bu
c-pentyl
c-hexyl
C(═CH₂)Me
C(═CH₂)Et
CH₂Cl
CH₂CN
CH═CH₂
C≡CH
Cl
C(═CHCH₃)Me
Ph
2-F—Ph
2-Cl—Ph
3-F—Ph
4-F—Ph
4-Cl—Ph
4-OMe—Ph
4-CF₃—Ph
4-CN—Ph
5-OMe-2-pyridinyl
5-Cl-2-pyridinyl
6-OMe-3-pyridinyl
5-Cl-1,2,4-oxadiazol-3-yl
2-pyridinyl
CH(OH)CH₃
CH(OH)CH₂CH₃
CH(OH)CH(CH₃)₂
C(OH)(CH₃)₃
R¹ is Cl; R² is 2-Cl, 3,5-di-OMe—Ph; R⁵a is H;
W and Y are both a direct bond.
CO₂Me
CO₂Et
CO₂-n-Pr
C(═O)Me
C(═O)Et
Me
Et
i-Pr
c-Pr
s-Bu
c-pentyl
c-hexyl
C(═CH₂)Me
C(═CH₂)Et
CH₂Cl
CH₂CN
CH═CH₂
C≡CH
Cl
C(═CHCH₃)Me
Ph
2-F—Ph
2-Cl—Ph
3-F—Ph
4-F—Ph
4-Cl—Ph
4-OMe—Ph
4-CF₃—Ph
4-CN—Ph
5-OMe-2-pyridinyl
5-Cl-2-pyridinyl
6-OMe-3-pyridinyl
5-Cl-1,2,4-oxadiazol-3-yl
2-pyridinyl
CH(OH)CH₃
CH(OH)CH₂CH₃
CH(OH)CH(CH₃)₂
C(OH)(CH₃)₃
R¹ is Cl; R² is 2-Cl, 3,5-di-OMe—Ph; R⁵a is F;
W and Y are both a direct bond.
CO₂Me
CO₂Et
CO₂-n-Pr
C(═O)Me
C(═O)Et
Me
Et
i-Pr
c-Pr
s-Bu
c-pentyl
c-hexyl
C(═CH₂)Me
C(═CH₂)Et
CH₂Cl
CH₂CN
CH═CH₂
C≡CH
Cl
C(═CHCH₃)Me
Ph
2-F—Ph
2-Cl—Ph
3-F—Ph
4-F—Ph
4-Cl—Ph
4-OMe—Ph
4-CF₃—Ph
4-CN—Ph
5-OMe-2-pyridinyl
5-Cl-2-pyridinyl
6-OMe-3-pyridinyl
5-Cl-1,2,4-oxadiazol-3-yl
2-pyridinyl
CH(OH)CH₃
CH(OH)CH₂CH₃
CH(OH)CH(CH₃)₂
C(OH)(CH₃)₃
R¹ is CN; R² is 2-Cl, 3,5-di-OMe—Ph; R⁵a is H;
W and Y are both a direct bond.
CO₂Me
CO₂Et
CO₂-n-Pr
C(═O)Me
C(═O)Et
Me
Et
i-Pr
c-Pr
s-Bu
c-pentyl
c-hexyl
C(═CH₂)Me
C(═CH₂)Et
CH₂Cl
CH₂CN
CH═CH₂
C≡CH
Cl
C(═CHCH₃)Me
Ph
2-F—Ph
2-Cl—Ph
3-F—Ph
4-F—Ph
4-Cl—Ph
4-OMe—Ph
4-CF₃—Ph
4-CN—Ph
5-OMe-2-pyridinyl
5-Cl-2-pyridinyl
6-OMe-3-pyridinyl
5-Cl-1,2,4-oxadiazol-3-yl
2-pyridinyl
CH(OH)CH₃
CH(OH)CH₂CH₃
CH(OH)CH(CH₃)₂
C(OH)CH₃)₃
R¹ is CN; R² is 2-Cl, 3,5-di-OMe—Ph; R⁵a is F;
W and Y are both a direct bond.
CO₂Me
CO₂Et
CO₂-n-Pr
C(═O)Me
C(═O)Et
Me
Et
i-Pr
c-Pr
s-Bu
c-pentyl
c-hexyl
C(═CH₂)Me
C(═CH₂)Et
CH₂Cl
CH₂CN
CH═CH₂
C≡CH
Cl
C(═CHCH₃)Me
Ph
2-F—Ph
2-Cl—Ph
3-F—Ph
4-F—Ph
4-Cl—Ph
4-OMe—Ph
4-CF₃—Ph
4-CN—Ph
5-OMe-2-pyridinyl
5-Cl-2-pyridinyl
6-OMe-3-pyridinyl
5-Cl-1,2,4-oxadiazol-3-yl
2-pyridinyl
CH(OH)CH₃
CH(OH)CH₂CH₃
CH(OH)CH(CH₃)₂
C(OH)(CH₃)₃
R¹ is Et; R² is 2-Cl, 3,5-di-OMe—Ph; R⁵a is H;
W and Y are both a direct bond.
CO₂Me
CO₂Et
CO₂-n-Pr
C(═O)Me
C(═O)Et
Me
Et
i-Pr
c-Pr
s-Bu
c-pentyl
c-hexyl
C(═CH₂)Me
C(═CH₂)Et
CH₂Cl
CH₂CN
CH═CH₂
C≡CH
Cl
C(═CHCH₃)Me
Ph
2-F—Ph
2-Cl—Ph
3-F—Ph
4-F—Ph
4-Cl—Ph
4-OMe—Ph
4-CF₃—Ph
4-CN—Ph
5-OMe-2-pyridinyl
5-Cl-2-pyridinyl
6-OMe-3-pyridinyl
5-Cl-1,2,4-oxadiazol-3-yl
2-pyridinyl
CH(OH)CH₃
CH(OH)CH₂CH₃
CH(OH)CH(CH₃)₂
C(OH)(CH₃)₃
R¹ is Et; R² is 2-Cl, 3,5-di-OMe—Ph; R⁵a is F;
W and Y are both a direct bond.
CO₂Me
CO₂Et
CO₂-n-Pr
C(═O)Me
C(═O)Et
Me
Et
i-Pr
c=Pr
s-Bu
c-pentyl
c-hexyl
C(═CH₂)Me
C(═CH₂)Et
CH₂Cl
CH₂CN
CH═CH₂
C≡CH
Cl
C(═CHCH₃)Me
Ph
2-F—Ph
2-Cl—Ph
3-F—Ph
4-F—Ph
4-Cl—Ph
4-OMe—Ph
4-CF₃—Ph
4-CN—Ph
5-OMe-2-pyridinyl
5-Cl-2-pyridinyl
6-OMe-3-pyridinyl
5-Cl-1,2,4-oxadiazol-3-yl
2-pyridinyl
CH(OH)CH₃
CH(OH)CH₂CH₃
CH(OH)CH(CH₃)₂
C(OH)(CH₃)₃

TABLE 4

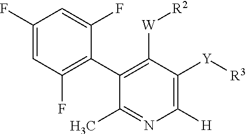

W and Y are both a direct bond.

| R² | R³ |
|---|---|
| 3-OMe—Ph | Ph |
| 2-F, 5-MeO—Ph | Ph |
| 2-Br, 5-OMe—Ph | Ph |
| 2-F, 3,5-di-OMe—Ph | Ph |
| 2-Cl, 5-OMe—Ph | Ph |
| 2-Br, 3,5-di-MeO—Ph | Ph |
| 3-OMe—Ph | 4-F—Ph |
| 2-F, 5-OMe—Ph | 4-F—Ph |
| 2-Br, 5-OMe—Ph | 4-F—Ph |
| 2-F, 3,5-di-OMe—Ph | 4-F—Ph |
| 2-Cl, 5-OMe—Ph | 4-F—Ph |
| 2-Br, 3,5-di-OMe—Ph | 4-F—Ph |
| 3-OMe—Ph | 2-F—Ph |
| 2-F, 5-OMe—Ph | 2-F—Ph |
| 2-Br, 5-OMe—Ph | 2-F—Ph |
| 2-F, 3,5-di-OMe—Ph | 2-F—Ph |
| 2-Cl, 5-OMe—Ph | 2-F—Ph |
| 2-Br, 3,5-di-OMe—Ph | 2-F—Ph |
| 3-OMe—Ph | i-Pr |
| 2-F, 5-MeO—Ph | i-Pr |
| 2-Br, 5-OMe—Ph | i-Pr |
| 2-F, 3,5-di-OMe—Ph | i-Pr |
| 2-Cl, 5-OMe—Ph | iPr |
| 2-Br, 3,5-di-MeO—Ph | i-Pr |
| 3-OMe—Ph | C(═CH₂)Me |
| 2-F, 5-OMe—Ph | C(═CH₂)Me |
| 2-Br, 5-OMe—Ph | C(═CH₂)Me |
| 2-F, 3,5-di-OMe—Ph | C(═CH₂)Me |
| 2-Cl, 5-OMe—Ph | C(═CH₂)Me |
| 2-Br, 3,5-di-OMe—Ph | C(═CH₂)Me |
| 3-OMe—Ph | CH₂CN |
| 2-F, 5-OMe—Ph | CH₂CN |
| 2-Br, 5-OMe—Ph | CH₂CN |
| 2-F, 3,5-di-OMe—Ph | CH₂CN |
| 2-Cl, 5-OMe—Ph | CH₂CN |
| 2-Br, 3,5-di-OMe—Ph | CH₂CN |

### Formulation/Utility

A compound of this invention will generally be used as a fungicidal active ingredient in a composition, i.e. formulation, with at least one additional component selected from the group consisting of surfactants, solid diluents and liquid diluents, which serves as a carrier. The formulation or composition ingredients are selected to be consistent with the physical properties of the active ingredient, mode of application and environmental factors such as soil type, moisture and temperature.

Useful formulations include both liquid and solid compositions. Liquid compositions include solutions (including emulsifiable concentrates), suspensions, emulsions (including microemulsions and/or suspoemulsions) and the like, which optionally can be thickened into gels. The general types of aqueous liquid compositions are soluble concentrate, suspension concentrate, capsule suspension, concentrated emulsion, microemulsion and suspo-emulsion. The general types of nonaqueous liquid compositions are emulsifiable concentrate, microemulsifiable concentrate, dispersible concentrate and oil dispersion.

The general types of solid compositions are dusts, powders, granules, pellets, prills, pastilles, tablets, filled films (including seed coatings) and the like, which can be water-dispersible (“wettable”) or water-soluble. Films and coatings formed from film-forming solutions or flowable suspensions are particularly useful for seed treatment. Active ingredient can be (micro)encapsulated and further formed into a suspension or solid formulation; alternatively the entire formulation of active ingredient can be encapsulated (or “overcoated”). Encapsulation can control or delay release of the active ingredient. An emulsifiable granule combines the advantages of both an emulsifiable concentrate formulation and a dry granular formulation. High-strength compositions are primarily used as intermediates for further formulation.

Sprayable formulations are typically extended in a suitable medium before spraying. Such liquid and solid formulations are formulated to be readily diluted in the spray medium, usually water. Spray volumes can range from about from about one to several thousand liters per hectare, but more typically are in the range from about ten to several hundred liters per hectare. Sprayable formulations can be tank mixed with water or another suitable medium for foliar treatment by aerial or ground application, or for application to the growing medium of the plant. Liquid and dry formulations can be metered directly into drip irrigation systems or metered into the furrow during planting. Liquid and solid formulations can be applied onto seeds of crops and other desirable vegetation as seed treatments before planting to protect developing roots and other subterranean plant parts and/or foliage through systemic uptake.

The formulations will typically contain effective amounts of active ingredient, diluent and surfactant within the following approximate ranges which add up to 100 percent by weight.

| | Weight Percent | | |
|---|---|---|---|
| | Active | | |
| | Ingredient | Diluent | Surfactant |
| Water-Dispersible and Water- | 0.001-90 | 0-99.999 | 0-15 |
| soluble Granules, Tablets and | | | |
| Powders | | | |
| Oil Dispersions, Suspensions, | 1-50 | 40-99 | 0-50 |
| Emulsions, Solutions | | | |
| (including Emulsifiable | | | |
| Concentrates) | | | |
| Dusts | 1-25 | 70-99 | 0-5 |
| Granules and Pellets | 0.001-95 | 5-99.999 | 0-15 |
| High Strength Compositions | 90-99 | 0-10 | 0-2 |

Solid diluents include, for example, clays such as bentonite, montmorillonite, attapulgite and kaolin, gypsum, cellulose, titanium dioxide, zinc oxide, starch, dextrin, sugars (e.g., lactose, sucrose), silica, talc, mica, diatomaceous earth, urea, calcium carbonate, sodium carbonate and bicarbonate, and sodium sulfate. Typical solid diluents are described in Watkins et al., Handbook of Insecticide Dust Diluents and Carriers, 2nd Ed., Dorland Books, Caldwell, N.J.

Liquid diluents include, for example, water, N,N-dimethylalkanamides (e.g., N,N-dimethylformamide), limonene, dimethyl sulfoxide, N-alkylpyrrolidones (e.g., N-methylpyrrolidinone), ethylene glycol, triethylene glycol, propylene glycol, dipropylene glycol, polypropylene glycol, propylene carbonate, butylene carbonate, paraffins (e.g., white mineral oils, normal paraffins, isoparaffins), alkylbenzenes, alkylnaphthalenes, glycerine, glycerol triacetate, sorbitol, triacetin, aromatic hydrocarbons, dearomatized aliphatics, alkylbenzenes, alkylnaphthalenes, ketones such as cyclohexanone, 2-heptanone, isophorone and 4-hydroxy-4-methyl-2-pentanone, acetates such as isoamyl acetate, hexyl acetate, heptyl acetate, octyl acetate, nonyl acetate, tridecyl acetate and isobornyl acetate, other esters such as alkylated lactate esters, dibasic esters and γ-butyrolactone, and alcohols, which can be linear, branched, saturated or unsaturated, such as methanol, ethanol, n-propanol, isopropyl alcohol, n-butanol, isobutyl alcohol, n-hexanol, 2-ethylhexanol, n-octanol, decanol, isodecyl alcohol, isooctadecanol, cetyl alcohol, lauryl alcohol, tridecyl alcohol, oleyl alcohol, cyclohexanol, tetrahydrofurfuryl alcohol, diacetone alcohol and benzyl alcohol. Liquid diluents also include glycerol esters of saturated and unsaturated fatty acids (typically C₆-C₂₂), such as plant seed and fruit oils (e.g., oils of olive, castor, linseed, sesame, corn (maize), peanut, sunflower, grapeseed, safflower, cottonseed, soybean, rapeseed, coconut and palm kernel), animal-sourced fats (e.g., beef tallow, pork tallow, lard, cod liver oil, fish oil), and mixtures thereof. Liquid diluents also include alkylated fatty acids (e.g., methylated, ethylated, butylated) wherein the fatty acids may be obtained by hydrolysis of glycerol esters from plant and animal sources, and can be purified by distillation. Typical liquid diluents are described in Marsden, Solvents Guide, 2nd Ed., Interscience, New York, 1950.

The solid and liquid compositions of the present invention often include one or more surfactants. When added to a liquid, surfactants (also known as “surface-active agents”) generally modify, most often reduce, the surface tension of the liquid. Depending on the nature of the hydrophilic and lipophilic groups in a surfactant molecule, surfactants can be useful as wetting agents, dispersants, emulsifiers or defoaming agents.

Surfactants can be classified as nonionic, anionic or cationic. Nonionic surfactants useful for the present compositions include, but are not limited to: alcohol alkoxylates such as alcohol alkoxylates based on natural and synthetic alcohols (which may be branched or linear) and prepared from the alcohols and ethylene oxide, propylene oxide, butylene oxide or mixtures thereof; amine ethoxylates, alkanolamides and ethoxylated alkanolamides; alkoxylated triglycerides such as ethoxylated soybean, castor and rapeseed oils; alkylphenol alkoxylates such as octylphenol ethoxylates, nonylphenol ethoxylates, dinonyl phenol ethoxylates and dodecyl phenol ethoxylates (prepared from the phenols and ethylene oxide, propylene oxide, butylene oxide or mixtures thereof); block polymers prepared from ethylene oxide or propylene oxide and reverse block polymers where the terminal blocks are prepared from propylene oxide; ethoxylated fatty acids; ethoxylated fatty esters and oils; ethoxylated methyl esters; ethoxylated tristyrylphenol (including those prepared from ethylene oxide, propylene oxide, butylene oxide or mixtures thereof); fatty acid esters, glycerol esters, lanolin-based derivatives, polyethoxylate esters such as polyethoxylated sorbitan fatty acid esters, polyethoxylated sorbitol fatty acid esters and polyethoxylated glycerol fatty acid esters; other sorbitan derivatives such as sorbitan esters; polymeric surfactants such as random copolymers, block copolymers, alkyd peg (polyethylene glycol) resins, graft or comb polymers and star polymers; polyethylene glycols (pegs); polyethylene glycol fatty acid esters; silicone-based surfactants; and sugar-derivatives such as sucrose esters, alkyl polyglycosides and alkyl polysaccharides.

Useful anionic surfactants include, but are not limited to: alkylaryl sulfonic acids and their salts; carboxylated alcohol or alkylphenol ethoxylates; diphenyl sulfonate derivatives; lignin and lignin derivatives such as lignosulfonates; maleic or succinic acids or their anhydrides; olefin sulfonates; phosphate esters such as phosphate esters of alcohol alkoxylates, phosphate esters of alkylphenol alkoxylates and phosphate esters of styryl phenol ethoxylates; protein-based surfactants; sarcosine derivatives; styryl phenol ether sulfate; sulfates and sulfonates of oils and fatty acids; sulfates and sulfonates of ethoxylated alkylphenols; sulfates of alcohols; sulfates of ethoxylated alcohols; sulfonates of amines and amides such as N,N-alkyltaurates; sulfonates of benzene, cumene, toluene, xylenes, and dodecyl and tridecylbenzenes; sulfonates of condensed naphthalenes; sulfonates of naphthalene and alkyl naphthalene; sulfonates of fractionated petroleum; sulfosuccinamates; and sulfosuccinates and their derivatives such as dialkyl sulfosuccinate salts.

Useful cationic surfactants include, but are not limited to: amides and ethoxylated amides; amines such as N-alkyl propanediamines, tripropylenetriamines and dipropylenetetramines, and ethoxylated amines, ethoxylated diamines and propoxylated amines (prepared from the amines and ethylene oxide, propylene oxide, butylene oxide or mixtures thereof); amine salts such as amine acetates and diamine salts; quaternary ammonium salts such as quaternary salts, ethoxylated quaternary salts and diquaternary salts; and amine oxides such as alkyldimethylamine oxides and bis-(2-hydroxyethyl)-alkylamine oxides.

Also useful for the present compositions are mixtures of nonionic and anionic surfactants or mixtures of nonionic and cationic surfactants. Nonionic, anionic and cationic surfactants and their recommended uses are disclosed in a variety of published references including McCutcheon's Emulsifiers and Detergents, annual American and International Editions published by McCutcheon's Division, The Manufacturing Confectioner Publishing Co.; Sisely and Wood, Encyclopedia of Surface Active Agents, Chemical Publ. Co., Inc., New York, 1964; and A. S. Davidson and B. Milwidsky, Synthetic Detergents, Seventh Edition, John Wiley and Sons, New York, 1987.

Compositions of this invention may also contain formulation auxiliaries and additives, known to those skilled in the art as formulation aids (some of which may be considered to also function as solid diluents, liquid diluents or surfactants). Such formulation auxiliaries and additives may control: pH (buffers), foaming during processing (antifoams such polyorganosiloxanes), sedimentation of active ingredients (suspending agents), viscosity (thixotropic thickeners), in-container microbial growth (antimicrobials), product freezing (antifreezes), color (dyes/pigment dispersions), wash-off (film formers or stickers), evaporation (evaporation retardants), and other formulation attributes. Film formers include, for example, polyvinyl acetates, polyvinyl acetate copolymers, polyvinylpyrrolidone-vinyl acetate copolymer, polyvinyl alcohols, polyvinyl alcohol copolymers and waxes. Examples of formulation auxiliaries and additives include those listed in McCutcheon's Volume 2: Functional Materials, annual International and North American editions published by McCutcheon's Division, The Manufacturing Confectioner Publishing Co.; and PCT Publication WO 03/024222.

The compound of Formula 1 and any other active ingredients are typically incorporated into the present compositions by dissolving the active ingredient in a solvent or by grinding in a liquid or dry diluent. Solutions, including emulsifiable concentrates, can be prepared by simply mixing the ingredients. If the solvent of a liquid composition intended for use as an emulsifiable concentrate is water-immiscible, an emulsifier is typically added to emulsify the active-containing solvent upon dilution with water. Active ingredient slurries, with particle diameters of up to 2,000 μm can be wet milled using media mills to obtain particles with average diameters below 3 μm. Aqueous slurries can be made into finished suspension concentrates (see, for example, U.S. Pat. No. 3,060,084) or further processed by spray drying to form water-dispersible granules. Dry formulations usually require dry milling processes, which produce average particle diameters in the 2 to 10 μm range. Dusts and powders can be prepared by blending and usually grinding (such as with a hammer mill or fluid-energy mill). Granules and pellets can be prepared by spraying the active material upon preformed granular carriers or by agglomeration techniques. See Browning, “Agglomeration”, Chemical Engineering, Dec. 4, 1967, pp 147-48, Perry's Chemical Engineer's Handbook, 4th Ed., McGraw-Hill, New York, 1963, pages 8-57 and following, and WO 91/13546. Pellets can be prepared as described in U.S. Pat. No. 4,172,714. Water-dispersible and water-soluble granules can be prepared as taught in U.S. Pat. No. 4,144,050, U.S. Pat. No. 3,920,442 and DE 3,246,493. Tablets can be prepared as taught in U.S. Pat. No. 5,180,587, U.S. Pat. No. 5,232,701 and U.S. Pat. No. 5,208,030. Films can be prepared as taught in GB 2,095,558 and U.S. Pat. No. 3,299,566.

For further information regarding the art of formulation, see T. S. Woods, “The Formulator's Toolbox—Product Forms for Modern Agriculture” in Pesticide Chemistry and Bioscience, The Food-Environment Challenge, T. Brooks and T. R. Roberts, Eds., Proceedings of the 9th International Congress on Pesticide Chemistry, The Royal Society of Chemistry, Cambridge, 1999, pp. 120-133. See also U.S. Pat. No. 3,235,361, Col. 6, line 16 through Col. 7, line 19 and Examples 10-41; U.S. Pat. No. 3,309,192, Col. 5, line 43 through Col. 7, line 62 and Examples 8, 12, 15, 39, 41, 52, 53, 58, 132, 138-140, 162-164, 166, 167 and 169-182; U.S. Pat. No. 2,891,855, Col. 3, line 66 through Col. 5, line 17 and Examples 1-4; Klingman, Weed Control as a Science, John Wiley and Sons, Inc., New York, 1961, pp 81-96; Hance et al., Weed Control Handbook, 8th Ed., Blackwell Scientific Publications, Oxford, 1989; and Developments in formulation technology, PJB Publications, Richmond, UK, 2000.

In the following Examples, all percentages are by weight and all formulations are prepared in conventional ways. Compound numbers refer to compounds in Index Table A. Without further elaboration, it is believed that one skilled in the art using the preceding description can utilize the present invention to its fullest extent. The following Examples are, therefore, to be constructed as merely illustrative, and not limiting of the disclosure in any way whatsoever. Percentages are by weight except where otherwise indicated.

### Example A

### High Strength Concentrate

| | |
|---|---|
| Compound 72 | 98.5% |
| silica aerogel | 0.5% |
| synthetic amorphous fine silica | 1.0% |

### Example B

| | |
|---|---|
| Compound 89 | 65.0% |
| dodecylphenol polyethylene glycol ether | 2.0% |
| sodium ligninsulfonate | 4.0% |
| sodium silicoaluminate | 6.0% |
| montmorillonite (calcined) | 23.0% |

### Example C

| | |
|---|---|
| Compound 121 | 10.0% |
| attapulgite granules (low volatile matter, 0.71/0.30 mm; | 90.0% |
| U.S.S. No. 25-50 sieves) | |

### Example D

### Extruded Pellet

| | |
|---|---|
| Compound 124 | 25.0% |
| anhydrous sodium sulfate | 10.0% |
| crude calcium ligninsulfonate | 5.0% |
| sodium alkylnaphthalenesulfonate | 1.0% |
| calcium/magnesium bentonite | 59.0% |

### Example E

### Emulsifiable Concentrate

| | |
|---|---|
| Compound 133 | 10.0% |
| polyoxyethylene sorbitol hexoleate | 20.0% |
| C₆-C₁₀ fatty acid methyl ester | 70.0% |

### Example F

### Microemulsion

| | |
|---|---|
| Compound 135 | 5.0% |
| polyvinylpyrrolidone-vinyl acetate copolymer | 30.0% |
| alkylpolyglycoside | 30.0% |
| glyceryl monooleate | 15.0% |
| water | 20.0% |

### Example G

### Seed Treatment

| | |
|---|---|
| Compound 137 | 20.00% |
| polyvinylpyrrolidone-vinyl acetate copolymer | 5.00% |
| montan acid wax | 5.00% |
| calcium ligninsulfonate | 1.00% |
| polyoxyethylene/polyoxypropylene block copolymers | 1.00% |
| stearyl alcohol (POE 20) | 2.00% |
| polyorganosilane | 0.20% |
| colorant red dye | 0.05% |
| water | 65.75% |

Water-soluble and water-dispersible formulations are typically diluted with water to form aqueous compositions before application. Aqueous compositions for direct applications to the plant or portion thereof (e.g., spray tank compositions) typically at least about 1 ppm or more (e.g., from 1 ppm to 100 ppm) of the compound(s) of this invention.

The compounds of this invention are useful as plant disease control agents. The present invention therefore further comprises a method for controlling plant diseases caused by fungal plant pathogens comprising applying to the plant or portion thereof to be protected, or to the plant seed to be protected, an effective amount of a compound of the invention or a fungicidal composition containing said compound. The compounds and/or compositions of this invention provide control of diseases caused by a broad spectrum of fungal plant pathogens in the Basidiomycete, Ascomycete, Oomycete and Deuteromycete classes. They are effective in controlling a broad spectrum of plant diseases, particularly foliar pathogens of ornamental, turf, vegetable, field, cereal, and fruit crops. These pathogens include: Oomycetes, including Phytophthora diseases such as Phytophthora infestans, Phytophthora megasperma, Phytophthora parasitica, Phytophthora cinnamomi and Phytophthora capsici, Pythium diseases such as Pythium aphanidermatum, and diseases in the Peronosporaceae family such as Plasmopara viticola, Peronospora spp. (including Peronospora tabacina and Peronospora parasitica), Pseudoperonospora spp. (including Pseudoperonospora cubensis) and Bremia lactucae; Ascomycetes, including Alternaria diseases such as Alternaria solani and Alternaria brassicae, Guignardia diseases such as Guignardia bidwell, Venturia diseases such as Venturia inaequalis, Septoria diseases such as Septoria nodorum and Septoria tritici, powdery mildew diseases such as Erysiphe spp. (including Erysiphe graminis and Erysiphe polygoni), Uncinula necatur, Sphaerotheca fuligena and Podosphaera leucotricha, Pseudocercosporella herpotrichoides, Botrytis diseases such as Botrytis cinerea, Monilinia fructicola, Sclerotinia diseases such as Sclerotinia sclerotiorum, Magnaporthe grisea, Phomopsis viticola, Helminthosporium diseases such as Helminthosporium tritici repentis, Pyrenophora teres, anthracnose diseases such as Glomerella or Colletotrichum spp. (such as Colletotrichum graminicola and Colletotrichum orbiculare), and Gaeumannomyces graminis; Basidiomycetes, including rust diseases caused by Puccinia spp. (such as Puccinia recondite, Puccinia striiformis, Puccinia hordei, Puccinia graminis and Puccinia arachidis), Hemileia vastatrix and Phakopsora pachyrhizi; other pathogens including Rutstroemia floccosum (also known as Sclerontina homoeocarpa); Rhizoctonia spp. (such as Rhizoctonia solani); Fusarium diseases such as Fusarium roseum, Fusarium graminearum and Fusarium oxysporum; Verticillium dahliae; Sclerotium rolfsii; Rynchosporium secalis; Cercosporidium personatum, Cercospora arachidicola and Cercospora beticola; and other genera and species closely related to these pathogens. In addition to their fungicidal activity, the compositions or combinations also have activity against bacteria such as Erwinia amylovora, Xanthomonas campestris, Pseudomonas syringae, and other related species.

Plant disease control is ordinarily accomplished by applying an effective amount of a compound of this invention either pre- or post-infection, to the portion of the plant to be protected such as the roots, stems, foliage, fruit, seeds, tubers or bulbs, or to the media (soil or sand) in which the plants to be protected are growing. The compounds can also be applied to seeds to protect the seeds and seedlings developing from the seeds. The compounds can also be applied through irrigation water to treat plants.

Rates of application for these compounds (i.e. a fungicidally effective amount) can be influenced by many factors of the environment and should be determined under actual use conditions. One skilled in the art can easily determine through simple experimentation the fungicidally effective amount necessary for the desired level of plant disease control. Foliage can normally be protected when treated at a rate of from less than about 1 g/ha to about 5,000 g/ha of active ingredient. Seed and seedlings can normally be protected when seed is treated at a rate of from about 0.1 to about 10 g per kilogram of seed.

Compounds of this invention can also be mixed with one or more other biologically active compounds or agents including fungicides, insecticides, nematocides, bactericides, acaricides, herbicides, herbicide safeners, growth regulators such as insect molting inhibitors and rooting stimulants, chemosterilants, semiochemicals, repellents, attractants, pheromones, feeding stimulants, plant nutrients, other biologically active compounds or entomopathogenic bacteria, virus or fungi to form a multi-component pesticide giving an even broader spectrum of agricultural protection. Thus the present invention also pertains to a composition comprising a fungicidally effective amount of a compound of Formula 1 and a biologically effective amount of at least one additional biologically active compound or agent and can further comprise at least one of a surfactant, a solid diluent or a liquid diluent. The other biologically active compounds or agents can be formulated in compositions comprising at least one of a surfactant, solid or liquid diluent. For mixtures of the present invention, one or more other biologically active compounds or agents can be formulated together with a compound of Formula 1, to form a premix, or one or more other biologically active compounds or agents can be formulated separately from the compound of Formula 1, and the formulations combined together before application (e.g., in a spray tank) or, alternatively, applied in succession.

Of note is a composition which in addition to the compound of Formula 1 include at least one fungicidal compound selected from the group consisting of the classes (1) methyl benzimidazole carbamate (MBC) fungicides; (2) dicarboximide fungicides; (3) demethylation inhibitor (DMI) fungicides; (4) phenylamide fungicides; (5) amine/morpholine fungicides; (6) phospholipid biosynthesis inhibitor fungicides; (7) carboxamide fungicides; (8) hydroxy(2-amino-)pyrimidine fungicides; (9) anilinopyrimidine fungicides; (10) N-phenyl carbamate fungicides; (11) quinone outside inhibitor (QoI) fungicides; (12) phenylpyrrole fungicides; (13) quinoline fungicides; (14) lipid peroxidation inhibitor fungicides; (15) melanin biosynthesis inhibitors-reductase (MBI-R) fungicides; (16) melanin biosynthesis inhibitors-dehydratase (MBI-D) fungicides; (17) hydroxyanilide fungicides; (18) squalene-epoxidase inhibitor fungicides; (19) polyoxin fungicides; (20) phenylurea fungicides; (21) quinone inside inhibitor (QiI) fungicides; (22) benzamide fungicides; (23) enopyranuronic acid antibiotic fungicides; (24) hexopyranosyl antibiotic fungicides; (25) glucopyranosyl antibiotic: protein synthesis fungicides; (26) glucopyranosyl antibiotic: trehalase and inositol biosynthesis fungicides; (27) cyanoacetamideoxime fungicides; (28) carbamate fungicides; (29) oxidative phosphorylation uncoupling fungicides; (30) organo tin fungicides; (31) carboxylic acid fungicides; (32) heteroaromatic fungicides; (33) phosphonate fungicides; (34) phthalamic acid fungicides; (35) benzotriazine fungicides; (36) benzene-sulfonamide fungicides; (37) pyridazinone fungicides; (38) thiophene-carboxamide fungicides; (39) pyrimidinamide fungicides; (40) carboxylic acid amide (CAA) fungicides; (41) tetracycline antibiotic fungicides; (42) thiocarbamate fungicides; (43) benzamide fungicides; (44) host plant defense induction fungicides; (45) multi-site contact activity fungicides; (46) fungicides other than classes (1) through (45); and salts of compounds of classes (1) through (46).

Further descriptions of these classes of fungicidal compounds are provided below.

(1) “Methyl benzimidazole carbamate (MBC) fungicides” (Fungicide Resistance Action Committee (FRAC) code 1) inhibit mitosis by binding to β-tubulin during microtubule assembly. Inhibition of microtubule assembly can disrupt cell division, transport within the cell and cell structure. Methyl benzimidazole carbamate fungicides include benzimidazole and thiophanate fungicides. The benzimidazoles include benomyl, carbendazim, fuberidazole and thiabendazole. The thiophanates include thiophanate and thiophanate-methyl.

(2) “Dicarboximide fungicides” (Fungicide Resistance Action Committee (FRAC) code 2) are proposed to inhibit a lipid peroxidation in fungi through interference with NADH cytochrome c reductase. Examples include chlozolinate, iprodione, procymidone and vinclozolin.

(3) “Demethylation inhibitor (DMI) fungicides” (Fungicide Resistance Action Committee (FRAC) code 3) inhibit C14-demethylase, which plays a role in sterol production. Sterols, such as ergosterol, are needed for membrane structure and function, making them essential for the development of functional cell walls. Therefore, exposure to these fungicides results in abnormal growth and eventually death of sensitive fungi. DMI fungicides are divided between several chemical classes: azoles (including triazoles and imidazoles), pyrimidines, piperazines and pyridines. The triazoles include azaconazole, bitertanol, bromuconazole, cyproconazole, difenoconazole, diniconazole (including diniconazole-M), epoxiconazole, fenbuconazole, fluquinconazole, flusilazole, flutriafol, hexaconazole, imibenconazole, ipconazole, metconazole, myclobutanil, penconazole, propiconazole, prothioconazole, simeconazole, tebuconazole, tetraconazole, triadimefon, triadimenol, triticonazole and uniconazole. The imidazoles include clotrimazole, imazalil, oxpoconazole, prochloraz, pefurazoate and triflumizole. The pyrimidines include fenarimol and nuarimol. The piperazines include triforine. The pyridines include pyrifenox. Biochemical investigations have shown that all of the above mentioned fungicides are DMI fungicides as described by K. H. Kuck et al. in Modern Selective Fungicides—Properties, Applications and Mechanisms of Action, H. Lyr (Ed.), Gustav Fischer Verlag: New York, 1995, 205-258.

(4) “Phenylamide fungicides” (Fungicide Resistance Action Committee (FRAC) code

4) are specific inhibitors of RNA polymerase in Oomycete fungi. Sensitive fungi exposed to these fungicides show a reduced capacity to incorporate uridine into rRNA. Growth and development in sensitive fungi is prevented by exposure to this class of fungicide. Phenylamide fungicides include acylalanine, oxazolidinone and butyrolactone fungicides. The acylalanines include benalaxyl, benalaxyl-M, furalaxyl, metalaxyl and metalaxyl-M/mefenoxam. The oxazolidinones include oxadixyl. The butyrolactones include ofurace.

(5) “Amine/morpholine fungicides” (Fungicide Resistance Action Committee (FRAC) code 5) inhibit two target sites within the sterol biosynthetic pathway, Δ⁸→Δ⁷ isomerase and Δ¹⁴ reductase. Sterols, such as ergosterol, are needed for membrane structure and function, making them essential for the development of functional cell walls. Therefore, exposure to these fungicides results in abnormal growth and eventually death of sensitive fungi. Amine/morpholine fungicides (also known as non-DMI sterol biosynthesis inhibitors) include morpholine, piperidine and spiroketal-amine fungicides. The morpholines include aldimorph, dodemorph, fenpropimorph, tridemorph and trimorphamide. The piperidines include fenpropidin and piperalin. The spiroketal-amines include spiroxamine.

(6) “Phospholipid biosynthesis inhibitor fungicides” (Fungicide Resistance Action Committee (FRAC) code 6) inhibit growth of fungi by affecting phospholipid biosynthesis. Phospholipid biosynthesis fungicides include phosphorothiolate and dithiolane fungicides. The phosphorothiolates include edifenphos, iprobenfos and pyrazophos. The dithiolanes include isoprothiolane.

(7) “Carboxamide fungicides” (Fungicide Resistance Action Committee (FRAC) code 7) inhibit Complex II (succinate dehydrogenase) fungal respiration by disrupting a key enzyme in the Krebs Cycle (TCA cycle) named succinate dehydrogenase. Inhibiting respiration prevents the fungus from making ATP, and thus inhibits growth and reproduction. Carboxamide fungicides include benzamides, furan carboxamides, oxathiin carboxamides, thiazole carboxamides, pyrazole carboxamides and pyridine carboxamides. The benzamides include benodanil, flutolanil and mepronil. The furan carboxamides include fenfuram. The oxathiin carboxamides include carboxin and oxycarboxin. The thiazole carboxamides include thifluzamide. The pyrazole carboxamides include furametpyr, penthiopyrad, bixafen, isopyrazam, N-[2-(1S,2R)-[1,1′-bicyclopropyl]-2-ylphenyl]-3-(difluoromethyl)-1-methyl-1H-pyrazole-4-carboxamide and N-[2-(1,3-dimethyl-butyl)phenyl]-5-fluoro-1,3-dimethyl-1H-pyrazole-4-carboxamide. The pyridine carboxamides include boscalid.

(8) “Hydroxy(2-amino-)pyrimidine fungicides” (Fungicide Resistance Action Committee (FRAC) code 8) inhibit nucleic acid synthesis by interfering with adenosine deaminase. Examples include bupirimate, dimethirimol and ethirimol.

(9) “Anilinopyrimidine fungicides” (Fungicide Resistance Action Committee (FRAC) code 9) are proposed to inhibit biosynthesis of the amino acid methionine and to disrupt the secretion of hydrolytic enzymes that lyse plant cells during infection. Examples include cyprodinil, mepanipyrim and pyrimethanil.

(10) “N-Phenyl carbamate fungicides” (Fungicide Resistance Action Committee (FRAC) code 10) inhibit mitosis by binding to β-tubulin and disrupting microtubule assembly. Inhibition of microtubule assembly can disrupt cell division, transport within the cell and cell structure. Examples include diethofencarb.

(11) “Quinone outside inhibitor (QoI) fungicides” (Fungicide Resistance Action Committee (FRAC) code 11) inhibit Complex III mitochondrial respiration in fungi by affecting ubiquinol oxidase. Oxidation of ubiquinol is blocked at the “quinone outside” (Q_{O}) site of the cytochrome bc₁ complex, which is located in the inner mitochondrial membrane of fungi. Inhibiting mitochondrial respiration prevents normal fungal growth and development. Quinone outside inhibitor fungicides (also known as strobilurin fungicides) include methoxyacrylate, methoxycarbamate, oximinoacetate, oximinoacetamide, oxazolidinedione, dihydrodioxazine, imidazolinone and benzylcarbamate fungicides. The methoxyacrylates include azoxystrobin, enestroburin (SYP-Z071) and picoxystrobin. The methoxycarbamates include pyraclostrobin. The oximinoacetates include kresoxim-methyl and trifloxystrobin. The oximinoacetamides include dimoxystrobin, metominostrobin, orysastrobin, α-[methoxyimino]-N-methyl-2-[[[1-[3-(trifluoromethyl)phenyl]ethoxy]imino]-methyl]benzeneacetamide and 2-[[[3-(2,6-dichlorophenyl)-1-methyl-2-propen-1-ylidene]-amino]oxy]methyl]-α-(methoxyimino)-N-methylbenzeneacetamide. The oxazolidinediones include famoxadone. The dihydrodioxazines include fluoxastrobin. The imidazolinones include fenamidone. The benzylcarbamates include pyribencarb.

(12) “Phenylpyrrole fungicides” (Fungicide Resistance Action Committee (FRAC) code 12) inhibit a MAP protein kinase associated with osmotic signal transduction in fungi. Fenpiclonil and fludioxonil are examples of this fungicide class.

(13) “Quinoline fungicides” (Fungicide Resistance Action Committee (FRAC) code 13) are proposed to inhibit signal transduction by affecting G-proteins in early cell signaling. They have been shown to interfere with germination and/or appressorium formation in fungi that cause powder mildew diseases. Quinoxyfen is an example of this class of fungicide.

(14) “Lipid peroxidation inhibitor fungicides” (Fungicide Resistance Action Committee (FRAC) code 14) are proposed to inhibit lipid peroxidation which affects membrane synthesis in fungi. Members of this class, such as etridiazole, may also affect other biological processes such as respiration and melanin biosynthesis. Lipid peroxidation fungicides include aromatic carbon and 1,2,4-thiadiazole fungicides. The aromatic carbon fungicides include biphenyl, chloroneb, dicloran, quintozene, tecnazene and tolclofos-methyl. The 1,2,4-thiadiazole fungicides include etridiazole.

(15) “Melanin biosynthesis inhibitors-reductase (MBI-R) fungicides” (Fungicide Resistance Action Committee (FRAC) code 16.1) inhibit the naphthal reduction step in melanin biosynthesis. Melanin is required for host plant infection by some fungi. Melanin biosynthesis inhibitors-reductase fungicides include isobenzofuranone, pyrroloquinolinone and triazolobenzothiazole fungicides. The isobenzofuranones include fthalide. The pyrroloquinolinones include pyroquilon. The triazolobenzothiazoles include tricyclazole.

(16) “Melanin biosynthesis inhibitors-dehydratase (MBI-D) fungicides” (Fungicide Resistance Action Committee (FRAC) code 16.2) inhibit scytalone dehydratase in melanin biosynthesis. Melanin in required for host plant infection by some fungi. Melanin biosynthesis inhibitors-dehydratase fungicides include cyclopropanecarboxamide, carboxamide and propionamide fungicides. The cyclopropanecarboxamides include carpropamid. The carboxamides include diclocymet. The propionamides include fenoxanil.

(17) “Hydroxyanilide fungicides (Fungicide Resistance Action Committee (FRAC) code 17) inhibit C4-demethylase which plays a role in sterol production. Examples include fenhexamid.

(18) “Squalene-epoxidase inhibitor fungicides” (Fungicide Resistance Action Committee (FRAC) code 18) inhibit squalene-epoxidase in ergosterol biosynthesis pathway. Sterols such as ergosterol are needed for membrane structure and function, making them essential for the development of functional cell walls. Therefore exposure to these fungicides results in abnormal growth and eventually death of sensitive fungi. Squalene-epoxidase inhibitor fungicides include thiocarbamate and allylamine fungicides. The thiocarbamates include pyributicarb. The allylamines include naftifine and terbinafine.

(19) “Polyoxin fungicides” (Fungicide Resistance Action Committee (FRAC) code 19) inhibit chitin synthase. Examples include polyoxin.

(20) “Phenylurea fungicides” (Fungicide Resistance Action Committee (FRAC) code 20) are proposed to affect cell division. Examples include pencycuron.

(21) “Quinone inside inhibitor (QiI) fungicides” (Fungicide Resistance Action Committee (FRAC) code 21) inhibit Complex III mitochondrial respiration in fungi by affecting ubiquinol reductase. Reduction of ubiquinol is blocked at the “quinone inside” (Qᵢ) site of the cytochrome bc₁ complex, which is located in the inner mitochondrial membrane of fungi. Inhibiting mitochondrial respiration prevents normal fungal growth and development. Quinone inside inhibitor fungicides include cyanoimidazole and sulfamoyltriazole fungicides. The cyanoimidazoles include cyazofamid. The sulfamoyltriazoles include amisulbrom.

(22) “Benzamide fungicides” (Fungicide Resistance Action Committee (FRAC) code 22) inhibit mitosis by binding to β-tubulin and disrupting microtubule assembly Inhibition of microtubule assembly can disrupt cell division, transport within the cell and cell structure. Examples include zoxamide.

(23) “Enopyranuronic acid antibiotic fungicides” (Fungicide Resistance Action Committee (FRAC) code 23) inhibit growth of fungi by affecting protein biosynthesis. Examples include blasticidin-S.

(24) “Hexopyranosyl antibiotic fungicides” (Fungicide Resistance Action Committee (FRAC) code 24) inhibit growth of fungi by affecting protein biosynthesis. Examples include kasugamycin.

(25) “Glucopyranosyl antibiotic: protein synthesis fungicides” (Fungicide Resistance Action Committee (FRAC) code 25) inhibit growth of fungi by affecting protein biosynthesis. Examples include streptomycin.

(26) “Glucopyranosyl antibiotic: trehalase and inositol biosynthesis fungicides” (Fungicide Resistance Action Committee (FRAC) code 26) inhibit trehalase in inositol biosynthesis pathway. Examples include validamycin.

(27) “Cyanoacetamideoxime fungicides (Fungicide Resistance Action Committee (FRAC) code 27) include cymoxanil.

(28) “Carbamate fungicides” (Fungicide Resistance Action Committee (FRAC) code 28) are considered multi-site inhibitors of fungal growth. They are proposed to interfere with the synthesis of fatty acids in cell membranes, which then disrupts cell membrane permeability. Propamacarb, propamacarb-hydrochloride, iodocarb, and prothiocarb are examples of this fungicide class.

(29) “Oxidative phosphorylation uncoupling fungicides” (Fungicide Resistance Action Committee (FRAC) code 29) inhibit fungal respiration by uncoupling oxidative phosphorylation. Inhibiting respiration prevents normal fungal growth and development. This class includes 2,6-dinitroanilines such as fluazinam, pyrimidonehydrazones such as ferimzone and dinitrophenyl crotonates such as dinocap, meptyldinocap and binapacryl.

(30) “Organo tin fungicides” (Fungicide Resistance Action Committee (FRAC) code 30) inhibit adenosine triphosphate (ATP) synthase in oxidative phosphorylation pathway. Examples include fentin acetate, fentin chloride and fentin hydroxide.

(31) “Carboxylic acid fungicides” (Fungicide Resistance Action Committee (FRAC) code 31) inhibit growth of fungi by affecting deoxyribonucleic acid (DNA) topoisomerase type II (gyrase). Examples include oxolinic acid.

(32) “Heteroaromatic fungicides” (Fungicide Resistance Action Committee (FRAC) code 32) are proposed to affect DNA/ribonucleic acid (RNA) synthesis. Heteroaromatic fungicides include isoxazole and isothiazolone fungicides. The isoxazoles include hymexazole and the isothiazolones include octhilinone.

(33) “Phosphonate fungicides” (Fungicide Resistance Action Committee (FRAC) code 33) include phosphorous acid and its various salts, including fosetyl-aluminum.

(34) “Phthalamic acid fungicides” (Fungicide Resistance Action Committee (FRAC) code 34) include teclofthalam.

(35) “Benzotriazine fungicides” (Fungicide Resistance Action Committee (FRAC) code 35) include triazoxide.

(36) “Benzene-sulfonamide fungicides” (Fungicide Resistance Action Committee (FRAC) code 36) include flusulfamide.

(37) “Pyridazinone fungicides” (Fungicide Resistance Action Committee (FRAC) code 37) include diclomezine.

(38) “Thiophene-carboxamide fungicides” (Fungicide Resistance Action Committee (FRAC) code 38) are proposed to affect ATP production. Examples include silthiofam.

(39) “Pyrimidinamide fungicides” (Fungicide Resistance Action Committee (FRAC) code 39) inhibit growth of fungi by affecting phospholipid biosynthesis and include diflumetorim.

(40) “Carboxylic acid amide (CAA) fungicides” (Fungicide Resistance Action Committee (FRAC) code 40) are proposed to inhibit phospholipid biosynthesis and cell wall deposition. Inhibition of these processes prevents growth and leads to death of the target fungus. Carboxylic acid amide fungicides include cinnamic acid amide, valinamide carbamate and mandelic acid amide fungicides. The cinnamic acid amides include dimethomorph and flumorph. The valinamide carbamates include benthiavalicarb, benthiavalicarb-isopropyl, iprovalicarb and valiphenal. The mandelic acid amides include mandipropamid, N-[2-[4-[[3-(4-chlorophenyl)-2-propyn-1-yl]oxy]-3-methoxyphenyl]ethyl]-3-methyl-2-[(methylsulfonyl)amino]butanamide and N-[2-[4-[[3-(4-chlorophenyl)-2-propyn-1-yl]oxy]-3-methoxyphenyl]ethyl]-3-methyl-2-[(ethylsulfonyl)amino]butanamide.

(41) “Tetracycline antibiotic fungicides” (Fungicide Resistance Action Committee (FRAC) code 41) inhibit growth of fungi by affecting complex 1 nicotinamide adenine dinucleotide (NADH) oxidoreductase. Examples include oxytetracycline.

(42) “Thiocarbamate fungicides (b42)” (Fungicide Resistance Action Committee (FRAC) code 42) include methasulfocarb.

(43) “Benzamide fungicides” (Fungicide Resistance Action Committee (FRAC) code 43) inhibit growth of fungi by delocalization of spectrin-like proteins. Examples include acylpicolide fungicides such as fluopicolide and fluopyram.

(44) “Host plant defense induction fungicides” (Fungicide Resistance Action Committee (FRAC) code P) induce host plant defense mechanisms. Host plant defense induction fungicides include benzo-thiadiazole, benzisothiazole and thiadiazole-carboxamide fungicides. The benzo-thiadiazoles include acibenzolar-5-methyl. The benzisothiazoles include probenazole. The thiadiazole-carboxamides include tiadinil and isotianil.

(45) “Multi-site contact fungicides” inhibit fungal growth through multiple sites of action and have contact/preventive activity. This class of fungicides includes: (45.1) “copper fungicides” (Fungicide Resistance Action Committee (FRAC) code M1)”, (45.2) “sulfur fungicides” (Fungicide Resistance Action Committee (FRAC) code M2), (45.3) “dithiocarbamate fungicides” (Fungicide Resistance Action Committee (FRAC) code M3), (45.4) “phthalimide fungicides” (Fungicide Resistance Action Committee (FRAC) code M4), (45.5) “chloronitrile fungicides” (Fungicide Resistance Action Committee (FRAC) code M5), (45.6) “sulfamide fungicides” (Fungicide Resistance Action Committee (FRAC) code M6), (45.7) “guanidine fungicides” (Fungicide Resistance Action Committee (FRAC) code M7), (45.8) “triazine fungicides” (Fungicide Resistance Action Committee (FRAC) code M8) and (45.9) “quinone fungicides” (Fungicide Resistance Action Committee (FRAC) code M9). “Copper fungicides” are inorganic compounds containing copper, typically in the copper(II) oxidation state; examples include copper oxychloride, copper sulfate and copper hydroxide, including compositions such as Bordeaux mixture (tribasic copper sulfate). “Sulfur fungicides” are inorganic chemicals containing rings or chains of sulfur atoms; examples include elemental sulfur. “Dithiocarbamate fungicides” contain a dithiocarbamate molecular moiety; examples include mancozeb, metiram, propineb, ferbam, maneb, thiram, zineb and ziram. “Phthalimide fungicides” contain a phthalimide molecular moiety; examples include folpet, captan and captafol. “Chloronitrile fungicides” contain an aromatic ring substituted with chloro and cyano; examples include chlorothalonil. “Sulfamide fungicides” include dichlofluanid and tolyfluanid. “Guanidine fungicides” include dodine, guazatine, iminoctadine albesilate and iminoctadine triacetate. “Triazine fungicides” include anilazine. “Quinone fungicides” include dithianon.

(46) “Fungicides other than fungicides of classes (1) through (45)” include certain fungicides whose mode of action may be unknown. These include: (46.1) “thiazole carboxamide fungicides” (Fungicide Resistance Action Committee (FRAC) code U5), (46.2) “phenyl-acetamide fungicides” (Fungicide Resistance Action Committee (FRAC) code U6), (46.3) “quinazolinone fungicides” (Fungicide Resistance Action Committee (FRAC) code U7) and (46.4) “benzophenone fungicides” (Fungicide Resistance Action Committee (FRAC) code U8). The thiazole carboxamides include ethaboxam. The phenyl-acetamides include cyflufenamid and N-[[(cyclopropylmethoxy)amino][6-(difluoromethoxy)-2,3-difluorophenyl]-methylene]henzeneacetamide. The quinazolinones include proquinazid and 2-butoxy-6-iodo-3-propyl-4H-1-benzopyran-4-one. The benzophenones include metrafenone. The (b46) class also includes bethoxazin, neo-asozin (ferric methanearsonate), pyrroInitrin, quinomethionate, N-[2-[4-[[3-(4-chlorophenyl)-2-propyn-1-yl]oxy]-3-methoxy-phenyl]ethyl]-3-methyl-2-[(methylsulfonyl)amino]butanamide, N-[2-[4-[[3-(4-chloro-phenyl)-2-propyn-1-yl]oxy]-3-methoxyphenyl]ethyl]-3-methyl-2-[(ethylsulfonyl)amino]-butanamide, 2-[[2-fluoro-5-(trifluoromethyl)phenyl]thio]-2-[3-(2-methoxyphenyl)-2-thiazo-lidinylidene]acetonitrile, 3-[5-(4-chlorophenyl)-2,3-dimethyl-3-isoxazolidinyl]pyridine, 4-fluorophenyl N-[1-[[[1-(4-cyanophenyl)ethyl]sulfonyl]methyl]propyl]carbamate, 5-chloro-6-(2,4,6-trifluorophenyl)-7-(4-methylpiperidin-1-yl) [1,2,4]triazolo[1,5-c]pyrimidine, N-(4-chloro-2-nitrophenyl)-N-ethyl-4-methylbenzenesulfonamide, N-[[(cyclopropylmethoxy)-amino][6-(difluoromethoxy)-2,3-difluorophenyl]methylene] benzeneacetamide, N-[4-[4-chloro-3-(trifluoromethyl)phenoxy]-2,5-dimethylphenyl]-N-ethyl-N-methylmethanimid-amide and 1-[(2-propenylthio)carbonyl]-2-(1-methylethyl)-4-(2-methylphenyl)-5-amino-1H-pyrazol-3-one.

Therefore of note is a mixture (i.e. composition) comprising a compound of Formula 1 and at least one fungicidal compound selected from the group consisting of the aforedescribed classes (1) through (46). Also of note is a composition comprising said mixture (in fungicidally effective amount) and further comprising at least one additional component selected from the group consisting of surfactants, solid diluents and liquid diluents. Of particular note is a mixture (i.e. composition) comprising a compound of Formula 1 and at least one fungicidal compound selected from the group of specific compounds listed above in connection with classes (1) through (46). Also of particular note is a composition comprising said mixture (in fungicidally effective amount) and further comprising at least one additional surfactant selected from the group consisting of surfactants, solid diluents and liquid diluents.

Examples of other biologically active compounds or agents with which compounds of this invention can be formulated are: insecticides such as abamectin, acephate, acequinocyl, acetamiprid, acrinathrin, amidoflumet, amitraz, avermectin, azadirachtin, azinphos-methyl, bifenthrin, bifenazate, bistrifluoron, borate, 3-bromo-1-(3-chloro-2-pyridinyl)-N-[4-cyano-2-methyl-6-[(methylamino)carbonyl]phenyl]-1H-pyrazole-5-carboxamide, buprofezin, cadusafos, carbaryl, carbofuran, cartap, carzol, chlorantraniliprole, chlorfenapyr, chlorfluazuron, chlorpyrifos, chlorpyrifos-methyl, chromafenozide, clofentezin, clothianidin, cyflumetofen, cyfluthrin, beta-cyfluthrin, cyhalothrin, gamma-cyhalothrin, lambda-cyhalothrin, cypermethrin, alpha-cypermethrin, zeta-cypermethrin, cyromazine, deltamethrin, diafenthiuron, diazinon, dieldrin, diflubenzuron, dimefluthrin, dimehypo, dimethoate, dinotefuran, diofenolan, emamectin, endosulfan, esfenvalerate, ethiprole, etofenprox, etoxazole, fenbutatin oxide, fenothiocarb, fenoxycarb, fenpropathrin, fenvalerate, fipronil, flonicamid, flubendiamide, flucythrinate, flufenerim, flufenoxuron, fluvalinate, tau-fluvalinate, fonophos, formetanate, fosthiazate, halofenozide, hexaflumuron, hexythiazox, hydramethylnon, imidacloprid, indoxacarb, insecticidal soaps, isofenphos, lufenuron, malathion, metaflumizone, metaldehyde, methamidophos, methidathion, methiodicarb, methomyl, methoprene, methoxychlor, metofluthrin, monocrotophos, methoxyfenozide, nitenpyram, nithiazine, novaluron, noviflumuron, oxamyl, parathion, parathion-methyl, permethrin, phorate, phosalone, phosmet, phosphamidon, pirimicarb, profenofos, profluthrin, propargite, protrifenbute, pymetrozine, pyrafluprole, pyrethrin, pyridaben, pyridalyl, pyrifluquinazon, pyriprole, pyriproxyfen, rotenone, ryanodine, spinetoram, spinosad, spirodiclofen, spiromesifen, spirotetramat, sulprofos, tebufenozide, tebufenpyrad, teflubenzuron, tefluthrin, terbufos, tetrachlorvinphos, tetramethrin, thiacloprid, thiamethoxam, thiodicarb, thiosultap-sodium, tolfenpyrad, tralomethrin, triazamate, trichlorfon, triflumuron, Bacillus thuringiensis delta-endotoxins, entomopathogenic bacteria, entomopathogenic viruse

Compounds of this invention and compositions thereof can be applied to plants genetically transformed to express proteins toxic to invertebrate pests (such as Bacillus thuringiensis delta-endotoxins). The effect of the exogenously applied fungicidal compounds of this invention may be synergistic with the expressed toxin proteins.

General references for agricultural protectants (i.e. insecticides, fungicides, nematocides, acaricides, herbicides and biological agents) include The Pesticide Manual, 13th Edition, C. D. S. Tomlin, Ed., British Crop Protection Council, Farnham, Surrey, U.K., 2003 and The BioPesticide Manual, 2nd Edition, L. G. Copping, Ed., British Crop Protection Council, Farnham, Surrey, U.K., 2001.

For embodiments where one or more of these various mixing partners are used, the weight ratio of these various mixing partners (in total) to the compound of Formula 1 is typically between about 1:3000 and about 3000:1. Of note are weight ratios between about 1:300 and about 300:1 (for example ratios between about 1:30 and about 30:1). One skilled in the art can easily determine through simple experimentation the biologically effective amounts of active ingredients necessary for the desired spectrum of biological activity. It will be evident that including these additional components may expand the spectrum of diseases controlled beyond the spectrum controlled by the compound of Formula 1 alone.

In certain instances, combinations of a compound of this invention with other biologically active (particularly fungicidal) compounds or agents (i.e. active ingredients) can result in a greater-than-additive (i.e. synergistic) effect. Reducing the quantity of active ingredients released in the environment while ensuring effective pest control is always desirable. When synergism of fungicidal active ingredients occurs at application rates giving agronomically satisfactory levels of fungal control, such combinations can be advantageous for reducing crop production cost and decreasing environmental load.

Of note is a combination of a compound of Formula 1 with at least one other fungicidal active ingredient. Of particular note is such a combination where the other fungicidal active ingredient has different site of action from the compound of Formula 1. In certain instances, a combination with at least one other fungicidal active ingredient having a similar spectrum of control but a different site of action will be particularly advantageous for resistance management. Thus, a composition of the present invention can further comprise a biologically effective amount of at least one additional fungicidal active ingredient having a similar spectrum of control but a different site of action.

Of particular note are compositions which in addition to compound of Formula 1 include at least one compound selected from the group consisting of (1) alkylenebis(dithiocarbamate) fungicides; (2) cymoxanil; (3) phenylamide fungicides; (4) pyrimidinone fungicides; (5) chlorothalonil; (6) carboxamides acting at complex II of the fungal mitochondrial respiratory electron transfer site; (7) quinoxyfen; (8) metrafenone; (9) cyflufenamid; (10) cyprodinil; (11) copper compounds; (12) phthalimide fungicides; (13) fosetyl-aluminum; (14) benzimidazole fungicides; (15) cyazofamid; (16) fluazinam; (17) iprovalicarb; (18) propamocarb; (19) validomycin; (20) dichlorophenyl dicarboximide fungicides; (21) zoxamide; (22) fluopicolide; (23) mandipropamid; (24) carboxylic acid amides acting on phospholipid biosynthesis and cell wall deposition; (25) dimethomorph; (26) non-DMI sterol biosynthesis inhibitors; (27) inhibitors of demethylase in sterol biosynthesis; (28) bc₁ complex fungicides; and salts of compounds of (1) through (28).

Further descriptions of classes of fungicidal compounds are provided below.

Pyrimidinone fungicides (group (4)) include compounds of Formula A1

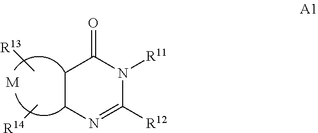

wherein M forms a fused phenyl, thiophene or pyridine ring; R¹¹ is C₁-C₆ alkyl; R¹² is C₁-C₆ alkyl or C₁-C₆ alkoxy; R¹³ is halogen; and R¹⁴ is hydrogen or halogen.

Pyrimidinone fungicides are described in PCT Patent Application Publication WO 94/26722 and U.S. Pat. Nos. 6,066,638, 6,245,770, 6,262,058 and 6,277,858. Of note are pyrimidinone fungicides selected from the group: 6-bromo-3-propyl-2-propyloxy-4(3H)-quinazolinone, 6,8-diiodo-3-propyl-2-propyloxy-4(3H)-quinazolinone, 6-iodo-3-propyl-2-propyloxy-4(3H)-quinazolinone (proquinazid), 6-chloro-2-propoxy-3-propyl-thieno[2,3-d]pyrimidin-4(3H)-one, 6-bromo-2-propoxy-3-propylthieno[2,3-d]pyrimidin-4(3H)-one, 7-bromo-2-propoxy-3-propylthieno[3,2-d]pyrimidin-4(3H)-one, 6-bromo-2-propoxy-3-propylpyrido[2,3-d]pyrimidin-4(3H)-one, 6,7-dibromo-2-propoxy-3-propyl-thieno[3,2-d]pyrimidin-4(3H)-one, and 3-(cyclopropylmethyl)-6-iodo-2-(propylthio)pyrido-[2,3-d]pyrimidin-4(3H)-one.

Sterol biosynthesis inhibitors (group (27)) control fungi by inhibiting enzymes in the sterol biosynthesis pathway. Demethylase-inhibiting fungicides have a common site of action within the fungal sterol biosynthesis pathway, involving inhibition of demethylation at position 14 of lanosterol or 24-methylene dihydrolanosterol, which are precursors to sterols in fungi. Compounds acting at this site are often referred to as demethylase inhibitors, DMI fungicides, or DMIs. The demethylase enzyme is sometimes referred to by other names in the biochemical literature, including cytochrome P-450 (14DM). The demethylase enzyme is described in, for example, J. Biol. Chem. 1992, 267, 13175-79 and references cited therein. DMI fungicides are divided between several chemical classes: azoles (including triazoles and imidazoles), pyrimidines, piperazines and pyridines. The triazoles include azaconazole, bromuconazole, cyproconazole, difenoconazole, diniconazole (including diniconazole-M), epoxiconazole, etaconazole, fenbuconazole, fluquinconazole, flusilazole, flutriafol, hexaconazole, imibenconazole, ipconazole, metconazole, myclobutanil, penconazole, propiconazole, prothioconazole, quinconazole, simeconazole, tebuconazole, tetraconazole, triadimefon, triadimenol, triticonazole and uniconazole. The imidazoles include clotrimazole, econazole, imazalil, isoconazole, miconazole, oxpoconazole, prochloraz and triflumizole. The pyrimidines include fenarimol, nuarimol and triarimol. The piperazines include triforine. The pyridines include buthiobate and pyrifenox. Biochemical investigations have shown that all of the above mentioned fungicides are DMI fungicides as described by K. H. Kuck et al. in Modern Selective Fungicides—Properties, Applications and Mechanisms of Action, H. Lyr (Ed.), Gustav Fischer Verlag: New York, 1995, 205-258.

bc₁ Complex Fungicides (group 28) have a fungicidal mode of action which inhibits the bc₁ complex in the mitochondrial respiration chain. The bc₁ complex is sometimes referred to by other names in the biochemical literature, including complex III of the electron transfer chain, and ubihydroquinone:cytochrome c oxidoreductase. This complex is uniquely identified by Enzyme Commission number EC1.10.2.2. The bc₁ complex is described in, for example, J. Biol. Chem. 1989, 264, 14543-48; Methods Enzymol. 1986, 126, 253-71; and references cited therein. Strobilurin fungicides such as azoxystrobin, dimoxystrobin, enestroburin (SYP-Z071), fluoxastrobin, kresoxim-methyl, metominostrobin, orysastrobin, picoxystrobin, pyraclostrobin and trifloxystrobin are known to have this mode of action (H. Sauter et al., Angew. Chem. Int. Ed. 1999, 38, 1328-1349). Other fungicidal compounds that inhibit the bc₁ complex in the mitochondrial respiration chain include famoxadone and fenamidone.

Alkylenebis(dithiocarbamate)s (group (1)) include compounds such as mancozeb, maneb, propineb and zineb. Phenylamides (group (3)) include compounds such as metalaxyl, benalaxyl, furalaxyl and oxadixyl. Carboxamides (group (6)) include compounds such as boscalid, carboxin, fenfuram, flutolanil, furametpyr, mepronil, oxycarboxin, thifluzamide, penthiopyrad and N-[2-(1,3-dimethylbutyl)phenyl]-5-fluoro-1,3-dimethyl-1H-pyrazole-4-carboxamide (PCT Patent Publication WO 2003/010149), and are known to inhibit mitochondrial function by disrupting complex II (succinate dehydrogenase) in the respiratory electron transport chain. Copper compounds (group (11)) include compounds such as copper oxychloride, copper sulfate and copper hydroxide, including compositions such as Bordeaux mixture (tribasic copper sulfate). Phthalimides (group (12)) include compounds such as folpet and captan. Benzimidazole fungicides (group (14)) include benomyl and carbendazim. Dichlorophenyl dicarboximide fungicides (group (20)) include chlozolinate, dichlozoline, iprodione, isovaledione, myclozolin, procymidone and vinclozolin.

Non-DMI sterol biosynthesis inhibitors (group (26)) include morpholine and piperidine fungicides. The morpholines and piperidines are sterol biosynthesis inhibitors that have been shown to inhibit steps in the sterol biosynthesis pathway at a point later than the inhibitions achieved by the DMI sterol biosynthesis (group (27)). The morpholines include aldimorph, dodemorph, fenpropimorph, tridemorph and trimorphamide. The piperidines include fenpropidin

Of further note are combinations of compounds of Formula 1 with azoxystrobin, kresoxim-methyl, trifloxystrobin, pyraclostrobin, picoxystrobin, dimoxystrobin, metominostrobin/fenominostrobin, carbendazim, chlorothalonil, quinoxyfen, metrafenone, cyflufenamid, fenpropidine, fenpropimorph, bromuconazole, cyproconazole, difenoconazole, epoxiconazole, fenbuconazole, flusilazole, hexaconazole, ipconazole, metconazole, penconazole, propiconazole, proquinazid, prothioconazole, tebuconazole, triticonazole, famoxadone, prochloraz, penthiopyrad and boscalid (nicobifen).

Preferred for better control of plant diseases caused by fungal plant pathogens (e.g., lower use rate or broader spectrum of plant pathogens controlled) or resistance management are mixtures of a compound of this invention with a fungicide selected from the group: azoxystrobin, kresoxim-methyl, trifloxystrobin, pyraclostrobin, picoxystrobin, dimoxystrobin, metominostrobin/fenominostrobin, quinoxyfen, metrafenone, cyflufenamid, fenpropidine, fenpropimorph, cyproconazole, epoxiconazole, flusilazole, metconazole, propiconazole, proquinazid, prothioconazole, tebuconazole, triticonazole, famoxadone and penthiopyrad.

Specifically preferred mixtures (compound numbers refer to compounds in Index Tables A-B) are selected from the group: combinations of Compound 72, Compound 89, Compound 110, Compound 121, Compound 124, Compound 133, Compound 135 or Compound 137 with azoxystrobin, combinations of Compound 72, Compound 89, Compound 110, Compound 121, Compound 124, Compound 133, Compound 135 or Compound 137 with kresoxim-methyl, combinations of Compound 72, Compound 89, Compound 110, Compound 121, Compound 124, Compound 133, Compound 135 or Compound 137 with trifloxystrobin, combinations of Compound 72, Compound 89, Compound 110, Compound 121, Compound 124, Compound 133, Compound 135 or Compound 137 with pyraclostrobin, combinations of Compound 72, Compound 89, Compound 110, Compound 121, Compound 124, Compound 133, Compound 135 or Compound 137 with picoxystrobin, combinations of Compound 72, Compound 89, Compound 110, Compound 121, Compound 124, Compound 133, Compound 135 or Compound 137 with dimoxystrobin, combinations of Compound 72, Compound 89, Compound 110, Compound 121, Compound 124, Compound 133, Compound 135 or Compound 137 with metominostrobin/fenominostrobin, combinations of Compound 72, Compound 89, Compound 110, Compound 121, Compound 124, Compound 133, Compound 135 or Compound 137 with quinoxyfen, combinations of Compound 72, Compound 89, Compound 110, Compound 121, Compound 124, Compound 133, Compound 135 or Compound 137 with metrafenone, combinations of Compound 72, Compound 89, Compound 110, Compound 121, Compound 124, Compound 133, Compound 135 or Compound 137 with cyflufenamid, combinations of Compound 72, Compound 89, Compound 110, Compound 121, Compound 124, Compound 133, Compound 135 or Compound 137 with fenpropidine, combinations of Compound 72, Compound 89, Compound 110, Compound 121, Compound 124, Compound 133, Compound 135 or Compound 137 with fenpropimorph, combinations of Compound 72, Compound 89, Compound 110, Compound 121, Compound 124, Compound 133, Compound 135 or Compound 137 with cyproconazole, combinations of Compound 72, Compound 89, Compound 110, Compound 121, Compound 124, Compound 133, Compound 135 or Compound 137 with epoxiconazole, combinations of Compound 72, Compound 89, Compound 110, Compound 121, Compound 124, Compound 133, Compound 135 or Compound 137 with flusilazole, combinations of Compound 72, Compound 89, Compound 110, Compound 121, Compound 124, Compound 133, Compound 135 or Compound 137 with metconazole, combinations of Compound 72, Compound 89, Compound 110, Compound 121, Compound 124, Compound 133, Compound 135 or Compound 137 with propiconazole, combinations of Compound 72, Compound 89, Compound 110, Compound 121, Compound 124, Compound 133, Compound 135 or Compound 137 with proquinazid, combinations of Compound 72, Compound 89, Compound 110, Compound 121, Compound 124, Compound 133, Compound 135 or Compound 137 with prothioconazole, combinations of Compound 72, Compound 89, Compound 110, Compound 121, Compound 124, Compound 133, Compound 135 or Compound 137 with tebuconazole, combinations of Compound 72, Compound 89, Compound 110, Compound 121, Compound 124, Compound 133, Compound 135 or Compound 137 with triticonazole, combinations of Compound 72, Compound 89, Compound 110, Compound 121, Compound 124, Compound 133, Compound 135 or Compound 137 with famoxadone, and combinations of Compound 72, Compound 89, Compound 110, Compound 121, Compound 124, Compound 133, Compound 135 or Compound 137 with penthiopyrad.

The following tests demonstrate the control efficacy of compounds of this invention on specific pathogens. The pathogen control protection afforded by the compounds is not limited, however, to these species. See Index Tables A-B for compound descriptions.

The following abbreviations are used in the Index Tables which follow: i is iso, c is cyclo, Me is methyl, Et is ethyl, Pr is propyl, i-Pr is isopropyl, Bu is butyl, c-Pr is cyclopropyl, t-Bu is tert-butyl, Ac is acetyl (i.e. C(═O)Me) and Ph is phenyl. The abbreviation “Cmpd.” stands for “Compound”. The abbreviation “Ex.” stands for “Example” and is followed by a number indicating in which example the compound is prepared. In Index Tables A and B the numerical value reported in the column “AP⁺ (M+1)”, is the molecular weight of the observed molecular ion formed by addition of H⁺ (molecular weight of 1) to the molecule having the greatest isotopic abundance (i.e. M). The presence of molecular ions containing one or more higher atomic weight isotopes of lower abundance (e.g., ³⁷Cl, ⁸¹Br) is not reported. The reported M+1 peaks were observed by mass spectrometry using atmospheric pressure chemical ionization (AP⁺).

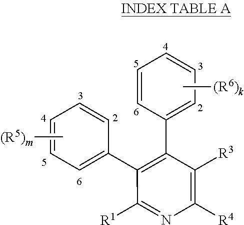

A dash (“-”) in the (R⁵)ₘ column indicates m is 0 and hydrogen is present at all positions.

| | | | | | | m.p. | AP+ |
|---|---|---|---|---|---|---|---|
| Cmpd. | R¹ | R⁴ | R³ | (R⁶)ₖ | (R⁵)ₘ | (° C.) | (M + 1) |
| 1 | Me | H | CO₂Et | 3,5-di-OMe | — | | 378 |
| 2 | Me | H | C(═O)Me | 3,5-di-OMe | — | | 348 |
| 3 | Me | H | C(OH)Me₂ | 3,5-di-OMe | — | | 364 |
| 4 | Me | H | i-Pr | 3,5-di-OMe | — | | 348 |
| 6 | Me | H | CO₂Et | 3,4,5-tri-OMe | — | | 408 |
| 7 | Me | H | CO₂Et | 3-OMe, 6-Cl | — | | 382 |
| 8 | Me | H | CO₂Et | 3,5-di-F | — | | 354 |
| 9 | Me | H | C(═O)NHMe | 3,5-di-OMe | — | | 363 |
| 10 | Me | H | C(═O)NMe₂ | 3,5-di-OMe | — | | 377 |
| 11 | Me | H | CO₂Et | 3,5-di-OMe | 4-F | ** | 396 |
| (Ex. 1) | | | | | | | |
| 12 | Me | H | C(═O)Me | 3,5-di-OMe | 4-F | ** | 366 |
| (Ex. 2) | | | | | | | |
| 13 | Me | H | C(OH)Me₂ | 3,5-di-OMe | 4-F | ** | 382 |
| (Ex. 2) | | | | | | | |
| 14 | Me | H | CCH₃(OH)CH₂CH₃ | 3,5-di-OMe | 4-F | | 396 |
| 15 | Me | H | CO₂Et | 3-OMe, 6-F | — | | 366 |
| 16 | Me | H | C(═CH₂)Me | 3,4,5-tri-OMe | 4-F | ** | 364 |
| (Ex. 3) | | | | | | | |
| 17 | Me | H | C(═O)Me | 3,5-di-OMe | 2-F | | 366 |
| 18 | Me | H | C(OH)Me₂ | 3,5-di-OMe | 2-F | | 382 |
| 19 | Me | H | i-Pr | 3,5-di-OMe | 4-F | ** | 366 |
| (Ex. 4) | | | | | | | |
| 20 | Me | H | CO₂Et | 3,5-di-OMe | 2-F | | 396 |
| 21 | Me | H | i-Pr | 3,5-di-OMe | 2-F | | 366 |
| 22 | Me | Me | Ph | 3,5-di-OMe | 4-F | | 414 |
| 23 | Me | Me | Ph | 3,5-di-OMe | 2-F | | 396 |
| 25 | Me | H | CO₂Et | 3,5-di-OMe, 6-Cl | — | | 412 |
| 26 | Me | H | CO₂Et | 3,5-di-OMe, 6-F | — | | 396 |
| 27 | Et | H | C(═O)Me | 3,5-di-OMe | — | | 362 |
| 28 | Et | H | C(OH)Me₂ | 3,5-di-OMe | — | | 378 |
| 29 | Et | H | C(═CH₂)Me | 3,5-di-OMe | — | | 360 |
| 30 | Et | H | i-Pr | 3,5-di-OMe | — | | 362 |
| 31 | Et | H | CO₂Et | 3,5-di-OMe | — | | 392 |
| 32 | Me | H | 2-F-Ph | 3,5-di-OMe | — | | 400 |
| 34 | Me | H | Ph | 3,5-di-OMe | 4-F | ** | 400 |
| (Ex. 5) | | | | | | | |
| 35 | Me | H | 2-F-Ph | 3,5-di-OMe | 4-F | 123-125 | 418 |
| 36 | Me | H | 4-F-Ph | 3,5-di-OMe | — | | 400 |
| 37 | Me | H | CO₂Et | 3,5-di-OMe | 2,6-di-F | | 414 |
| 38 | Me | H | 2,4-di-F-Ph | 3,5-di-OMe | 4-F | | 436 |
| 40 | Me | H | C(═O)Me | 3,5-di-OMe | 2,4,6-tri-F | ** | 402 |
| (Ex. 10) | | | | | | | |
| 41 | Me | H | C(═O)Me | 3,5-di-OMe | 2,6-di-F | | 384 |
| 42 | Me | H | C(OH)Me₂ | 3,5-di-OMe | 2,6-di-F | | 400 |
| 43 | Me | H | C(═CH₂)Me | 3,5-di-OMe | 2,4,6-tri-F | 128-130 | 400 |
| 44 | Me | H | C(OH)Me₂ | 3,5-di-OMe | 2,4,6-tri-F | 166-167 | 418 |
| (Ex. 10) | | | | | | | |
| 45 | Me | H | i-Pr | 3,5-di-OMe | 2,6-di-F | | 384 |
| 46 | Me | H | 1-cyclohexen-1-yl | 3,5-di-OMe | 4-F | 147-150 | 404 |
| 47 | Me | H | c-hexyl | 3,5-di-OMe | 4-F | 142-143 | 406 |
| 48 | Me | H | i-Pr | 3,5-di-OMe | 2,4,6-tri-F | 85-88 | 402 |
| 49 | Me | Cl | c-pentyl | 3,5-di-OMe | 4-F | ** | 426 |
| (Ex. 7) | | | | | | | |
| 50 | Me | H | c-pentyl | 3,5-di-OMe | 4-F | 129-130 | 392 |
| (Ex. 8) | | | | | | | |
| 54 | Me | H | CO₂Et | 3,4-di-OMe | — | | 362 |
| 55 | Me | H | CO₂Et | 1,3-benzodioxol-5-yl | — | | 339 |
| 58 | Me | H | CO₂Et | 4-Me-1-piperidinyl | — | | 378 |
| 59 | Me | H | tetrahydropyran-4-yl | 3,5-di-OMe | 4-F | | 408 |
| 60 | Me | H | CO₂Et | 3,5-di-OMe | 4-OMe | | 408 |
| 61 | Me | H | C(═O)Me | 3,5-di-OMe | 4-OMe | | 378 |
| 62 | Me | H | C(OH)Me₂ | 3,5-di-OMe | 4-OMe | 153-154 | 394 |
| 63 | Me | H | CH(CH₃)CH₂CH₃ | 3,5-di-OMe | 4-F | 119-122 | 380 |
| 64 | Me | H | C(═CH₂)Me | 3,5-di-OMe | 4-OMe | 115-117 | 376 |
| 65 | Me | H | i-Pr | 3,5-di-OMe | 4-OMe | 134-136 | 378 |
| 66 | Me | H | CO₂Et | 3,5-di-OMe | 2,3,6-tri-F | | 432 |
| 67 | Me | H | c-pentyl | 3,5-di-OMe | 2,6-di-F | 141-143 | 410 |
| 68 | Me | H | C(═O)Me | 3,5-di-OMe | 2,3,6-tri-F | | 402 |
| 69 | Me | H | C(OH)Me₂ | 3,5-di-OMe | 2,3,6-tri-F | | 418 |
| 70 | Me | H | CO₂Et | 3,5-di-OMe | 2,6-di-F-4-OMe | | 444 |
| 71 | Me | H | C(═O)Me | 3,5-di-OMe | 2,6-di-F-4-OMe | | 414 |
| 72 | Me | H | C(OH)Me₂ | 3,5-di-OMe | 2,6-di-F-4-OMe | | 430 |
| 73 | Me | H | CH(CH₃)CH₂CH₃ | 3,5-di-OMe | 2,4,6-tri-F | 91-94 | 416 |
| 74 | Me | H | C(═CH₂)Me | 3,5-di-OMe | 2,6-di-F-4-OMe | 130-132 | 412 |
| 75 | Me | H | i-Pr | 3,5-di-OMe | 2,6-di-F-4-OMe | 105-107 | 414 |
| 76 | Cl | H | 2,6-di-F-Ph | 3,5-di-OMe | 2,6-di-F | 179-181 | 474 |
| 77 | Me | H | C(═CH₂)Me | 3,5-di-OMe | 2,3,6-tri-F | 101-106 | 400 |
| 78 | Me | H | i-Pr | 3,5-di-OMe | 2,3,6-tri-F | 138-139 | 402 |
| 79 | Me | H | tetrahydro-2H-4-yl | 3,5-di-OMe | 2,4,6-tri-F | | 444 |
| 80 | Cl | H | 2,4,6-tri-F-Ph | 3,5-di-OMe | 2,4,6-tri-F | 120-122 | 510 |
| 81 | CN | H | 2,6-di-F-Ph | 3,5-di-OMe | 2,6-di-F | | 465 |
| 82 | Me | H | CH(CH₃)CH₂OH | 3,5-di-OMe | 4-OMe | | 394 |
| 83 | Me | H | C(OH)CH₃CH₂OH | 3,5-di-OMe | 4-OMe | | 410 |
| 84 | Me | H | tetrahydro-2H- | 3,5-di-OMe | 4-F | | 425 |
| | | | thiopyran-4-yl | | | | |
| 85 | Me | H | C(OH)CH₃CH₂OH | 3,5-di-OMe | 4-OMe | | 410 |
| 86 | Me | H | Ph | 3,5-di-OMe | 2-F | | 400 |
| 87 | Me | H | Ph | 3,5-di-OMe | 2,6-di-F | | 418 |
| 88 | Me | H | 4-F-Ph | 3,5-di-OMe | 4-F | | 418 |
| 89 | Me | H | 2-F-Ph | 3,5-di-OMe | 2,4,6-tri-F | 123-127 | 454 |
| (Ex. 14) | | | | | | | |
| 90 | Me | H | CH(CH₃)CH₂OH | 3,5-di-OMe | 2,6-di-F | | 400 |
| 91 | Me | H | Ph | 3,5-di-OMe | 2,4-di-F | | 418 |
| 92 | Me | H | CH(CH₃)CH₂OH | 3,5-di-OMe | 2,6-di-F-4-OMe | | 430 |
| 93 | Me | H | 4-F-Ph | 3,5-di-OMe | 2,4,6-tri-F | | 454 |
| 94 | Me | H | 4-F-Ph | 3,5-di-OMe | 2-F | | 418 |
| 95 | Me | H | 4-F-Ph | 3,5-di-OMe | 2,4-di-F | | 436 |
| 96 | Me | H | CH₂OH | 3,5-di-OMe | 2,4,6-tri-F | ** | 390 |
| (Ex. 11) | | | | | | | |
| 97 | Me | H | pyrazol-1-ylmethyl | 3,5-di-OMe | 2,4,6-tri-F | | 440 |
| 98 | Me | H | C(OH)(CH₂CH₃)₂ | 3,5-di-OMe | 2,4,6-tri-F | | 446 |
| 99 | Me | H | CH(OH)CH₂CH₃ | 3,5-di-OMe | 2,4,6-tri-F | | 418 |
| 100 | Me | H | 2-F-Ph | 3,5-di-OH | 4-F | | 390 |
| 101 | Me | H | 2-F-Ph | 3,5-di-OCHF₂ | 4-F | | 490 |
| 102 | Me | H | C(OAc)(CH₃)₂ | 3,5-di-OMe | 2,4,6-tri-F | | 460 |
| 103 | Me | H | CH(OH)CH(CH₃)₂ | 3,5-di-OMe | 2,4,6-tri-F | | 432 |
| 104 | Me | H | [1,2,4]triazol-1- | 3,5-di-OMe | 2,4,6-tri-F | | 441 |
| | | | ylmethyl | | | | |
| 105 | Me | H | C(═O)Me | 3,5-di-OMe | 2,6-di-F-3-OMe | 124-128 | 414 |
| 106 | Me | H | C(OH)(CH₃)₂ | 3,5-di-OMe | 2,6-di-F-3-OMe | 150-155 | 430 |
| 107 | Me | H | CH(OH)CH₃ | 3,5-di-OMe | 2,4,6-tri-F | | 404 |
| 108 | Me | H | 3-CF₃-pyrazol-1- | 3,5-di-OMe | 2,4,6-tri-F | | 508 |
| | | | ylmethyl | | | | |
| 109 | Me | H | CH₂OPh | 3,5-di-OMe | 2,4,6-tri-F | | 466 |
| 110 | Me | H | CH₂Cl | 3,5-di-OMe | 2,4,6-tri-F | 110-115 | 408 |
| 111 | Me | H | piperidin-1-ylmethyl | 3,5-di-OMe | 2,4,6-tri-F | | 457 |
| 112 | Me | H | C(═O)H | 3,5-di-OMe | 2,4,6-tri-F | | 388 |
| 113 | Me | H | C(═O)H | 3,5-di-OMe | 2,6-di-F-3-OMe | | 400 |
| 114 | Me | H | C(═NOH)Me | 3,5-di-OMe | 2,4,6-tri-F | | 417 |
| 115 | Me | H | CH(OH)CH₂CH₃ | 3,5-di-OMe | 2,6-di-F-3-OMe | | 430 |
| 116 | Me | H | CH(OH)CH₃ | 3,5-di-OMe | 2,6-di-F-3-OMe | | 416 |
| 117 | Me | H | C(═NOH)Me | 3,5-di-OMe | 2,4,6-tri-F | | 417 |
| 118 | Me | H | C(═O)Me | 2-Cl-3,5-di-OMe | 2,4,6-tri-F | | 436 |
| 119 | Me | H | C(OH)(CH₃)₂ | 2-Cl-3,5-di-OMe | 2,4,6-tri-F | 190-194 | 452 |
| 120 | Me | H | C(═O)NH₂ | 3,5-di-OMe | 2,4,6-tri-F | | 403 |
| 121 | Me | H | Ph | 3,5-di-OMe | 2,4,6-tri-F | | 436 |
| 122 | Me | H | Br | 3,5-di-OMe | 2,4,6-tri-F | ** | 438 |
| (Ex. 13) | | | | | | | |
| 123 | Me | H | 4-CN-pyrazol-1- | 3,5-di-OMe | 2,4,6-tri-F | | 465 |
| | | | ylmethyl | | | | |
| 124 | Me | H | CH₂CN | 3,5-di-OMe | 2,4,6-tri-F | 131-133 | 399 |
| (Ex. 12) | | | | | | | |
| 125 | Me | H | CH(CH₃)CN | 3,5-di-OMe | 2,4,6-tri-F | 144-145 | 413 |
| 126 | Me | H | 2-F-Ph | 2-Cl-3,5-di-OMe | 4-F | 195-200 | 452 |
| 127 | Me | H | 6-Cl-pyridin-3-yl | 3,5-di-OMe | 2,4,6-tri-F | | 471 |
| 128 | Me | H | pyridine-3-yl | 3,5-di-OMe | 2,4,6-tri-F | 148-150 | 437 |
| 129 | Me | H | Me | 3,5-di-OMe | 2,4,6-tri-F | 132-134 | 374 |
| 130 | Me | H | CO₂Et | 3,5-di-OMe | 2,4,6-tri-F | 93-95 | 432 |
| (Ex. 9) | | | | | | | |
| 131 | Me | H | 2,4-di-OMe- | 3,5-di-OMe | 2,4,6-tri-F | 136-139 | 498 |
| | | | pyrimidin-5-yl | | | | |
| 132 | Me | H | thiophen-5-yl | 3,5-di-OMe | 2,4,6-tri-F | | 442 |
| 133 | Me | H | CH₂CN | 2-Cl-3,5-di-OMe | 2,4,6-tri-F | 159-165 | 433 |
| 134 | Me | H | CH₂Cl | 2-Cl-3,5-di-OMe | 2,4,6-tri-F | 135-137 | 442 |
| 135 | Me | H | 2-F-Ph | 2-Cl-3,5-di-OMe | 2,4,6-tri-F | | 488 |
| 136 | Me | H | 2-F-Ph | 2,6-di-Cl-3,5-di-OMe | 2,4,6-tri-F | | 522 |
| 137 | Me | H | Et | 3,5-di-OMe | 2,4,6-tri-F | 102-105 | 388 |
| 138 | Me | H | ethynyl | 3,5-di-OMe | 2,4,6-tri-F | 129-133 | 384 |
| 140 | Me | H | CH₂Ph | 3,5-di-OMe | 2,4,6-tri-F | 113-116 | 450 |
| 141 | Me | OH | Cl | 2,6-di-F | 4-Cl | 303-305 | |
| 142 | Me | H | Cl | 2,6-di F | 4-Cl | 110-112 | |
| 143 | Me | CN | Me | 4-Cl | 2,6-di-F | 158-160 | |
| 144 | Me | H | Me | 4-Cl | 2,6-di-F | 111-113 | |

**See synthesis example for ¹H NMR data.

INDEX TABLE B

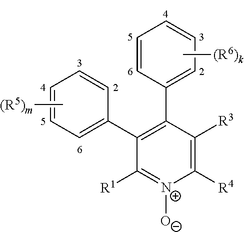

| | | | | | | m.p. | AP+ |
|---|---|---|---|---|---|---|---|
| Cmpd. | R¹ | R⁴ | R³ | (R⁶)ₖ | (R⁵)ₘ | (° C.) | (M + 1) |
| 5 | Me | H | i-Pr | 3,5-di-OMe | — | | 364 |
| 24 | Me | Me | Ph | 3,5-di-OMe | 2-F | | 430 |
| 33 | Me | H | 2-F—Ph | 3,5-di-OMe | — | | 416 |
| 39 | Me | H | 2-F—Ph | 3,5-di-OMe | 4-F | ** | 434 |
| (Ex. 6) | | | | | | | |
| 51 | Me | H | CO₂Et | 3,5-di-OMe | — | | 394 |
| 52 | Me | H | C(OH)Me2 | 3,5-di-OMe | — | | 380 |
| 139 | Me | H | Et | 3,5-di-OMe | 2,4,6-tri-F | 113- | 404 |
| | | | | | | 116 | |

A dash (“—”) in the (R⁵)m column indicates m is 0 and hydrogen is present at all positions.
**See synthesis example for ¹H NMR data.

### Biological Examples of the Invention

General protocol for preparing test suspensions for Tests A-H: The test compounds were first dissolved in acetone in an amount equal to 3% of the final volume and then suspended at the desired concentration (in ppm) in acetone and purified water (50/50 mix) containing 250 ppm of the surfactant Trem® 014 (polyhydric alcohol esters). The resulting test suspensions were then used in tests A-H. Spraying a 200 ppm test suspension to the point of run-off on the test plants was the equivalent of a rate of 500 g/ha.

### Test A

The test suspension was sprayed to the point of run-off on wheat seedlings. The following day the seedlings were inoculated with a spore dust of Erysiphe graminis f. sp. tritici (the causal agent of wheat powdery mildew) and incubated in a growth chamber at 20° C. for 8 days, after which time disease ratings were visually made.

### Test B

The test suspension was sprayed to the point of run-off on wheat seedlings. The following day the seedlings were inoculated with a spore suspension of Puccinia recondite f. sp. tritici (the causal agent of wheat leaf rust) and incubated in a saturated atmosphere at 20° C. for 24 h, and then moved to a growth chamber at 20° C. for 7 days, after which time disease ratings were visually made.

### Test C

The test suspension was sprayed to the point of run-off on wheat seedlings. The following day the seedlings were inoculated with a spore suspension of Septoria tritici (the causal agent of wheat leaf blotch) and incubated in a saturated atmosphere at 20° C. for 48 h, and moved to a growth chamber at 20° C. for 19 additional days, after which time disease ratings were visually made.

### Test D

The test suspension was sprayed to the point of run-off on wheat seedlings. The following day the seedlings were inoculated with a spore suspension of Septoria nodorum (the causal agent of wheat glume blotch) and incubated in a saturated atmosphere at 20° C. for 48 h, and then moved to a growth chamber at 20° C. for 7 days, after which time disease ratings were visually made.

### Test E

The test suspension was sprayed to the point of run-off on tomato seedlings. The following day the seedlings were inoculated with a spore suspension of Alternaria solani (the causal agent of tomato early blight) and incubated in a saturated atmosphere at 27° C. for 48 h, and then moved to a growth chamber at 20° C. for 5 days, after which time disease ratings were visually made.

### Test F

The test suspension was sprayed to the point of run-off on tomato seedlings. The following day the seedlings were inoculated with a spore suspension of Botrytis cinerea (the causal agent of tomato botrytis) and incubated in saturated atmosphere at 20° C. for 48 h, and then moved to a growth chamber at 24° C. for 3 additional days, after which time disease ratings were visually made.

### Test G

The test suspension was sprayed to the point of run-off on creeping bent grass seedlings. The following day the seedlings were inoculated with a spore suspension of Rhizoctonia oryzae (the causal agent of turf brown patch) and incubated in a saturated atmosphere at 27° C. for 48 h, and then moved to a growth chamber at 27° C. for 3 days, after which time disease ratings were visually made.

### Test H

Grape seedlings were inoculated with a spore suspension of Plasmopara viticola (the causal agent of grape downy mildew) and incubated in a saturated atmosphere at 20° C. for 24 h. After a short drying period, the test suspension was sprayed to the point of run-off on the grape seedlings, and then moved to a growth chamber at 20° C. for 6 days, after which time the test units were placed back into a saturated atmosphere at 20° C. for 24 h. Upon removal, disease ratings were visually made.

Results for Tests A-H are given in Table A. In the table, a rating of 100 indicates 100% disease control and a rating of 0 indicates no disease control (relative to the controls). A dash (-) indicates no test results. All results are for compounds tested at 200 ppm except where the compound number is followed by “*” which indicates the compound was tested at 40 ppm.

TABLE A

| Compound No. | Test A | Test B | Test C | Test D | Test E | Test F | Test G | Test H |
|---|---|---|---|---|---|---|---|---|
| 1 | 96 | 28 | — | 0 | 0 | 99 | — | 0 |
| 2 | 95 | 0 | — | 0 | 0 | 99 | — | 0 |
| 4 | 99 | 92 | 99 | 0 | 99 | 99 | 0 | 0 |
| 5 | 98 | 92 | 95 | 0 | 90 | 99 | 0 | 0 |
| 6 | 0 | 0 | 1 | 0 | 0 | 89 | 0 | 0 |
| 7 | 97 | 74 | 52 | 0 | 0 | 98 | 97 | 0 |
| 8 | 98 | 0 | 0 | 0 | 0 | 0 | 0 | 17 |
| 9 | 94 | 0 | 86 | 0 | 99 | 37 | 0 | 0 |
| 10 | 96 | 0 | 33 | 0 | 8 | 80 | 0 | 0 |
| 11 | 98 | 0 | 30 | 0 | 0 | 0 | — | 0 |
| 12 | 95 | 93 | 92 | 0 | 90 | 99 | — | 0 |
| 13 | 96 | 99 | 100 | 0 | 100 | 99 | — | 0 |
| 14 | 97 | 98 | 99 | 0 | 99 | 99 | — | 0 |
| 15 | 82 | 0 | 7 | 0 | 0 | 96 | — | 0 |
| 16 | 99 | 100 | 100 | 73 | 99 | 99 | 99 | 0 |
| 17 | 96 | 98 | 100 | 0 | 46 | 99 | 60 | 0 |
| 18 | 98 | 99 | 100 | 0 | — | 97 | 47 | 0 |
| 19 | 99 | 99 | 100 | 0 | 99 | 99 | 99 | 0 |
| 20 | 98 | 90 | 100 | 0 | 0 | 99 | 0 | 53 |
| 21 | 100 | 99 | 100 | 0 | 97 | 98 | — | 40 |
| 22 | 78 | 55 | 95 | 0 | 0 | 99 | 0 | 0 |
| 23 | 62 | 80 | 83 | 0 | 0 | 99 | 0 | 0 |
| 24* | 91 | 95 | 97 | 0 | 76 | 99 | 0 | 0 |
| 25* | 0 | 0 | 0 | 0 | 0 | 84 | — | 0 |
| 26 | 94 | 68 | 86 | 0 | 0 | 98 | — | 0 |
| 27 | 98 | 80 | 100 | 0 | 92 | 99 | — | 0 |
| 28 | 96 | 28 | 100 | 0 | 93 | 99 | — | 0 |
| 29* | 91 | 0 | 98 | 0 | 91 | 80 | — | — |
| 30 | 98 | 80 | 100 | 0 | 0 | 99 | — | 0 |
| 31 | 0 | 0 | 80 | 0 | 0 | 41 | 0 | 0 |
| 32 | 98 | 96 | 99 | 0 | 59 | 99 | 99 | 0 |
| 33 | 79 | — | — | — | 100 | 95 | — | 0 |
| 34 | 96 | 96 | 100 | 77 | 96 | 99 | 63 | 0 |
| 35 | 99 | 100 | 99 | 99 | 99 | 99 | 99 | 0 |
| 36 | 65 | 0 | 93 | 0 | 30 | 94 | 14 | 0 |
| 38 | 95 | 80 | 100 | 0 | 87 | 98 | 56 | 0 |
| 39 | 98 | 100 | 100 | 100 | 100 | 94 | 35 | 0 |
| 40 | 94 | 80 | 87 | 0 | 97 | 90 | 49 | 0 |
| 41 | 100 | 98 | 99 | 0 | 99 | 98 | 99 | 0 |
| 42 | 100 | 100 | 100 | 98 | — | 97 | 86 | 0 |
| 43 | 100 | 100 | 100 | 78 | 100 | 100 | 100 | — |
| 44 | 99 | 100 | 100 | 99 | 100 | 99 | 86 | — |
| 45 | 99 | 100 | 100 | 0 | 94 | 100 | 99 | — |
| 46 | 81 | 95 | — | 0 | 31 | 98 | 0 | — |
| 47 | 91 | 97 | — | 0 | 67 | 96 | 99 | — |
| 48* | 98 | 100 | — | 0 | 99 | 98 | 100 | — |
| 49 | 0 | 0 | — | 0 | 16 | 0 | 0 | — |
| 50 | 92 | 99 | — | 60 | 100 | 97 | 98 | — |
| 51 | 76 | 0 | — | 0 | 0 | 0 | | 0 |
| 52 | 25 | 0 | — | 0 | 0 | 0 | 0 | 0 |
| 54 | 0 | 54 | 47 | 0 | 0 | 0 | 0 | 0 |
| 55 | 0 | 27 | 72 | 0 | 0 | 0 | 0 | 0 |
| 58 | 77 | 0 | 10 | 0 | 31 | 0 | — | 0 |
| 59 | 79 | 100 | 97 | 92 | 100 | 100 | — | — |
| 60 | 59 | 59 | 68 | 0 | 0 | 99 | — | — |
| 61 | 86 | 99 | 84 | 0 | 83 | 99 | — | — |
| 62 | 80 | 100 | 94 | 73 | 100 | 95 | — | — |
| 63 | 78 | 96 | 79 | 0 | 74 | 97 | — | — |
| 64 | 85 | 99 | 95 | 90 | 100 | 99 | — | — |
| 65 | 80 | 98 | 96 | 0 | 100 | 99 | — | — |
| 66 | 99 | 98 | 98 | 0 | 99 | 95 | — | — |
| 67* | 100 | 100 | 95 | 0 | 99 | 99 | — | — |
| 68 | 100 | 99 | 88 | 0 | 99 | 99 | — | — |
| 69 | 99 | 100 | 94 | 86 | 100 | 91 | — | — |
| 70 | 94 | 95 | 93 | 0 | 79 | 97 | — | — |
| 71 | 98 | 100 | 95 | 81 | 100 | 99 | — | — |
| 72 | 100 | 100 | 95 | 100 | 100 | 99 | — | — |
| 73* | 100 | 100 | 94 | 0 | 91 | 99 | — | — |
| 74 | 100 | 100 | 98 | 100 | 100 | 98 | — | — |
| 75* | 99 | 100 | 96 | 99 | 100 | 99 | — | — |
| 76* | 0 | 28 | 0 | 0 | 0 | 0 | — | — |
| 77* | 100 | 99 | 92 | 0 | 99 | 84 | — | — |
| 78* | 100 | 99 | 92 | 0 | 96 | — | — | — |
| 79* | 97 | 100 | 93 | 95 | 100 | 91 | — | — |
| 80 | 95 | 97 | 57 | 0 | 64 | 0 | — | — |
| 81 | 78 | 96 | 93 | 0 | 98 | 46 | — | — |
| 82 | 97 | 98 | 95 | 69 | 98 | 98 | — | — |
| 83 | 95 | 99 | 95 | 0 | 99 | 54 | — | — |
| 84* | 96 | 98 | 96 | 0 | 0 | 49 | — | — |
| 85 | 43 | 61 | 43 | 0 | 0 | 96 | — | — |
| 86* | 96 | 98 | 93 | 0 | 31 | 92 | — | — |
| 87* | 98 | 96 | 95 | 0 | 0 | 72 | — | — |
| 88* | 58 | 53 | 77 | 0 | 0 | 99 | — | — |
| 89* | 100 | 100 | 97 | 99 | 99 | 100 | — | — |
| 90* | 0 | 74 | 90 | 0 | 98 | 71 | — | — |
| 92* | 76 | 99 | 96 | 82 | 100 | 94 | — | — |
| 93* | 96 | 91 | 93 | 0 | 21 | 98 | — | — |
| 94 | 21 | 54 | 91 | 0 | 0 | 98 | — | — |
| 95 | 93 | 99 | 96 | 0 | 73 | 87 | — | — |
| 96 | 98 | 96 | 94 | 0 | 99 | 98 | — | — |
| 97 | 100 | 100 | 95 | 100 | 100 | 99 | — | — |
| 98 | 86 | 92 | 92 | 0 | 9 | 93 | — | — |
| 99 | 100 | 100 | 94 | 100 | 100 | 98 | — | — |
| 100 | 76 | 89 | 76 | 0 | 0 | 100 | — | — |
| 101 | 82 | 89 | 96 | 0 | 0 | 96 | — | — |
| 102 | 98 | 92 | 97 | 0 | 91 | 98 | — | — |
| 103 | 100 | 100 | 95 | 98 | 100 | 99 | — | — |
| 104 | 100 | 100 | 98 | 99 | 100 | 99 | — | — |
| 105 | 100 | 99 | 93 | 0 | 0 | 98 | — | — |
| 106 | 96 | 100 | 98 | 0 | 99 | 98 | — | — |
| 107 | 100 | 100 | 98 | 98 | 100 | 99 | — | — |
| 108 | 64 | 85 | 71 | 0 | 0 | 87 | — | — |
| 109 | 98 | 97 | 97 | 0 | 0 | 92 | — | — |
| 110 | 100 | 100 | 96 | 87 | 100 | 99 | — | — |
| 111 | 93 | 97 | 91 | 0 | 0 | 99 | — | — |
| 112* | 96 | 80 | 93 | 0 | 9 | 96 | — | — |
| 113* | 0 | 89 | 76 | 0 | 9 | 79 | — | — |
| 114* | 89 | 99 | 99 | 69 | 94 | 99 | — | — |
| 115* | 93 | 96 | 98 | 0 | 99 | 98 | — | — |
| 116* | 94 | 89 | 95 | 0 | 99 | 99 | — | — |
| 117* | 93 | 100 | 99 | 82 | 99 | 99 | — | — |
| 118* | 100 | 100 | 96 | 0 | 99 | 100 | — | — |
| 119* | 99 | 100 | 98 | 98 | 100 | 100 | — | — |
| 120* | 0 | 91 | 87 | 0 | 47 | 79 | — | — |
| 121* | 99 | 100 | 99 | 94 | 99 | 100 | — | — |
| 122* | 98 | 99 | 99 | 0 | 0 | 100 | — | — |
| 123* | 0 | 0 | 53 | 0 | 0 | 69 | — | — |
| 124* | 100 | 100 | 99 | 100 | 100 | 100 | — | — |
| 125* | 96 | 99 | 96 | 90 | 99 | 100 | — | — |
| 126* | 90 | 100 | 99 | 100 | 97 | 100 | — | — |
| 127* | 90 | 97 | 99 | 0 | 0 | 99 | — | — |
| 128* | 81 | 99 | 100 | 90 | 94 | 99 | — | — |
| 129* | 99 | 100 | 99 | 60 | 66 | 99 | — | — |
| 130 | 100 | 93 | — | 0 | 99 | 100 | — | — |
| 131* | 0 | 9 | 0 | 0 | 0 | 80 | — | — |
| 132* | 90 | 99 | 99 | 97 | 93 | 99 | — | — |
| 133* | 98 | 100 | 100 | 95 | 99 | 100 | — | — |
| 134* | 99 | 100 | 99 | 92 | 96 | 100 | — | — |
| 135* | 98 | 100 | 99 | 96 | 88 | 100 | — | — |
| 136* | 0 | 74 | 40 | 0 | 0 | 48 | — | — |
| 137* | 100 | 100 | 100 | 97 | 99 | 100 | — | — |
| 143* | 99 | 97 | 100 | 0 | 97 | 28 | — | — |
| 144* | 0 | 0 | 0 | 0 | 0 | 45 | — | — |

What is claimed is:

1. A compound selected from Formula 1, N-oxides and salts thereof, 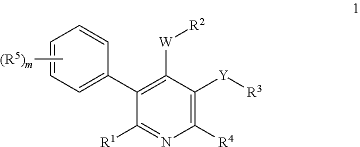
wherein
R¹ is halogen, cyano, hydroxy, amino, C₁-C₄ alkyl, C₂-C₄ alkenyl, C₂-C₄ alkynyl, C₁-C₄ haloalkyl, C₂-C₄ haloalkenyl, C₂-C₄ haloalkynyl, cyclopropyl, halocyclopropyl, C₂-C₄ alkoxyalkyl, C₂-C₄ alkylthioalkyl, C₂-C₄ alkylsulfinylalkyl, C₂-C₄ alkylsulfonylalkyl, C₂-C₄ alkylcarbonyl, C₂-C₄ alkoxycarbonyl, C₁-C₃ hydroxyalkyl, C₁-C₃ alkoxy, C₁-C₃ haloalkoxy, C₁-C₃ alkylthio, C₁-C₃ haloalkylthio, C₁-C₃ alkylsulfinyl, C₁-C₃ haloalkylsulfinyl, C₁-C₃ alkylsulfonyl, C₁-C₃ haloalkylsulfonyl, C₁-C₃ alkylamino or C₂-C₄ dialkylamino;
each W and Y is independently CH₂, O, C(═O), S(═O)ₙ, NR⁸ or a direct bond;
R² is a phenyl ring optionally substituted with up to 5 substituents independently selected from R⁶; or a 3-, 4-, 5- or 6-membered heterocyclic ring containing ring members selected from carbon atoms and up to 4 heteroatoms selected from up to 2 oxygen, up to 2 sulfur and up to 3 nitrogen atoms, wherein up to 3 carbon atom ring members are independently selected from C(═O) and C(═S), and the sulfur atom ring members are independently selected from S(═O)ₚ(═NR⁹)_{q}, the heterocyclic ring optionally substituted with up to 5 substituents independently selected from R⁶ on carbon atom ring members and R^{6a} on nitrogen atom ring members;
R³ is a phenyl ring optionally substituted with up to 5 substituents independently selected from R⁷; or a 3-, 4-, 5- or 6-membered heterocyclic ring containing ring members selected from carbon atoms and up to 4 heteroatoms selected from up to 2 oxygen, up to 2 sulfur and up to 3 nitrogen atoms, wherein up to 3 carbon atom ring members are independently selected from C(═O) and C(═S), and the sulfur atom ring members are independently selected from S(═O)ₚ(═NR⁹)_{q}, the heterocyclic ring optionally substituted with up to 5 substituents independently selected from R⁷ on carbon atom ring members and R^{7a} on nitrogen atom ring members; or
when Y is a direct bond, then R³ is also selected from halogen, cyano, hydroxy, amino, nitro, —CHO, C₁-C₆ alkyl, C₂-C₆ alkenyl, C₂-C₆ alkynyl, C₁-C₆ haloalkyl, C₂-C₆ haloalkenyl, C₂-C₆ haloalkynyl, C₃-C₆ cycloalkyl, C₃-C₆ halocycloalkyl, C₄-C₈ alkylcycloalkyl, C₄-C₈ cycloalkylalkyl, C₆-C₁₂ cycloalkylcycloalkyl, C₄-C₈ halocycloalkylalkyl, C₅-C₈ alkylcycloalkylalkyl, C₃-C₆ cycloalkenyl, C₂-C₆ alkoxyalkyl, C₂-C₆ alkylthioalkyl, C₂-C₆ alkylsulfinylalkyl, C₂-C₆ alkylsulfonylalkyl, C₂-C₆ alkylaminoalkyl, C₃-C₆ dialkylaminoalkyl, C₂-C₆ alkylcarbonyl, C₂-C₆ haloalkylcarbonyl, C₄-C₆ cycloalkylcarbonyl, C₂-C₆ alkoxycarbonyl, C₂-C₆ alkylaminocarbonyl, C₃-C_{g} dialkylaminocarbonyl, C₂-C₆ cyanoalkyl, C₁-C₆ hydroxyalkyl, C₂-C₆ hydroxyhaloalkyl, C₂-C₆ hydroxyalkylcarbonyl, C₂-C₆ hydroxycarbonylalkyl, C₁-C₆ alkoxy, C₁-C₆ haloalkoxy, C₃-C₆ cycloalkoxy, C₃-C₆ halocycloalkoxy, C₂-C₆ alkoxyalkoxy, C₃-C₆ alkoxycarbonylalkyl, C₁-C₆ alkylthio, C₁-C₆ haloalkylthio, C₁-C₆ alkylsulfinyl, C₁-C₆ haloalkylsulfinyl, C₁-C₆ alkylsulfonyl, C₁-C₆ haloalkylsulfonyl, C₃-C₉ trialkylsilyl, C₁-C₆ alkylamino, C₂-C₆ dialkylamino, C₂-C₆ haloalkylamino, C₂-C₆ halodialkylamino, C₃-C₆ cycloalkylamino, C₂-C₆ alkylcarbonylamino, C₂-C₆ haloalkylcarbonylamino, C₁-C₆ alkylsulfonylamino and C₁-C₆ haloalkylsulfonylamino;
R⁴ is H, halogen, cyano, hydroxy, C₁-C₂ alkyl, C₁-C₂ haloalkyl, C₂ alkenyl, C₂ haloalkenyl or C₂ alkynyl;
each R⁵, R⁶ and R⁷ is independently halogen, cyano, hydroxy, amino, nitro, —CHO, C₁-C₆ alkyl, C₂-C₆ alkenyl, C₂-C₆ alkynyl, C₁-C₆ haloalkyl, C₂-C₆ alkylcarbonyl, C₂-C₆ haloalkylcarbonyl, C₂-C₆ alkoxycarbonyl, C₂-C₆ alkylaminocarbonyl, C₃-C₆ dialkylaminocarbonyl, C₂-C₆ alkylaminoalkoxy, C₂-C₆ haloalkenyl, C₂-C₆ haloalkynyl, C₃-C₆ cycloalkyl, C₃-C₆ halocycloalkyl, C₄-C₈ alkylcycloalkyl, C₄-C₈ cycloalkylalkyl, C₅-C₈ alkylcycloalkylalkyl, C₂-C₆ alkoxyalkyl, C₂-C₆ cyanoalkyl, C₁-C₆ hydroxyalkyl, C₁-C₆ alkoxy, C₁-C₆ haloalkoxy, C₃-C₆ cycloalkoxy, C₃-C₆ halocycloalkoxy, C₂-C₆ alkylcarbonyloxy, C₁-C₆ alkylthio, C₁-C₆ haloalkylthio, C₁-C₆ alkylsulfinyl, C₁-C₆ haloalkylsulfinyl, C₁-C₆ alkylsulfonyl, C₁-C₆ haloalkylsulfonyl, C₃-C₉ trialkylsilyl, C₂-C₆ alkylcarbonylthio, C₁-C₆ alkylamino or C₂-C₆ dialkylamino;
each R^{6a} and R^{7a} is independently cyano, C₁-C₆ alkyl, C₂-C₆ alkenyl, C₂-C₆ alkynyl, C₁-C₆ haloalkyl, C₂-C₆ alkylcarbonyl, C₂-C₆ haloalkylcarbonyl, C₂-C₆ alkoxycarbonyl, C₂-C₆ alkylaminocarbonyl, C₃-C₆ dialkylaminocarbonyl, C₂-C₆ haloalkenyl, C₂-C₆ haloalkynyl, C₃-C₆ cycloalkyl, C₃-C₆ halocycloalkyl, C₄-C₈ alkylcycloalkyl, C₄-C₈ cycloalkylalkyl, C₅-C₈ alkylcycloalkylalkyl, C₂-C₆ alkoxyalkyl, C₁-C₆ alkoxy, C₁-C₆ haloalkoxy, C₃-C₆ cycloalkoxy, C₃-C₆ halocycloalkoxy, C₁-C₆ alkylthio, C₁-C₆ haloalkylthio, C₁-C₆ alkylsulfonyl, C₁-C₆ haloalkylsulfonyl or C₃-C₉ trialkylsilyl; or
one pair of R⁵ substituents attached to adjacent ring atoms, one pair of substituents selected from R⁶ and R^{6a} substituents attached to adjacent ring atoms, and one pair of substituents selected from R⁷ and R^{7a} substituents attached to adjacent ring atoms may each be independently taken together with the atoms to which they are attached to form a 5-, 6- or 7-membered fused ring, each fused ring containing ring members selected from carbon and up to 4 heteroatoms selected from up to 2 oxygen, up to 2 sulfur and up to 3 nitrogen, and optionally substituted with up to 3 substituents independently selected from the group consisting of C₁-C₂ alkyl, halogen, cyano, nitro and C₁-C₂ alkoxy on carbon ring members and from the group consisting of C₁-C₂ alkyl, cyano and C₁-C₂ alkoxy on nitrogen ring members; or
one pair of R⁶ substituents attached to the same ring atom and one pair of R⁷ substituents attached to the same ring atom may each be independently taken together with the atom to which they are attached to form a 5-, 6- or 7-membered spirocyclic ring, each spirocyclic ring containing ring members selected from carbon, up to 4 heteroatoms selected from up to 2 oxygen, up to 2 sulfur and up to 3 nitrogen, and optionally substituted with up to 3 substituents independently selected from the group consisting of C₁-C₂ alkyl, halogen, cyano, nitro and C₁-C₂ alkoxy on carbon ring members and from the group consisting of C₁-C₂ alkyl, cyano and C₁-C₂ alkoxy on nitrogen ring members;
each R⁸ and R⁹ is independently H or C₁-C₃ alkyl;
m is an integer selected from 0, 1, 2, 3, 4 and 5;
each n is independently an integer selected from 0, 1 and 2; and
p and q are independently 0, 1 or 2 in each instance of S(═O)ₚ(═NR⁹)_{q}, provided that the sum of p and q is 0, 1 or 2;
provided that when Y is a direct bond and R³ is a phenyl ring substituted with two alkoxy substituents attached at the meta positions, then R⁴ is H.

2. A compound of claim 1 wherein
R¹ is halogen, cyano, C₁-C₄ alkyl, C₂-C₄ alkenyl, C₁-C₄ haloalkyl, C₁-C₃ alkoxy, C₁-C₃ haloalkoxy or C₁-C₃ alkylthio;
R² is a phenyl ring optionally substituted with up to 5 substituents independently selected from R⁶; or a 5- or 6-membered heterocyclic ring containing ring members selected from carbon atoms and up to 4 heteroatoms selected from up to 2 oxygen, up to 2 sulfur and up to 3 nitrogen atoms, wherein up to 3 carbon atom ring members are independently selected from C(═O) and C(═S), and the sulfur atom ring members are independently selected from S(═O)ₚ(═NR⁹)_{q}, the heterocyclic ring optionally substituted with up to 5 substituents selected from R⁶ on carbon atom ring members and R^{6a} on nitrogen atom ring members;
R³ is a phenyl ring optionally substituted with up to 5 substituents independently selected from R⁷; or a 5- or 6-membered heterocyclic ring containing ring members selected from carbon atoms and up to 4 heteroatoms selected from up to 2 oxygen, up to 2 sulfur and up to 3 nitrogen atoms, wherein up to 3 carbon atom ring members are independently selected from C(═O) and C(═S), and the sulfur atom ring members are independently selected from S(═O)ₚ(═NR⁹)_{q}, the heterocyclic ring optionally substituted with up to 5 substituents selected from R⁷ on carbon atom ring members and R^{7a} on nitrogen atom ring members; or
when Y is a direct bond, then R³ is also selected from halogen, cyano, C₁-C₆ alkyl, C₂-C₆ alkenyl, C₁-C₆ haloalkyl, C₂-C₆ haloalkenyl, C₃-C₆ cycloalkyl, C₂-C₆ alkylcarbonyl, C₂-C₆ alkoxycarbonyl, C₂-C₆ cyanoalkyl and C₁-C₆ hydroxyalkyl;
each R⁵, R⁶ and R⁷ is independently halogen, cyano, C₁-C₆ alkyl, C₂-C₆ alkenyl, C₁-C₆ haloalkyl, C₁-C₆ alkoxy, C₁-C₆ haloalkoxy, C₁-C₆ alkylthio or C₁-C₆ haloalkylthio; and
each R^{6a} and R^{7a} is independently cyano, C₁-C₆ alkyl, C₂-C₆ alkenyl, C₁-C₆ haloalkyl, C₁-C₆ alkoxy, C₁-C₆ haloalkoxy, C₁-C₆ alkylthio or C₁-C₆ haloalkylthio.

3. The compound of claim 2 wherein
R¹ is halogen, cyano or C₁-C₄ alkyl;
each W and Y is independently CH₂, O, S or a direct bond;
R² is a phenyl ring optionally substituted with up to 3 substituents independently selected from R⁶; or a 5- or 6-membered heterocyclic ring containing ring members selected from carbon atoms and up to 4 heteroatoms selected from up to 2 oxygen, up to 2 sulfur and up to 3 nitrogen atoms, wherein up to 3 carbon atom ring members are independently selected from C(═O) and C(═S), and the sulfur atom ring members are independently selected from S(═O)ₚ(═NR⁹)_{q}, the heterocyclic ring optionally substituted with up to 3 substituents selected from R⁶ on carbon atom ring members and R^{6a} on nitrogen atom ring members; and
R³ is a phenyl ring optionally substituted with up to 3 substituents independently selected from R⁷; or a 5- or 6-membered heterocyclic ring containing ring members selected from carbon atoms and up to 4 heteroatoms selected from up to 2 oxygen, up to 2 sulfur and up to 3 nitrogen atoms, wherein up to 3 carbon atom ring members are independently selected from C(═O) and C(═S), and the sulfur atom ring members are independently selected from S(═O)ₚ(═NR⁹)_{q}, the heterocyclic ring optionally substituted with up to 3 substituents selected from R⁷ on carbon atom ring members and R^{7a} on nitrogen atom ring members; or
when Y is a direct bond, then R³ is also selected from C₁-C₆ alkyl, C₂-C₆ alkenyl, C₁-C₆ haloalkyl, C₃-C₆ cycloalkyl, C₂-C₆ alkylcarbonyl, C₂-C₆ alkoxycarbonyl, C₂-C₆ cyanoalkyl and C₁-C₆ hydroxyalkyl.

4. The compound of claim 3 wherein
R¹ is halogen or C₁-C₂ alkyl;
W is a direct bond;
Y is a direct bond;
R² is a phenyl ring optionally substituted with up to 3 substituents independently selected from R⁶;
R³ is a phenyl ring optionally substituted with up to 3 substituents independently selected from R⁷; or
R³ is C₁-C₆ alkyl, C₂-C₆ alkenyl, C₁-C₆ haloalkyl, C₂-C₆ cyanoalkyl or C₁-C₆ hydroxyalkyl;
R⁴ is H, cyano or C₁-C₂ alkyl;
each R⁵, R⁶ and R⁷ is independently halogen, C₁-C₆ alkyl, C₂-C₆ alkenyl, C₁-C₆ haloalkyl or C₁-C₆ alkoxy; and
each R^{6a} and R^{7a} is independently C₁-C₆ alkyl, C₂-C₆ alkenyl, C₁-C₆ haloalkyl or C₁-C₆ alkoxy.

5. The compound of claim 4 wherein
R¹ is methyl;
R² is a phenyl ring optionally substituted with up to 2 substituents independently selected from R⁶;
R³ is a phenyl ring optionally substituted with up to 1 substituent selected from R⁷; or
R³ is C₁-C₄ alkyl, C₁-C₃ haloalkyl, C₂-C₄ cyanoalkyl and C₁-C₄ hydroxyalkyl;
R⁴ is H;
each R⁵, R⁶ and R⁷ is independently halogen, C₁-C₆ alkyl or C₁-C₆ alkoxy;
each R^{6a} and R^{7a} is independently C₁-C₆ alkyl; and
m is an integer selected from 0, 1, 2 and 3.

6. The compound of claim 5 wherein
each R⁵ is independently halogen or methoxy; and
each R⁶ is independently chlorine or methoxy.

7. A compound of claim 1 selected from the group consisting of:
4-(3,5-dimethoxyphenyl)-6-methyl-5-(2,4,6-trifluorophenyl)-3-pyridineacetonitrile,
4-(3,5-dimethoxyphenyl)-5-(2-fluorophenyl)-2-methyl-3-(2,4,6-trifluorophenyl)pyridine,
5-(2,6-difluoro-4-methoxyphenyl)-4-(3,5-dimethoxyphenyl)-α,α,6-trimethyl-3-pyridinemethanol,
5-(chloromethyl)-4-(3,5-dimethoxyphenyl)-2-methyl-3-(2,4,6-trifluorophenyl)pyridine,
4-(3,5-dimethoxyphenyl)-2-methyl-5-phenyl-3-(2,4,6-trifluorophenyl)pyridine,
4-(2-chloro-3,5-dimethoxyphenyl)-6-methyl-5-(2,4,6-trifluorophenyl)-3-pyridineacetonitrile,
4-(2-chloro-3,5-dimethoxyphenyl)-5-(2-fluorophenyl)-2-methyl-3-(2,4,6-trifluorophenyl)pyridine, and
4-(3,5-dimethoxyphenyl)-5-ethyl-2-methyl-3-(2,4,6-trifluorophenyl)pyridine.

8. A fungicidal composition comprising (a) a compound of claim 1; and (b) at least one other fungicide.

9. A fungicidal composition comprising (a) a compound of claim 1; and (b) at least one additional component selected from the group consisting of surfactants, solid diluents and liquid diluents.

10. A method for controlling plant diseases caused by fungal plant pathogens comprising applying to the plant or portion thereof, or to the plant seed, a fungicidally effective amount of a compound of claim 1.
